SEQUENCE LISTING (14 sequences)

SEQ ID NO: 1 - DNA, 1632 bp, synthetic construct
  source 1..1632: mol_type = other DNA
atgtgccctcagaagctgaccatcagttggttcgccatcgtgctgctggtgtccccactgatggctatgtgggaactcgagaaggacgtgtacgtggtggaagtggactggacccctgatgctcctggcgagacagtgaacctgacctgcgacacacctgaagaggacgacatcacctggaccagcgatcagagacacggcgtgatcggctctggcaagaccctgacaattaccgtgaaagagttcctggacgccggccagtacacctgtcacaaaggcggagagacactgagccactctcatctgctgctgcacaagaaagagaacggcatctggtccaccgagatcctgaagaacttcaagaacaagaccttcctgaagtgcgaggcccctaactacagcggcagattcacctgtagctggctggtgcagagaaacatggacctgaagttcaacatcaagtcctccagcagcagccccgacagcagagctgtgacatgtggcatggctagcctgagcgccgagaaagtgacactggaccagagagactacgagaagtacagcgtgtcctgccaagaggacgtgacctgtcctaccgccgaggaaacactgcctatcgagctggccctggaagccagacagcagaacaaatacgagaactactctaccagcttcttcatccgggacatcatcaagcccgatcctccaaagaacctgcagatgaagcctctgaagaacagccaggtcgaggtgtcctgggagtaccctgacagctggtctacccctcacagctacttcagcctgaaattcttcgtgcggatccagcgcaagaaagaaaagatgaaggaaaccgaggaaggctgcaaccagaaaggcgctttcctggtggaaaagaccagcaccgaggtgcagtgcaaaggcggcaatgtctgtgtgcaggcccaggaccggtactacaacagcagctgtagcaagtgggcctgcgtgccatgcagagtcagatctggtggcggaggatctggcggaggtggaagcggcggaggcggatctagagtgattcctgtgtctggccctgccagatgcctgagccagtctagaaacctgctgaaaaccaccgacgacatggtcaagaccgccagagagaagctgaagcactactcctgcacagccgaggacatcgatcacgaggatatcaccagggaccagacaagcaccctgaaaacctgcctgcctctggaactgcataagaacgagagctgcctggccaccagagaaaccagctctaccacaagaggcagctgtctgcctcctcagaaaaccagcctgatgatgaccctgtgcctgggcagcatctacgaggatctgaagatgtaccagaccgagttccaggccatcaacgccgctctgcagaaccacaaccaccagcagatcatcctggacaagggcatgctggtggctatcgacgagctgatgcagagcctgaaccataacggcgagacactgcggcagaagcctccagttggagaggccgatccttacagagtgaagatgaagctgtgcatcctgctgcacgccttcagcaccagagtggtcaccatcaacagagtgatgggctacctgagcagcgcctga

SEQ ID NO: 2 - protein, 543 aa, synthetic construct
MCPQKLTISWFAIVLLVSPLMAMWELEKDVYVVEVDWTPDAPGETVNLTCDTPEEDDITWTSDQRHGVIGSGKTLTITVKEFLDAGQYTCHKGGETLSHSHLLLHKKENGIWSTEILKNFKNKTFLKCEAPNYSGRFTCSWLVQRNMDLKFNIKSSSSSPDSRAVTCGMASLSAEKVTLDQRDYEKYSVSCQEDVTCPTAEETLPIELALEARQQNKYENYSTSFFIRDIIKPDPPKNLQMKPLKNSQVEVSWEYPDSWSTPHSYFSLKFFVRIQRKKEKMKETEEGCNQKGAFLVEKTSTEVQCKGGNVCVQAQDRYYNSSCSKWACVPCRVRSGGGGSGGGGSGGGGSRVIPVSGPARCLSQSRNLLKTTDDMVKTAREKLKHYSCTAEDIDHEDITRDQTSTLKTCLPLELHKNESCLATRETSSTTRGSCLPPQKTSLMMTLCLGSIYEDLKMYQTEFQAINAALQNHNHQQIILDKGMLVAIDELMQSLNHNGETLRQKPPVGEADPYRVKMKLCILLHAFSTRVVTINRVMGYLSSA

SEQ ID NO: 3 - DNA, 930 bp, Mus musculus
  source 1..930: mol_type = genomic DNA
atggaccagcacacactggacgtggaagataccgccgacgccagacaccctgccggcacaagctgtccatctgatgccgccctgctgagagacacaggcctgctggctgatgctgctctgctgtctgacaccgtgcggcctacaaacgccgctctgcctacagatgccgcctaccctgctgtgaacgtgcgggatagagaggccgcttggcctcccgccctgaacttctgcagcagacaccccaagctgtacggcctggtggctctggtgctcctgctgctgattgccgcctgcgtgcccatcttcaccagaaccgagcctagacctgccctgaccatcaccaccagccctaacctgggcaccagagagaacaacgccgaccaagtgacccccgtgtcccacatcggctgccctaacacaacccagcagggcagccctgtgttcgccaagctgctggccaagaaccaggccagcctgtgcaacaccaccctgaactggcacagccaggacggcgctggcagcagctatctgagccagggcctgagatacgaagaggacaagaaagaactggtggtggacagccctggcctgtactacgtgttcctggaactgaagctgagccccaccttcaccaacaccggccacaaggtgcagggctgggtgtcactggtgctgcaggctaagcctcaggtggacgacttcgacaacctggccctgacagtggaactgttcccctgcagcatggaaaacaagctggtggatagaagctggtcccagctgctgctgctgaaggccggccatagactgagcgtgggcctgagggcttatctgcacggcgcccaggacgcctacagagactgggagctgagctaccccaacacaaccagcttcggcctgttcctcgtgaagcccgacaacccctgggagtga

SEQ ID NO: 4 - protein, 309 aa, Mus musculus
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 5 - DNA, 1980 bp, Mus musculus
  source 1..1980: mol_type = genomic DNA
atggaaaccgacacactgctgctgtgggtgctgcttctttgggtgcccggatctacaggcgacgtggcacttggaaacagccctcaccaggtgttcaacctgagctgggaagtgacaaacggcgaccgcgaaacagtgtgggccatcacaggcaatcaccctctgtggacctggtggcctgacctgacacctgacctgtgtatgctggctctgcacggcccatcttactggggcctcgagtacagagcccctttctctcctccacctggacctccatgttgtagcggcagcagcgacagcacaagcggctgttctagagactgcgaggaacccctgaccagctacacccctagatgtaacaccgcctggaacagactgaagctgagcaaagtgacacacgcccacaacgagggcttctacgtgtgtcctggaccacacagacccagatgggccagatcttgtggcggccctgagagcttctactgtgctagctggggctgcgagacaaccggcagagcttcttggaagcctagcagcagctgggactacatcaccgtgtccaacaacctgacctccgaccaggctacccctgtgtgcaagggcaacaagtggtgcaacagcctgaccatcagattcaccagcttcggcaagcaggccacctcttgggtcacaggacattggtggggcctgagactgtatgtgtccggccatgatcctggcctgatcttcggcatcaggctgaagatcacagacagcggccccagagtgcctatcggccctaatcctgtgctgagcgacagaaggcctcctagcagacccaggcctacaagatctccacctccaagcaacagcacccctaccgagacacctctgacactgcctgaacctccaccagccggcgtggaaaacagactgctgaatctggtcaagggcgcctaccaggctctgaacctgaccagccctgataagacacaagagtgctggctgtgcctggtgtctggccctccttactatgaaggcgtggccgtgctgggcacctacagcaatcatacaagcgcccctgccaactgcagcgtggcctctcagcataagctgaccctgtctgaagtgaccggccagggcctgtgtattggcgctgtgcctaagacacaccaggtgctgtgcaacacaacccagaaaaccagcgacggcagctactacctggctgctcctacaggcacaacctgggcctgtagcacaggactgaccccttgtatcagcaccaccatcctgaatctgaccaccgactactgcgtgctggtggaactgtggcctagagtgacctaccactctcctagctacgtgtaccaccagttcgagagaagggccaagtacaagcgcgagcccgtgtctcttacactggccttgcttctcggcggcctgacaatgggaggaatcgctgctggtgtcggcaccggaacaacagctctggttgccacacagcagttccagcagctgcaggccgctatgcacgacgacctgaaagaggtggaaaagagcatcaccaacctggaaaagtctctgaccagcctgagcgaagtggtgctgcagaacagaagaggcctggacctgctgttcctgaagcgcggaggactgtgcgccttcctgaaagaagagtgttgcctgtacgccgaccacaccggcctcgtcagagattctatggccaagctgagagagagactgagccagagacagaagctgttcgagtcccagcaaggatggttcgagggcctgttcaacaagagcccctggttcaccacactgatcagcacaatcatgggccctctgatcattctgctgctgatcctcctgtttggcccctgcatcctgaacaggctggtgcagttcatcaaggacagaatcagcgtggtgcaggctctggtgctgacccagcagtatcaccagctgaaaaccatcggcgactgcaagagcagagagtga

SEQ ID NO: 6 - protein, 659 aa, Mus musculus
METDTLLLWVLLLWVPGSTGDVALGNSPHQVFNLSWEVTNGDRETVWAITGNHPLWTWWPDLTPDLCMLALHGPSYWGLEYRAPFSPPPGPPCCSGSSDSTSGCSRDCEEPLTSYTPRCNTAWNRLKLSKVTHAHNEGFYVCPGPHRPRWARSCGGPESFYCASWGCETTGRASWKPSSSWDYITVSNNLTSDQATPVCKGNKWCNSLTIRFTSFGKQATSWVTGHWWGLRLYVSGHDPGLIFGIRLKITDSGPRVPIGPNPVLSDRRPPSRPRPTRSPPPSNSTPTETPLTLPEPPPAGVENRLLNLVKGAYQALNLTSPDKTQECWLCLVSGPPYYEGVAVLGTYSNHTSAPANCSVASQHKLTLSEVTGQGLCIGAVPKTHQVLCNTTQKTSDGSYYLAAPTGTTWACSTGLTPCISTTILNLTTDYCVLVELWPRVTYHSPSYVYHQFERRAKYKREPVSLTLALLLGGLTMGGIAAGVGTGTTALVATQQFQQLQAAMHDDLKEVEKSITNLEKSLTSLSEVVLQNRRGLDLLFLKRGGLCAFLKEECCLYADHTGLVRDSMAKLRERLSQRQKLFESQQGWFEGLFNKSPWFTTLISTIMGPLIILLLILLFGPCILNRLVQFIKDRISVVQALVLTQQYHQLKTIGDCKSRE

SEQ ID NO: 7 - DNA, 1161 bp, Gallus gallus
  source 1..1161: mol_type = genomic DNA
atgggctccatcggtgcagcaagcatggaattttgttttgatgtattcaaggagctcaaagtccaccatgccaatgagaacatcttctactgccccattgccatcatgtcagctctagccatggtatacctgggtgcaaaagacagcaccaggacacaaataaataaggttgttcgctttgataaacttccaggattcggagacagtattgaagctcagtgtggcacatctgtaaacgttcactcttcacttagagacatcctcaaccaaatcaccaaaccaaatgatgtttattcgttcagccttgccagtagactttatgctgaagagagatacccaatcctgccagaatacttgcagtgtgtgaaggaactgtatagaggaggcttggaacctatcaactttcaaacagctgcagatcaagccagagagctcatcaattcctgggtagaaagtcagacaaatgggattatcagaaatgtccttcagccaagctccgtggattctcaaactgcaatggttctggttaatgccattgtcttcaaaggactgtgggagaaagcatttaaggatgaagacacacaagcaatgcctttcagagtgactgagcaagaaagcaaacctgtgcagatgatgtaccagattggtttatttagagtggcatcaatggcttctgagaaaatgaagatcctggagcttccatttgccagtgggacaatgagcatgttggtgctgttgcctgatgaagtctcaggccttgagcagcttgagagtataatcaactttgaaaaactgactgaatggaccagttctaatgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgcaagcgtctctgaagaatttagggctgaccatccattcctcttctgtatcaagcacatcgcaaccaacgccgttctcttctttggcagatgtgtttccccttaa

SEQ ID NO: 8 - protein, 386 aa, Gallus gallus
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 9 - DNA, 1623 bp, Homo sapiens
  source 1..1623: mol_type = other DNA
atgtgtcaccaacagctggtcatcagctggttctccctggtgttcctggcctctcctctggtggccatctgggagctgaagaaagacgtgtacgtggtggaactggactggtatcccgatgctcctggcgagatggtggtgctaacctgcgatacacctgaagaggacggcatcacctggacactggatcagtctagcgaggtgctcggctctggcaagaccctgaccatccaagtgaaagagtttggcgacgcaggtcagtacacctgtcacaaaggtggagaagtgctgagccacagcctgctgctgctccacaagaaagaggatggcatttggagtaccgacatcctgaaggatcagaaggagcctaagaacaagaccttcctgagatgcgaggccaagaactatagtggacggttcacatgttggtggctgaccaccatcagcaccgaccttaccttcagcgtgaagagcagcagaggcagcagtgatcctcagggagttacatgtggtgctgctacactgtctgccgaaagagtgagaggtgacaacaaggaatacgagtacagcgtggaatgccaagaggacagcgcttgtccagctgcagaagagtctctgcctatcgaagtgatggtggacgcagtgcacaagctgaagtacgagaactacacctccagcttcttcatcagagacatcatcaagcctgatccacccaagaacctgcagctgaagcctctgaagaacagcagacaggttgaagtgtcctgggagtaccctgacacctggtctacaccacacagctacttcagcctgaccttttgcgtgcaagtgcagggcaagtccaagcgagagaagaaggaccgtgtgttcaccgacaagacaagcgcaaccgtgatctgcagaaagaacgccagcatcagcgtcagagcccaggaccggtactacagcagctcttggagcgaatgggcaagcgtgccatgttctggtggtggaggatctggtggaggtggaagcggaggaggtggatctagaaatctgcctgtggccactcctgatcctggcatgttcccttgtctgcaccacagccagaaccttctgagagcagtgtccaacatgctccagaaggccagacagaccctggaattctacccatgcaccagcgaggaaatcgaccacgaggacatcactaaggataagaccagcaccgtggaagcctgcctgcctctggaactgaccaagaacgagagctgcctgaacagccgtgaaaccagcttcatcaccaacggctcttgcctggcaagcaggaagacctccttcatgatggctctgtgcctgagcagcatctacgaggacctcaagatgtaccaggtggagttcaagaccatgaacgccaagctgctgatggaccctaagcggcagatcttcctggaccagaatatgctggcagtcatcgacgagctgatgcaggcactgaacttcaacagcgagacagtgcctcagaagtctagcctggaggaacccgacttctacaagaccaagatcaagctgtgcatcctgctgcacgccttccgtatcagagccgtgaccatcgacagagtgatgagctacctgaacgcctcctga

SEQ ID NO: 10 - protein, 540 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 11 - DNA, 1719 bp, synthetic construct
  source 1..1719: mol_type = other DNA
atgtgtcaccagcagctggtcatcagctggttcagcctggtgttcctggcctctcctctggtggccatctgggagctgaagaaagacgtgtacgtggtggaactggactggtatcccgatgcacctggcgagatggtggtgctgacctgcgatacacctgaagaggacggcatcacctggacactggaccagtctagcgaggtgctcggctctggcaagaccctgaccatccaagtgaaagagtttggcgacgctggacagtacacctgtcacaaaggtggagaagtgctgagccacagcctgctgctgctccacaagaaagaggatggcatttggtccaccgacatcctgaaggaccagaaagagcccaagaacaagaccttcctgagatgcgaggccaagaactactccggacggttcacatgttggtggctgaccaccatcagcaccgacctgacattcagcgtgaagagtagcagaggcagcagtgatcctcagggagttacatgtggagcagctacactgtctgccgaaagagtgagaggtgacaacaaagaatacgagtacagcgtggaatgccaagaggatagtgcctgtccagcagcagaagagtctctgcctatcgaagtgatggtggacgctgtgcacaagctgaagtacgagaactacacatccagcttcttcatccgagacatcatcaaaccagatcctcccaagaatctgcagctgaagcctctgaagaacagcagacaagtggaagtgtcctgggagtacccagacacctggtctacacctcacagctacttctccctgaccttttgcgtgcaagtgcagggcaagtccaagagagagaagaaggacagagtcttcaccgacaagacatctgccaccgtgatctgcagaaagaacgccagcatcagcgtcagagcccaggaccggtactacagcagctcttggagcgaatgggcaagcgtgccatgttctggtggtggaggatctggaggaggtggaagcggtggaggaggatctagacctgttagcctgcagtgcagactgagcatgtgcccagctagatctctcctgctggttgccacactggtgctcctggatcatctgagcctggccagaaacctgccagtggccacgcctgatcctggcatgtttccttgtctgcaccacagccagaacctgctgagagccgtttccaacatgctgcagaaggccagacagaccctggaattctacccatgcaccagcgaggaaatcgaccacgaggacattaccaaggataagaccagcaccgtggaagcctgcctgcctctggaactgaccaagaacgagagctgcctgaacagccgtgaaaccagcttcatcaccaacggctcttgccttgcctccaggaagacctccttcatgatggcactgtgcctgagcagcatctacgaggacctcaagatgtaccaagtggagttcaagaccatgaacgccaagctgctgatggatcccaagagacagatcttccttgatcagaacatgctggctgtgatcgacgagctgatgcaggcactgaacttcaacagcgagacagtgcctcagaagtctagcctggaagaacccgacttctacaagaccaagatcaagctgtgcatcctgctgcacgccttccgtatcagagccgtgaccatcgacagagtgatgagctacctgaacgcctcctga

SEQ ID NO: 12 - protein, 572 aa, synthetic construct
MCHQQLVISWFSLVFLASPLVAIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRPVSLQCRLSMCPARSLLLVATLVLLDHLSLARNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 13 - DNA, 768 bp, Homo sapiens
  source 1..768: mol_type = genomic DNA
atggaatacgccagcgacgcctctctggaccctgaagctccttggcctccagctcctagagccagggcttgtagagtgctgccttgggctcttgtggctggacttctgcttctgttgctcctggctgctgcctgcgcagtgtttcttgcttgtccatgggctgtgtcaggagccagagcatctcctggatctgccgcttctcccagactgagagagggacctgaactgagccctgatgatcctgctggactgctcgacctgagacagggcatgtttgcccagctggtggcccagaatgtgctgctgattgatggccctctgagctggtacagcgatcctggacttgctggcgttagcctgactggaggcctgagctacaaggaggacaccaaagaactggtggtggccaaggctggcgtgtactacgtgttctttcagctggaactgcggagagtggtggcaggcgaaggatctggatccgtgtctctggcactgcatctgcagcctctgagatctgctgctggtgcagctgccctggctctgacagttgatctgcctcctgcctccagcgaagccagaaacagcgcctttggcttccaaggcagactgctgcacctgtctgctggccagagactgggagtgcacctccacacagaagcaagagcaagacacgcctggcagcttacacaaggcgctacagtgctgggcctgttcagagtgacacctgagattccagctggcttgccatctcctcgcagcgagtaatga

SEQ ID NO: 14 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

(19) United States
(12) Patent Application Publication
(10) Pub. No.: US 2025/0049903 A1
(43) Pub. Date: Feb. 13, 2025

(54) Therapy for Modulating Immune Response with Recombinant MVA Encoding IL-12

(71) Applicant: Bavarian Nordic A/S, Hellerup (DK)

(72) Inventors: Cigdem Atay Langbein, Munich (DE); André Riedl, Schweitenkirchen (DE); Markus Kalla, Penzberg (DE); Maria Hinterberger, Sauerlach (DE); José Medina Echeverz, Munich (DE); Pedro Berraondo López, Pamplona (ES)

(73) Assignee: Bavarian Nordic A/S, Hellerup (DK)

(21) Appl. No.: 18/722,828

(22) PCT Filed: Dec. 23, 2022
(86) PCT No.: PCT/EP2022/087718

§ 371 Date: Jun. 21, 2024

Related U.S. Application Data
(60) Provisional application No. 63/293,170, filed on Dec. 23, 2021.

(51) Int. Cl. A61K 39/00; A61P 35/00; C12N 15/86

(52) U.S. Cl. CPC A61K 39/0011; A61K 39/00114; A61P 35/00; C12N 15/86; A61K 2039/5256; A61K 2039/54; C12N 2710/24143

(57) ABSTRACT

The invention relates to a composition and related methods for enhancing the immune response and/or decreasing the size or growth rate of a tumor volume in a subject. The composition comprises a recombinant MVA encoding IL-12 and, optionally, a Tumor Associated Antigen (“TAA”). In some embodiments, the MVA also encodes 4-1BBL and/or a second MVA encoding 4-1BBL is coadministered to the subject. In some embodiments of the invention, methods comprise injecting these MVAs intratumorally. In some embodiments, the recombinant MVAs are injected intraperitoneally to stimulate an immune response to peritoneal tumors.

### FIELD OF THE INVENTION

The present invention relates to a therapy for modulating the immune response of a subject comprising injection of the subject with a recombinant Modified Vaccinia Ankara virus (“MVA”) comprising a nucleic acid encoding IL-12. The invention thus relates also to compositions comprising a recombinant modified vaccinia Ankara virus (MVA) encoding IL-12, and their use in stimulating an immune response to Tumor Associated Antigens (TAAs). In some embodiments, the subject is also injected with a recombinant MVA comprising a nucleic acid encoding 4-1BBL, or the subject is injected with a recombinant MVA comprising both a nucleic acid encoding IL-12 and a nucleic acid encoding 4-1BBL.

### BACKGROUND OF THE INVENTION

Recombinant poxviruses have been used as immunotherapy vaccines against infectious organisms and, more recently, against tumors (Mastrangelo et al. (2000) J Clin Invest. 105(8):1031-1034). One poxviral strain that has proven useful as an immunotherapy vaccine against infectious disease and cancer is the Modified Vaccinia Ankara (MVA) virus (sometimes referred to simply as “MVA”). MVA was generated by 516 serial passages on chicken embryo fibroblasts of the Ankara strain of vaccinia virus (CVA) (for review see Mayr et al. (1975) Infection 3: 6-14). As a consequence of these long-term passages, the genome of the resulting MVA virus had about 31 kilobases of its genomic sequence deleted, and was described as highly host cell restricted for replication to avian cells (Meyer et al. (1991) J. Gen. Virol. 72: 1031-1038). It was shown in a variety of animal models that the resulting MVA was significantly avirulent (Mayr & Danner (1978) Dev. Biol. Stand. 41: 225-34).

Strains of MVA having enhanced safety profiles for the development of safer products, such as vaccines or pharmaceuticals, have been described (see International PCT publication WO2002042480; see also, e.g., U.S. Pat. Nos. 6,761,893 and 6,913,752, all of which are hereby incorporated by reference herein). Such variants are capable of reproductive replication in non-human cells and cell lines, especially in chicken embryo fibroblasts (CEF), but are replication incompetent in human cell lines, in particular including HeLa, HaCat and 143B cell lines. Such strains are also not capable of reproductive replication in vivo, for example, in certain mouse strains, such as the transgenic mouse model AGR 129, which is severely immune-compromised and highly susceptible to a replicating virus (see, e.g., U.S. Pat. No. 6,761,893). Such MVA variants and derivatives, including recombinants, referred to as “MVA-BN,” have been described (see International PCT publication WO2002/042480; see also, e.g., U.S. Pat. Nos. 6,761,893 and 6,913,752).

Poxviral vectors encoding tumor-associated antigens (TAAs) have been shown to successfully reduce tumor size as well as increase overall survival rate when administered to cancer patients (see, e.g., WO 2014/062778). It has been demonstrated that when a cancer patient is administered a poxviral vector encoding a TAA such as HER2, CEA, MUC1, and/or Brachyury, a robust and specific T-cell response is generated by the patient to fight the cancer (Id.; see also, Guardino et al. ((2009) Cancer Res. 69 (24), doi 10.1158/0008-5472.SABCS-09-5089), Heery et al. (2015) JAMA Oncol. 1: 1087-95).

One type of TAA that was found to be expressed on many cancer and tumor cells are Endogenous Retroviral (ERV) proteins. ERVs are remnants of former exogenous forms that invaded the germ line of the host and have since been vertically transmitted through a genetic population (see Bannert et al. (2018) Frontiers in Microbiology, Volume 9, Article 178). ERV-induced genomic recombination events and dysregulation of normal cellular genes have been documented to have contributory effects to tumor formation (Id.). Further, there is evidence that certain ERV proteins have oncogenic properties (Id.). ERVs have been found to be expressed in a large variety of cancers including, e.g., breast, ovarian, melanoma, prostate, and pancreatic cancer, as well as lymphoma (see, e.g., Atterman et al. (2018) Ann. Oncol. 29: 2183-91).

In addition to their effectiveness with TAAs, poxviruses such as MVA have been shown to have enhanced efficacy when combined with a CD40 agonist such as CD40 Ligand (“CD40L”) (see WO 2014/037124) or with a 4-1BB agonist such as 4-1BB Ligand (“4-1BBL”) (Spencer et al. (2014) PLoS One 9: e105520; see also WO2020104531).

4-1 BB/4-1BBL is a member of the TNFR/TNF superfamily. 4-1 BBL is a costimulatory ligand expressed in activated B cells, monocytes and DCs. 4-1BB is constitutively expressed by natural killer (NK) and natural killer T (NKT) cells, Tregs, and several innate immune cell populations, including DCs, monocytes and neutrophils. Interestingly, 4-1BB is expressed on activated T cells, but not resting T cells (Wang et al. (2009) Immunol. Rev. 229: 192-215). 4-1BB ligation induces proliferation and production of interferon gamma (IFN-γ) and interleukin 2 (IL-2), and enhances T cell survival through the upregulation of antiapoptotic molecules such as Bcl-xL (Snell et al. (2011) Immunol. Rev. 244: 197-217). Importantly, 4-1BB stimulation enhances NK cell proliferation, IFN-γ production and cytolytic activity through enhancement of Antibody-Dependent Cell Cytotoxicity (ADCC) (Kohrt et al. (2011) Blood 117: 2423-32).

The 4-1BB/4-1BBL axis of immunity has been explored using various immunotherapeutic strategies. As an example, autologous transfer of Chimeric Antigen Receptor (CAR) T cells showed clinical benefit in large B cell lymphomas and were approved by the FDA in 2017. Patient autologous T cells were transduced with CARs that combined an extracellular domain derived from a tumor-specific antibody, the CD3ζ intracellular signaling domain, and the 4-1BB costimulatory motif. The addition of 4-1BB was crucial for in vivo persistence and antitumor toxicity of CAR T cells (Song et al. (2011) Cancer Res. 71: 4617e27). Antibodies targeting 4-1BB are currently being investigated.

Several studies have shown that agonistic antibodies targeting the 4-1BB/4-1BBL pathway show anti-tumor activity when utilized as a monotherapy (Palazón et al. (2012) Cancer Discovery 2: 608-23), and agonistic antibodies targeting 4-1BB (Urelumab, BMS; Utomilumab, Pfizer) were being tested for clinical use. However, in recent years, studies that have combined 4-1BBL with other therapies have shown varied success. For example, when mice with preexisting MC38 (murine adenocarcinoma) tumors, but not B16 melanoma tumors, were administered with antibodies to CTLA-4 and anti-4-1BB, significant CD8+ T cell-dependent tumor regression was observed, together with long-lasting immunity to these tumors (Kocak et al. (2006) Cancer Res. 66: 7276-84). In another example, treatment with anti-4-1BB (Bristol-Myers Squibb (BMS)-469492) led to only modest regression of M109 tumors, but significantly delayed the growth of EMT6 tumors (Shi and Siemann (2006) Anticancer Res. 26: 3445-54).

IL-12 is a type 1 cytokine that has also been investigated as a monotherapy treatment for cancer, but early clinical trials found dose-limiting toxicity (see, e.g., Nguyen et al. (2020) Front. Immunol. 11: 575597). For example, “[i]n one phase II trial, a maximal dose of 0.5 μg/kg/day resulted in severe side effects in 12 out of 17 enrolled patients and the deaths of two patients,” even though the same dose had been well-tolerated in an earlier phase I study (Id., citing Jenks (1996) J. Nat'l. Cancer Inst. 88: 576-7). IL-12 was also administered at lower doses that were more readily tolerated, but showed limited efficacy (Id.). These studies generally utilized systemic (i.e., intravenous) administration or subcutaneous administration; however, in one study that examined them, posttreatment metastatic lesions were shown to have undergone infiltration by CD8+ T cells. (Id.)

The tumor microenvironment is composed of a large variety of cell types, from immune cell infiltrates to cancer cells, extracellular matrix, endothelial cells, and other cellular components and factors that influence tumor progression. This complex and entangled equilibrium changes not only from patient to patient, but within lesions in the same subject (Jiménez-Sánchez et al. (2017) Cell 170(5): 927-938). Stratification of tumors based on Tumor Infiltrating Lymphocytes (TIL) and Programmed Death Ligand 1 (PD-L1) expression emphasizes the importance of an inflammatory environment to achieve objective responses against cancer (Teng et al. (2015) Cancer Res. 75(11): 2139-45). Pan-cancer analysis of gene expression profiles from the Cancer Genome Atlas (TCGA) supports that a tumor inflammation signature correlates with objective responses to immunotherapy (Danaher et al. (2018) J. Immunother. Cancer 6(1): 63).

In recent years, attempts to improve cancer therapies using different routes of administration of vaccines have evaluated subcutaneous injection as well as intravenous administration. For example, it was demonstrated that an intravenous administration of an MVA vaccine encoding a heterologous antigen was able to induce a strong specific immune response to the antigen (see WO 2014/037124). Further, increased and enhanced immune responses were generated when the MVA vaccine included CD40L.

Intratumoral administration of MVA vaccines has been reported. It was found that intratumoral injections of MVA expressing GM-CSF and immunization with DNA vaccine prolonged the survival of mice bearing HPV16 E7 tumors (Nemeckova et al. (2007) Neoplasma 54: 4). Other studies of intratumoral injection of MVA were unable to demonstrate inhibition of pancreatic tumor growth (White et al. (2018) PLoS One 13(2): e0193131). Intratumoral injection of heat-inactivated MVA induced antitumor immune responses that were dependent on the generation of danger signals, type I interferon, and antigen cross-presentation by dendritic cells (Dai et al. (2017) Sci. Immunol. 2(11): eaal1713).

There is clearly a substantial unmet medical need for additional cancer treatments, including active immunotherapies and cancer vaccines. Additionally, there is a need for therapies that can induce enhanced immune responses in multiple areas of a patient's immune response. In many aspects, the embodiments of the present disclosure address these needs by providing vaccines and therapies that increase the immune response to tumors and improve the cancer treatments currently available.

### BRIEF SUMMARY OF THE INVENTION

The present invention relates to a therapy for modulating the immune response of a subject comprising injection of the subject with a recombinant Modified Vaccinia Ankara virus (“MVA”) comprising a nucleic acid encoding IL-12 and a nucleic acid encoding a Tumor Associated Antigen (TAA). The invention thus relates also to compositions comprising a recombinant Modified Vaccinia Ankara virus (MVA) encoding IL-12 and their use in stimulating an immune response to TAAs. In some embodiments, recombinant MVAs of the invention further comprise a nucleic acid encoding 4-1BBL. Thus, in some embodiments, recombinant MVAs of the invention comprise a nucleic acid encoding a TAA, a nucleic acid encoding IL-12, and a nucleic acid encoding 4-1BBL. In other embodiments, a composition of the invention comprises a mixture of recombinant MVAs, one of which encodes IL-12 and one of which encodes 4-1BBL, wherein at least one of the MVAs also encodes a TAA.

The invention also provides methods of use and/or treatment with the recombinant MVAs in which one or more recombinant MVAs of the invention is administered intratumorally, intravenously, or intraperitoneally to a subject having tumors. In some embodiments, the recombinant MVAs of the invention are used to prepare a medicament to increase the immune response of a subject to a tumor. In some embodiments, the recombinant MVAs of the invention are used to prepare a medicament for intratumoral injection to increase the immune response of the subject to the injected tumor; in some embodiments, injection of the medicament into the tumor may decrease the size and/or growth rate of the injected tumor and may also decrease the size and/or growth rate of other tumors that were also present in the subject but that were not intratumorally injected with the medicament (i.e., with the recombinant MVAs).

In some embodiments, the subject has peritoneal tumors and the medicament is for intraperitoneal injection, whereby an immune response to peritoneal tumors is stimulated or enhanced.

In some embodiments, the invention provides an intravenously or intratumorally administered recombinant MVA comprising a nucleic acid encoding a TAA and a nucleic acid encoding IL-12. In some embodiments, the invention provides an intratumorally and/or intravenously administered recombinant MVA comprising nucleic acids encoding a TAA, IL-12, and 4-1BBL (CD137L); in other embodiments, the invention provides a combination of recombinant MVAs, one of which encodes IL-12, one of which encodes 4-1BBL, and at least one of which also encodes a TAA. This combination of recombinant MVAs is administered to a subject so that for some period of time, they are present in the subject together. In some embodiments, the recombinant MVA comprises nucleic acids encoding a TAA, IL-12, and 4-1BBL, and is administered intraperitoneally to a subject multiple times, such as at least 2 times or at least 3 times, resulting in the induction of an immune response against tumors in the subject and/or a decrease in growth or size of tumors in the subject.

The aspects and advantages of the various embodiments of the invention are described in more detail below. The accompanying drawings, which are incorporated in and constitute a part of this specification, illustrate one or more embodiments of the invention and, together with the description, serve to explain the principles of the invention.

### BRIEF DESCRIPTION OF THE DRAWINGS

FIG. 1 shows that local MVA-Gp70-IL12 injection results in systemic inflammation. C57BL/6 mice were inoculated subcutaneously with 5×10⁵ B16.F10 cells. When tumors were above 60 mm³ in volume, mice were grouped and injected intratumorally (IT) with either saline or increasing concentrations of either MVA-Gp70 or MVA-Gp70-IL12 (TCID₅₀ virus; 5×10⁶, 5×10⁷, or 2×10⁸ TCID₅₀ of each recombinant MVA shown in FIG. 1). Six hours after IT injection, mice were bled and sera was collected for cytokine/chemokine analysis by Luminex. Data are shown as Mean±SEM.

FIG. 2 shows that increasing doses of IT MVA-Gp70-IL12 induces rejection of poorly immunogenic B16.F10 melanomas. C57BL/6 mice were inoculated subcutaneously with 5×10⁵ B16.F10 cells. When tumors were above 60 mm³ in volume, mice were grouped and injected intratumorally (IT; Day 0) with either saline or increasing concentrations of either MVA-Gp70 or MVA-Gp70-IL12 (TCID₅₀ virus; 5×10⁶, 5×10⁷, or 2×10⁸ TCID₅₀ of each recombinant MVA shown in FIG. 2). Mice received additional IT (“boost”) immunizations at days 5 and 8 (vertical dotted lines). Tumors were measured at regular intervals.

FIG. 3 shows that local immunization of MVA-Gp70-IL12 combined with MVA-Gp70-4-1BBL induces complete rejection of poorly immunogenic B16.F10 melanomas. C57BL/6 mice were inoculated subcutaneously with 5×10⁵ B16.F10 cells. When tumors were above 60 mm³ in volume, mice were grouped and injected intratumorally (IT) with either saline, MVA-Gp70, MVA-Gp70-IL12, or the combination of MVA-Gp70-IL12 with MVA-Gp70-4-1BBL (Day 0). All recombinant MVAs and combinations were administered at a dosage of 5×10⁷ TCID₅₀. Mice received additional (“boost”) IT immunizations at days 5 and 8 (vertical dotted lines). Tumors were measured at regular intervals.

FIG. 4 shows that IT injection with MVA-Gp70-IL12 alone or combined with MVA-Gp70-4-1BBL induces rejection of MC38 colon carcinomas. C57BL/6 mice were inoculated subcutaneously with 5×10⁵ MC38 cells. When tumors were above 60 mm³ in volume, mice were grouped and injected intratumorally (IT) with either saline, MVA-Gp70, MVA-Gp70-4-1BBL, MVA-Gp70-IL12, or a combination of both 4-1BBL and IL12-expressing MVAs. All recombinant MVAs and combinations were administered at a dosage of 5×10⁷ TCID₅₀. Mice received additional (“boost”) IT immunizations at days 5 and 8 (vertical dotted lines). Tumors were measured at regular intervals. Number of cured mice is indicated in the lower right corner.

FIG. 5 shows that IT injection with MVA-Gp70-IL12 combined with MVA-Gp70-4-1BBL induces peripheral antigen-specific CD8⁺ T cell responses. Mice were treated as described in FIG. 4. Three days after the last IT immunization, mice were bled and blood samples subjected to peptide restimulation. The figure shows percentage of CD8⁺ T cells among alive counterparts and CD44+ IFN-γ⁺TNF-α⁺ cells as a percentage of CD8⁺ T cells after restimulation with the immunodominant Gp70 antigen p15E. Data are expressed as Mean±SEM.

FIGS. 6A and 6B: Antitumor effect of treated or untreated tumors after IT MVA-Gp70-IL12sc or MVA-Gp70-IL12+MVA-Gp70-4-1BBL treatment of MC38 bilateral tumors. C57BL/6 mice were inoculated subcutaneously with 5×10⁵ MC38 cells on the right flank and 2×10⁵ on the left flank. When right flank tumors were above 60 mm³ in volume, mice were grouped and received intratumoral (IT) injections with either saline, MVA-Gp70, MVA-Gp70-4-1BBL, MVA-Gp70-IL12 or a combination of both 4-1BBL and IL12-expressing MVAs. All viruses and combination were used at a TCID₅₀ of 5×10⁷. Mice received additional (“boost”) IT immunizations at days 5 and 8 (vertical dotted lines). Tumors were measured at regular intervals. Number of cured mice is indicated in the lower right corner.

FIGS. 7A and 7B: Rejection of tumor by cured mice after IT MVA-Gp70-IL12 or MVA-Gp70-IL12+MVA-Gp70-4-1BBL, after rechallenge. Naïve C57BL/6 mice and C57BL/6 mice that were cured of MC38 tumors were rechallenged with MC38 tumor cells in the opposite flank where the primary tumor was placed. Tumor growth was measured at regular intervals. Tumor free survival of mice (FIG. 7A) upon time and percentage of tumor antigen-specific CD8⁺ T cells in blood pre and post tumor rechallenge (FIG. 7B). Data in FIG. 7B are expressed as Mean±SEM.

FIGS. 8A, 8B, and 8C: MVA-IL12 treatment induces adaptive-specific immune responses against MC38 colorectal peritoneal carcinomatosis. C57BL/6 mice (n=4) were challenged intraperitoneally (“i.p.”) with 5×10⁵ MC38 tumor cells. After seven days of tumor challenge, they were treated with a single dose of 5×10⁷ TC_{ID50} (200 μl volume) MVA-mock as a control group or MVA-IL-12 by intraperitoneal (i.p.) administration. Specific immune response against MC38 tumor was analyzed after seven days MVA's inoculation (i.e., Day 14). The spleen was processed, and 5×10⁵ cells/well were incubated for 24 hours with tumor-associated KSPWFTTL peptide (for stimulation of mouse MC38-specific CD8⁺ T cells), irradiated (20,000 rads) 5×10⁴ MC38 tumor cells, or without-antigen as non-specific response. Values are represented as mean±SEM. *p<0.05 (unpaired t test). FIG. 8B: CD8 lymphocytes were analyzed by flow cytometry in both the spleen and the peritoneal wash. MVA-IL-12 increased antigen-specific CD8⁺ cells. FIG. 8C: MVA-IL-12 also increased the percentage of lytic CD107⁺CD8⁺ cells capable of producing both IFN-γ and TNF-α both locally and systemically (FIG. 8C; see Example 8).

FIGS. 9A, 9B, and 9C: Intraperitoneal MVA-IL-12 treatment cured all MC38 peritoneal carcinomatosis bearing mice and showed complete protection after tumor rechallenge. C57BL/6 mice (6 per group) were challenged i.p. with 5×10⁵ MC38 tumor cells. FIG. 9A: after seven days of tumor challenge, mice were treated with a single dose of 5×10⁷ TC_{ID50} (200 μl volume) MVA.mock as a control group or MVA-IL-12 by intraperitoneal administration and untreated group. Survival was monitored daily. FIG. 9B: mice (n=6) that rejected MC38 peritoneal carcinomatosis after MVA-IL-12 treatment (“survivors”) were rechallenged i.p. with 5×10⁵ MC38 tumor cells. A naïve group was included as control group (n=5). Survival was monitored daily. Values are represented as Kaplan-Meier method ****p<0.0001 (log-rank test). FIG. 9C: Monoclonal antibodies against CD8 T lymphocytes or NK cells were used concomitantly with MVA.scIL-12 (see Example 9). The antitumor effect was greatly diminished by CD8 depletion, although not completely abolished. The depletion of NK cells alone did not have any impact on overall survival. These results demonstrate that CD8 T cells are required for the antitumor effect but act together with other immune cells to achieve maximum efficacy.

FIGS. 10A, 10B, 10C, 10D, 10E, 10F, 10G, and 10H: MVA-IL-12 i.p. is more effective and less toxic than the i.v. route to treat peritoneal carcinomatosis (see Example 10). C57BL/6 mice (6 per group) were challenged i.p. with 5×10⁵ MC38 tumor cells. After seven days of tumor challenge, they were treated with a single dose of 5×10⁷ TC_{ID50} (200 μl volume) MVA.mock i.p., MVA-IL-12 by intraperitoneal (i.p.) or intravenous (i.v.) administration. FIG. 10A: weight (in grams) was monitored daily after MVA's treatment during the following 10 days (top panel), but showed greater differences between the groups at 48 and 96 hours (bottom panel). These results demonstrated that i.v. administration dramatically reduced the antitumor activity of MVA-IL-12 while increasing the toxicity associated with the systemic expression of IL-12: weight loss was observed two and four days after i.v. administration, but not when intraperitoneal (i.p.) administration was used. FIG. 10B: survival of mice was determined daily. Weights are represented as mean±SEM. **p<0.01 (unpaired t test). Survival is represented according to the Kaplan-Meier method; ****p<0.0001 (log-rank test). The results showed that survival was significantly lower with i.v. administration of MVA-IL-12 in comparison to i.p. administration. Other effects of i.v. administration of MVA included hematologic toxicity, as reflected by reduced numbers of platelets 24 h and 72 h after administration in comparison to i.p. administration (FIG. 10C). The i.p. and i.v. route decreased the number of circulating white blood cells (FIG. 10D). MVA-IL-12 administered by the i.v. route induced very high levels of both scIL-12 and IFN-γ systemically (as measured in serum), which may explain the previous toxic effects described (FIG. 10E). However, i.p. administration induced a dramatic increase in the concentrations of IL-12 and IFN-γ in peritoneal wash (FIG. 10F). I.p. administration also increased the percentage of tumor-specific CD8+ T cells systemically as well as in the peritoneum, while i.v. administration did not increase specific cell levels in the peritoneum and very variably increased levels in the spleen (FIG. 10G). Finally, using the ELISpot technique, we showed that i.p. administration was able to generate lymphocytes specific for both the particular antigen and entire tumor cells, while the i.v. route was not able to increase these levels in spleen (FIG. 10H).

FIGS. 11A, 11B, 11C, 11D, 11E, and 11F: FIG. 11A shows schematic diagrams of recombinant MVAs. FIG. 11B: Mouse splenocytes incubated with MVA-IL-12 induced the release of detectable amounts of scIL-12 into the supernatants in a dose-dependent manner, while MVA alone did not induce IL-12 production. FIG. 11C: MVA-IL-12 induced IL-12 expression, with maximum levels obtained 6 hours after administration, while MVA alone did not induce expression (left panel). IFN-γ induced by IL-12 was delayed, and maximum levels were detected 48 h after vector administration (right panel). FIG. 11D: MVA-IL-12 induced IL-12 expression in both peritoneal wash and in serum at 6 h after administration, while IL-12 was not detected after administration of MVA alone. FIG. 11E: MVA-IL-12 (here, MVA-scIL-12) infected tumor lines and produced IL-12 in supernatants for MC38, CT26, and ID8.Vegf. FIG. 11F: supernatants from MC38 as shown in FIG. 11E exhibited immunostimulatory activity by inducing IFN-γ when incubated with splenocytes (see Example 1).

FIG. 12A: Mice were implanted intraperitoneally with CT26 colon cancer cells (2×10⁶ cells) or ID8.Vegf/GFP ovarian cancer cells (1×10⁷ cells) (=Day 0) (see Example 11). Mice were then inoculated with one dose of MVA or MVA-IL-12 (see Example 11); survival is shown in FIG. 12A for mice implanted with CT26 cancer cells (left graph) and for mice implanted with ID8.Vegf/GFP cells (right graph). FIG. 12B shows survival for subjects implanted with either CT26 cancer cells (left graph) or ID8.Vegf/GFP cells (right graph) and then inoculated three times with MVA or MVA-IL-12 (see Example 11).

FIG. 13A: Experiments were conducted to compare the intraperitoneal (i.p.) and intravenous (i.v.) routes of administration to the intratumoral (i.t.) route. Mice were injected subcutaneously (s.c.) with MC38 cells (5×10⁵ cells). MVA-IL-12 was administered to subjects intratumorally seven days later. This treatment delayed the death of all mice and achieved a cure rate of approximately 30% (FIG. 13A), but these results were inferior to those obtained with i.p. administration in previous experiments using MC38 cells. FIG. 13B: further experiments were conducted to explore local and systemic effects of s.c. and i.p. administration of MVA-IL-12 (see Example 12). FIG. 13D: Mice that eradicated the tumor in the peritoneum after i.p. treatment with MVA-IL-12 were able to eliminate MC38 cells injected subcutaneously in a rechallenge. FIG. 13E: Of mice that eradicated the subcutaneous tumor after i.t. administration of MVA-IL-12, only 65% were able to reject a rechallenge with MC38 cells administered i.p. FIG. 13C: Experiments were conducted to investigate the ability of a peritoneal tumor to initiate a systemic immune response (see Example 12).

FIGS. 14A, 14B, 14C, and 14D: FIG. 14A shows bioluminescence following i.p. administration to subjects of MVA encoding luciferase. FIG. 14B shows localization of luciferase following i.p. or i.v. administration to subjects of MVA encoding luciferase (see Example 13). FIG. 14C shows comparison of transcriptomic profile of subjects treated with MVA versus those treated with MVA-IL-12, and FIG. 14D shows that different transcriptomic profiles resulted from treatment of subjects with MVA-IL-12 intraperitoneally (i.p.) and intravenously (i.v.) (see Example 13).

FIGS. 15A, 15B, and 15C show dose-dependent effects of IT administration of MVA-Gp70-4-1BBL-IL12 in B16.F10 melanoma-bearing mice (see Example 14). FIG. 15A shows tumor mean diameter for all groups. FIG. 15B shows mouse survival for various groups. FIG. 15C shows the percentage of CD8⁺ T cells as percentage of live cells and percentage of CD44⁺ IFNγ⁺ expressing CD8⁺ T cells; these cells were restimulated with the immunodominant Gp70 antigen p15E. Data are shown as Mean±SEM (5 mice/group; see Example 14).

FIGS. 16A, 16B, 16C, 16D, 16E, and 16F show that intratumoral (i.t.) administration of MVA-Gp70-4-1BBL-IL12 induces a systemic tumor-specific immune response in B16.F10 bilateral tumor bearing mice (Example 15). FIG. 16A shows the bilateral tumor model and i.t. immunization scheme. FIG. 16B shows treated and untreated tumor mean diameters are indicated for mice inoculated with PBS, while FIG. 16C shows tumor mean diameters for mice inoculated with MVA-Gp70-4-1BBL-IL12. FIG. 16D shows tumor mean volume for all treatment groups, and FIG. 16E shows mouse survival for all treatment groups. FIG. 16F shows the percentage of CD8⁺ T cells among live cells and the percentage of CD44⁺ IFNγ⁺ and CD44⁺IFNγ⁺TNFα⁺ among CD8⁺ T cells. Data are shown as Mean±SEM, 5-7 mice per group. (Example 15).

FIGS. 17A, 17B, 17C, 17D, 17E, and 17F: Repetitive local administration of MVA-Gp70-4-1BBL-IL12 promotes a systemic tumor-specific immune response, which is not dependent on NK cells (Example 16). C57BL/6 mice received 5×10⁵ and 2×10⁵ MC38 tumor cells into the right and left flank via s.c. injection. After 14 days, mice were grouped according to the size of tumors on the right side and received either anti-NK1.1 antibody or its isotype control IgG2a (200 μg/mouse) via i.p. injection. One day later, mice received PBS or 5×10⁷ TCID₅₀ MVA-Gp70-4-1BBL-IL12 via i.t. injections of the right tumor. This day was designated “Day 0” and treatment was repeated on Day 4 and Day 7 (vertical dotted lines). Tumor growth was measured at regular intervals. FIG. 17A shows the bilateral tumor model and MVA i.t. immunization scheme. Treated and untreated tumor mean diameters are shown in FIG. 17B (PBS (i.t.)+IgG2a (i.p.)), FIG. 17C (PBS (i.t.)+anti-NK1.1(i.p.)), FIG. 17D (MVA-Gp70-4-1BBL-IL12 (i.t.)+IgG2a (i.p.)), and FIG. 17E (MVA-Gp70-4-1BBL-IL12 (i.t.)+anti-NK1.1 (i.p.)) treated mice. FIG. 17F shows the tumor mean volume for all animal groups. Data are shown as Mean±SEM; 10 mice/group.

FIGS. 18A, 18B, 18C, 18D, 18E, 18F, and 18G: Repetitive local administration of MVA-Gp70-4-1BBL-IL12 induces a strong tumor-specific immune response, which is partly dependent on CD8⁺ cells (Example 17). C57BL/6 mice received 5×10⁵ MC38 tumor cells into the right flank via s.c. injection. After 15 days, mice were grouped according to the size of tumors and received either anti-CD8 antibody or its isotype control rat-IgG2b (100 μg/mouse) via i.p. injection. One day later (“Day 0”), mice received PBS or 5×10⁷ TCID₅₀ MVA-Gp70-4-1BBL-IL12 via i.t. injections of the tumor. This treatment was repeated on day 6 and 10 (vertical dotted lines). Cells were stained with fluorochrome-labelled antibodies and analyzed via flow cytometry, and tumor growth was measured at regular intervals. FIG. 18A: percentage of CD8⁺ T cells among live cells after the 1^{st} i.p. injection is shown. Tumor mean diameters are indicated for FIG. 18B ((PBS (i.t.)+rat-IgG2b (i.p.)), FIG. 18C (PBS (i.t.)+anti-CD8 (i.p.)), FIG. 18D (MVA-Gp70-4-1BBL-IL12 (i.t.)+rat-IgG2b (i.p.)) and FIG. 18E (MVA-Gp70-4-1 BBL-IL12 (i.t.)+anti-CD8 (i.p.) treated mice). Tumor mean volume (FIG. 18F) and mouse survival (FIG. 18G) is shown for all treatment groups. Data are shown as Mean±SEM; 10 mice/group.

FIGS. 19A and 19B: Repetitive local administration of MVA-TAA-4-1BBL-IL12 induces tumor-specific memory response in treated MC38 mice (Example 18). Naïve C57BL/6 mice and mice previously cured of tumors by injections of recombinant MVA (MVA-TAA-4-1BBL-IL12) received 5×10⁵ MC38 tumor cells by s.c. injection into the left flank (which in the case of the cured mice had not been previously injected). Peripheral blood was withdrawn one day before and fourteen days after the tumor rechallenge, stained with fluorescently labelled antibodies, and analyzed by flow cytometry. Tumor growth was measured at regular intervals. FIG. 19A: Tumor mean diameters. FIG. 19B: Percentage of antigen specific CD8⁺ T cells before and after tumor rechallenge is indicated. Data are shown as Mean±SEM; 3-10 mice/group.

It is to be understood that both the foregoing Summary and the following Detailed Description are exemplary and explanatory only and are not restrictive of the invention, as claimed.

### DETAILED DESCRIPTION OF THE INVENTION

The recombinant MVAs and methods of the present invention increase and enhance multiple aspects of a subject's immune response to one or more tumors. In various aspects, the present invention demonstrates that when a recombinant MVA comprising a nucleic acid encoding at least one tumor-associated antigen (TAA) and a nucleic acid encoding IL-12 is administered intratumorally to a subject, there is an increased anti-tumor effect realized in the subject. As described in more detail herein, this anti-tumor effect includes, for example, a decrease in tumor size/volume, a decrease in tumor growth rate, increased overall survival rate, an enhanced CD8+ T cell response to the TAA, and enhanced inflammatory responses such as increased cytokine production in the tumor and even in some embodiments systemically in the subject, as compared to an administration of a recombinant MVA by itself.

In further embodiments of the invention, a recombinant MVA encoding 4-1BB Ligand (also referred to herein as 41BBL, 4-1BBL, or CD137L) when administered in combination with a recombinant MVA encoding IL-12 (wherein at least one of the recombinant MVAs also encodes a TAA) further increases the effectiveness of and/or enhances the immune response and therefore the treatment of a subject having tumors.

Recombinant modified vaccinia Ankara (MVA) virus as used herein (also “recombinant MVA” or “rMVA”) refers to an MVA comprising at least one polynucleotide encoding a heterologous gene, such as, for example, a tumor associated antigen (TAA). By “in combination” is intended that one or more treatments is present at the same time in a subject. For example, a combination comprising recombinant MVA encoding IL-12 and a recombinant MVA encoding 4-1BBL are present in the subject at the same time, even though they may be administered to the subject at different times and/or by different routes of administration. Thus, recombinant MVAs in a combination treatment may be administered together or may be administered to the subject at separate times, so long as both are present together in the subject for a period of time (such as, for example, at least several hours, at least 12 hours, at least 24 hours, or at least 2 or more days). In some embodiments, the IL-12 and 4-1BBL are encoded by the same recombinant MVA; that is, in some embodiments, a recombinant MVA of the invention comprises a nucleic acid encoding a TAA, a nucleic acid encoding IL-12, and a nucleic acid encoding 4-1BBL.

Thus, in some methods of the invention, a recombinant MVA encoding IL-12 and optionally a TAA is injected intratumorally or intravenously into a subject having tumors. In some methods of the invention, a recombinant MVA encoding 4-1BBL and optionally a TAA is injected intratumorally or intravenously into a subject in combination with a recombinant MVA encoding IL-12 and optionally the same TAA or a different TAA. In embodiments where IL-12 and 4-1BBL are encoded by separate recombinant MVAs, at least one of the recombinant MVAs encodes at least one TAA, and in some embodiments both recombinant MVAs encode a TAA. In some embodiments, a recombinant MVA encoding a TAA, IL-12, and 4-1BBL is injected into a subject to provide the combination of MVA-encoded IL-12 and 4-1BBL. That is, in some embodiments, the IL-12, 4-1BBL, and TAA are all encoded by the same recombinant MVA, which can be administered to a subject to stimulate an immune response. In some embodiments, the recombinant MVA encoding a TAA, 4-1BBL, and IL-12 is injected intratumorally at least once, or at least two times or at least three times. In such embodiments when the recombinant MVA encoding a TAA, 4-1BBL, and IL-12 is injected intratumorally more than once, the injections can occur within several days of each other or within several weeks of each other, for example, at least three days apart or at least four days or a week apart, or within a month of each other or within two months of each other.

The invention also provides recombinant MVAs for preparing a medicament for intratumoral or intravenous injection for the treatment of tumors and/or to increase an immune response in a subject to a tumor. In some embodiments, this medicament comprises a recombinant MVA encoding a TAA and IL-12; in some embodiments, the medicament further comprises a recombinant MVA encoding 4-1BBL and optionally the same TAA or a different TAA. In some embodiments, the medicament comprises a recombinant MVA encoding at least one TAA, IL-12, and 4-1BBL; in these embodiments, the nucleic acids encoding each of the TAA, IL-12, and 4-1BBL may be adjacent to each other in the recombinant MVA or may be separated by nucleic acids encoding one or more other genes, or may be inserted into different locations in the recombinant MVA.

The instant inventors demonstrate in the working examples provided herein that a recombinant MVA encoding a tumor-associated antigen (TAA) and IL-12 administered intratumorally increases and enhances the immune response of a subject to the antigen. In this manner, the invention provides improved treatment of a subject having at least one tumor, including for example a human cancer patient. More particularly, the inventors demonstrated that various recombinant MVAs and combinations thereof of the present invention caused increased inflammation in the tumor when injected intratumorally. The indicia of systemic inflammation that were observed included increased serum IL-12 p70, M-CSF, and IL-33, increased antigen-specific CD8+ T cells, increased percentages of CD8+ T cells expressing IFN-gamma and TNF-alpha, decrease in tumor size and/or growth rate, improved survival, and the like.

In addition, data presented in the working examples herein showed that subjects cured of tumors following treatment with recombinant MVA encoding IL-12 or the combination of recombinant MVA encoding IL-12 and recombinant MVA encoding 4-1BBL were more likely to reject tumors when subsequently challenged with newly implanted tumors. In this manner, the invention provides compositions and methods of treatment that reduce the likelihood of recurrence of tumors.

In other embodiments, the inventors demonstrated that intraperitoneal injection of recombinant MVA encoding IL-12 enhanced anti-tumor efficacy of the treatment against intraperitoneal tumors in comparison to intravenous administration of the recombinant MVA. In this manner, the invention provides methods of treating a subject having intraperitoneal tumors comprising intraperitoneal injection of recombinant MVA encoding IL-12. In this manner, the invention also provides a medicament for intraperitoneal injection to treat an intraperitoneal tumor comprising a recombinant MVA encoding IL-12. In some embodiments, this recombinant MVA also encodes a TAA.

Accordingly, in one embodiment, the present invention includes a method for enhancing the immune response, reducing tumor size, and/or increasing survival in a subject having a cancerous tumor, the method comprising intratumorally administering to the subject a recombinant modified Vaccinia Ankara (MVA) comprising a first nucleic acid encoding a tumor-associated antigen (TAA) and a second nucleic acid encoding IL-12, wherein the intratumoral administration of the recombinant MVA increases and/or enhances an inflammatory response in the tumor, decreases the size of the tumor, and/or decreases the growth rate of the tumor, and/or increases overall survival of the subject as compared to the result expected from injection of MVA alone. In some embodiments, this method further comprises intratumorally administering to the subject a recombinant MVA comprising a nucleic acid encoding 4-1BBL and optionally also comprising a nucleic acid encoding a TAA that is the same or different from the TAA encoded by another recombinant MVA administered to said subject. In embodiments utilizing more than one recombinant MVA in combination, the TAA may be encoded by either the recombinant MVA that also encodes IL-12 or the recombinant MVA that also encodes 4-1BBL.

In some embodiments, the present invention includes a method for increasing and/or enhancing the immune response, reducing tumor size, and/or increasing survival in a subject having a tumor, the method comprising intratumorally administering to the subject a recombinant modified Vaccinia Ankara (MVA) comprising a first nucleic acid encoding a tumor-associated antigen (TAA) and a second nucleic acid encoding IL-12, wherein the intratumoral administration of the recombinant MVA increases and/or enhances an inflammatory response in the tumor, decreases the size of the tumor, decreases the growth rate of the tumor, and/or increases overall survival of the subject as compared to a non-intratumoral injection of a recombinant MVA virus comprising a first and second nucleic acid encoding a TAA and IL-12, or as compared to an intratumoral or non-intratumoral injection of MVA alone.

In an additional embodiment, the present invention includes a method for enhancing the immune response, reducing tumor size, and/or increasing survival in a subject having a cancerous tumor, the method comprising intratumorally and/or intravenously administering to the subject a recombinant modified Vaccinia Ankara (MVA) comprising a first nucleic acid encoding a tumor-associated antigen (TAA), a second nucleic acid encoding IL-12, and a third nucleic acid encoding 4-1BBL (CD137L) wherein the administration of the recombinant MVA enhances an inflammatory response in the cancerous tumor, increases tumor reduction, and/or increases overall survival of the subject as compared to an injection of MVA alone or injection of a recombinant MVA comprising a first and second nucleic acid encoding a TAA, IL-12, and a 4-1BBL antigen administered by a different route of injection (i.e., non-intratumoral or non-intravenous injection).

In an additional embodiment, the present invention includes a method for enhancing the immune response, reducing tumor size, and/or increasing survival in a subject having at least one peritoneal tumor, the method comprising intraperitoneally administering to the subject a recombinant modified Vaccinia Ankara (MVA) comprising a first nucleic acid encoding a tumor-associated antigen (TAA) and a second nucleic acid encoding IL-12, wherein the intraperitoneal administration of the recombinant MVA enhances T-cell responses specific to the TAA as compared to injection of MVA alone or to a non-intraperitoneal injection of a recombinant MVA comprising a nucleic acid encoding a TAA and a nucleic acid encoding IL-12. In another embodiment, the present invention includes a method for enhancing the immune response, reducing tumor size, and/or increasing survival in a subject having a cancerous tumor, the method comprising intraperitoneally administering to the subject a recombinant modified Vaccinia Ankara (MVA) comprising a nucleic acid encoding IL-12, wherein the intraperitoneal administration of the recombinant MVA increases and/or enhances an inflammatory response in the tumor, decreases the size of the tumor, and/or decreases the growth rate of the tumor, and/or increases overall survival of the subject as compared to the result expected from injection of MVA alone.

In some embodiments, the invention includes a method for enhancing the immune response, reducing tumor size, and/or increasing survival in a subject having a tumor, the method comprising intratumorally administering to the subject a recombinant modified Vaccinia Ankara (MVA) comprising a first nucleic acid encoding a tumor-associated antigen (TAA) and a second nucleic acid encoding IL-12 and a second recombinant MVA comprising a nucleic acid encoding 4-1BBL, wherein the administration of the recombinant MVAs enhances T cell responses specific to the TAA as compared to intratumoral injection of MVA alone or as compared to a non-intratumoral injection of a recombinant MVA virus comprising a first and second nucleic acid encoding a TAA and IL-12. In some embodiments, the TAA is encoded by a second recombinant MVA that also encodes 4-1BBL rather than by the recombinant MVA that encodes IL-12.

In some embodiments, the invention includes a method for reducing tumor size, and/or increasing survival in a subject having more than one tumor, the method comprising intratumorally administering to a particular tumor in the subject a recombinant modified Vaccinia Ankara (MVA) comprising a first nucleic acid encoding a tumor-associated antigen (TAA), a second nucleic acid encoding IL-12, and a third nucleic acid encoding 4-1BBL, whereby the administration of the recombinant MVA to said tumor decreases the growth rate and/or size of another tumor in the subject that was not injected intratumorally with said recombinant MVA(s). In this manner, the invention provides a method of stimulating an immune response against a tumor and/or decreasing the size or growth rate of a tumor comprising intratumoral injection of a different tumor. In some embodiments of this method, the subject is injected intratumorally with the recombinant MVA more than one time, or at least two times or at least three times. In such embodiments, if the subject is injected with the recombinant MVA more than one time, a second or third injection can be administered within four days or a week of the first injection, or may be administered at least a week or at least 2 or 3 weeks or at least a month after the first injection.

In yet another embodiment, the present invention includes a method of inducing an enhanced inflammatory response in a cancerous tumor of a subject and/or systemically in the subject, the method comprising intratumorally administering to the subject a recombinant modified Vaccinia Ankara (MVA) comprising a first nucleic acid encoding a first heterologous tumor-associated antigen (TAA) and a second nucleic acid encoding IL-12, wherein the intratumoral administration of the recombinant MVA generates an enhanced inflammatory response in the tumor as compared to an inflammatory response generated by or expected to be generated by injection with MVA alone or by a non-intratumoral injection of a recombinant MVA virus comprising a first and second nucleic acid encoding a heterologous tumor-associated antigen and IL-12.

In yet another embodiment, the present invention includes a method of inducing an enhanced inflammatory response in a cancerous tumor of a subject, the method comprising intratumorally administering to the subject a recombinant modified Vaccinia Ankara (MVA) encoding IL-12 and a second recombinant MVA encoding 4-1BBL, wherein either or both of said recombinant MVAs further encodes a heterologous TAA, wherein the intratumoral administration of the recombinant MVA generates an enhanced inflammatory response in the tumor as compared to an inflammatory response generated by an injection of MVA alone (i.e., an MVA not encoding heterologous antigens or genes) or a non-intratumoral injection of a recombinant MVA virus comprising a first and second nucleic acid encoding a heterologous tumor-associated antigen and IL-12.

In yet another embodiment, the present invention includes a method of inducing an increased and/or enhanced inflammatory response in a cancerous tumor of a subject, the method comprising intratumorally administering to the subject a recombinant modified Vaccinia Ankara (MVA) comprising a first nucleic acid encoding a first heterologous tumor-associated antigen (TAA), a second nucleic acid encoding IL-12, and a third nucleic acid encoding a 4-1BBL antigen, wherein the administration of the recombinant MVA generates an enhanced inflammatory response in the tumor as compared to an inflammatory response generated by an intratumoral or non-intratumoral injection of MVA alone or a non-intratumoral injection of a recombinant MVA comprising a first nucleic acid encoding a heterologous tumor-associated antigen, a second nucleic acid encoding IL-12, and a third nucleic acid encoding a 4-1BBL antigen.

In various additional embodiments, the present invention provides a recombinant modified Vaccinia Ankara (MVA) for use in preparing a medicament to treat cancer or to enhance the immune response in a subject to a cancerous tumor, the recombinant MVA comprising (a) a first nucleic acid encoding a tumor-associated antigen (TAA) and (b) a second nucleic acid encoding IL-12. Optionally, the recombinant MVA further comprises a third nucleic acid encoding 4-1BBL. Alternatively, the recombinant MVA is provided in combination with a second recombinant MVA comprising a nucleic acid encoding 4-1BBL and optionally a TAA that is the same or is different from the TAA encoded by the first nucleic acid.

In various additional embodiments, the present invention includes a recombinant modified Vaccinia Ankara (MVA) for use in enhancing the immune response of a subject to a tumor, the recombinant MVA comprising (a) a first nucleic acid encoding a tumor-associated antigen (TAA) and (b) a second nucleic acid encoding 4-1BBL. Optionally, the recombinant MVA is provided in a combination further comprising a second recombinant MVA comprising a third nucleic acid encoding IL-12.

In various additional embodiments, the present invention includes a recombinant modified Vaccinia Ankara (MVA) for use in preparing a medicament to treat cancer or enhance the immune response in a subject having cancer, the recombinant MVA comprising: (a) a first nucleic acid encoding a tumor-associated antigen (TAA); (b) a second nucleic acid encoding IL-12; and (c) a third nucleic acid encoding 4-1BBL.

In embodiments where more than one recombinant MVA is administered in a combination treatment, the recombinant MVAs can be administered at the same time or at different times so long as they are present in the subject together for some period of time. In these embodiments, unless otherwise specified, the recombinant MVAs can be administered by the same route(s) and/or location of administration or by a different location and/or route or routes of administration. That is, in some embodiments, a first recombinant MVA is administered intratumorally to a particular tumor in the subject and a second or subsequent recombinant MVA is administered intratumorally to a different tumor in the subject, or is administered intravenously, subcutaneously, intraperitoneally, or by some other route of administration. In some embodiments, a first recombinant MVA is administered intraperitoneally to a subject and a second or subsequent recombinant MVA is administered by a different route of administration, e.g., is administered intravenously, subcutaneously, intratumorally, or by some other route of administration

In some embodiments, the TAA encoded by at least one recombinant MVA is selected from the group consisting of: carcinoembryonic antigen (CEA), mucin 1 cell surface associated (MUC-1), prostatic acid phosphatase (PAP), prostate specific antigen (PSA), human epidermal growth factor receptor 2 (HER-2), survivin, tyrosine related protein 1 (TRP1), tyrosine related protein 2 (TRP2), Brachyury, Preferentially Expressed Antigen in Melanoma (PRAME), Folate receptor 1 (FOLR1), Human endogenous retrovirus-K envelope (HERV-K-env), Human endogenous retrovirus-K-gag (HERV-K-gag), and combinations thereof. In some embodiments, the TAA encoded by a recombinant MVA is expressed by at least one tumor in the subject to be treated, or is likely or is suspected to be expressed by at least one tumor in the subject to be treated.

The compositions and methods of the present invention enhance multiple aspects of a subject's immune response. In this manner, the invention provides improved treatment of a subject having at least one tumor, including for example a cancer patient. More particularly, the inventors demonstrated that various embodiments of the present invention injected intratumorally caused increased inflammatory responses in the tumor and that may also be detectable in the blood serum of the subject. These indicia of systemic inflammation can include increased production of IL-12 p70, M-CSF, and IL-33; increased antigen-specific CD8+ T cells, increased percentages of CD8+ T cells expressing IFN-gamma and TNF-alpha, decrease in tumor size and/or growth rate, improved survival of treated subjects, and the like, and can be detected by assays known in the art by evaluating the tumor and/or the peripheral blood serum, assessing survival at regular intervals, and the like.

Intratumoral administration of recombinant MVA encoding IL-12 generates an enhanced antitumor effect. In at least one aspect, the present invention includes a recombinant MVA encoding a TAA and IL-12 (rMVA-TAA-IL-12) that is administered intratumorally, wherein the intratumoral administration enhances an anti-tumor effect, as compared to an intratumoral administration of a recombinant MVA without IL-12, or as compared to a non-intratumoral administration of a recombinant MVA encoding IL-12 (for example, such as a subcutaneous administration of a recombinant MVA encoding IL-12). These enhanced antitumor effects include, for example: an increase in immune response to the tumor and/or a tumor antigen expressed by the tumor; a decrease in tumor size and/or growth rate of injected tumors as well as other, non-injected tumors in a subject; and also include an increase in survival of treated subjects.

In other embodiments, intraperitoneal administration of recombinant MVA encoding IL-12 generates an enhanced antitumor effect against intraperitoneal tumors. In this manner, the present invention includes a recombinant MVA encoding a TAA and IL-12 (rMVA-TAA-IL-12) that is administered intraperitoneally to a subject with intraperitoneal tumors, wherein the intraperitoneal administration enhances an anti-tumor effect, as compared to an intraperitoneal administration of a recombinant MVA without IL-12, or as compared to a non-intraperitoneal administration of a recombinant MVA encoding IL-12 (for example, such as a subcutaneous administration of a recombinant MVA encoding IL-12). These enhanced antitumor effects include decrease in tumor size and/or growth rate of tumors, and also include an increase in survival of treated subjects. In some embodiments, the recombinant MVA encodes IL-12 (“MVA-IL-12”) and is administered intraperitoneally to a subject with intraperitoneal tumors. The recombinant MVA encoding IL-12 tends to localize to the omentum and in this manner the invention provides a method of increasing the amount of IL-12 in the omentum, comprising administering a recombinant MVA encoding IL-12 to a subject.

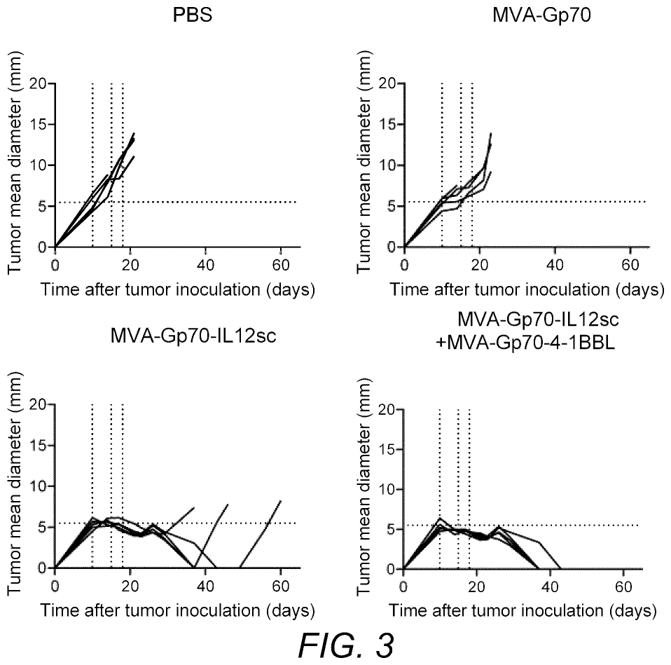

As part of the present disclosure, a recombinant MVA comprising one or more nucleic acids encoding a TAA and IL-12 was administered intratumorally to a subject. Shown in FIG. 3, an intratumoral injection of MVA-gp70-IL-12sc resulted in a significant decrease in tumor volume as compared to recombinant MVA-gp70. In addition, a recombinant MVA comprising one or more nucleic acids encoding a TAA and IL-12 was administered intratumorally to a subject in combination with a recombinant MVA comprising one or more nucleic acids encoding a TAA and 4-1BBL. Shown in FIG. 3, an intratumoral injection of MVA-gp70-IL-12sc and MVA-gp70-4-1BBL resulted in the tumor in each treated individual shrinking to an undetectable size. Thus, administration of the combination of MVA encoding IL-12 and MVA encoding 4-1BBL of the present invention advantageously provides a more effective anti-tumor treatment.

### Definitions

As used herein, the singular forms “a,” “an,” and “the” include plural references unless the context clearly indicates otherwise. Thus, for example, reference to “a nucleic acid” includes one or more of the nucleic acid and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. Those skilled in the art will recognize, or be able to ascertain using no more than routine experimentation, many equivalents to the specific embodiments of the invention described herein. Such equivalents are intended to be encompassed by the present invention.

Throughout this specification and the claims which follow, unless the context requires otherwise, the word “comprise,” and variations such as “comprises” and “comprising,” will be understood to imply the inclusion of a stated integer or step or group of integers or steps but not the exclusion of any other integer or step or group of integer or step. When used herein, the term “comprising” can be substituted with the term “containing” or “including” or sometimes with the term “having.” Any of the aforementioned terms (comprising, containing, including, having), though less preferred, whenever used herein in the context of an aspect or embodiment of the present invention can be substituted with the term “consisting of.” When used herein, “consisting of” excludes any element, step, or ingredient not specified in the claim element. When used herein, “consisting essentially of” does not exclude materials or steps that do not materially affect the basic and novel characteristics of the claim.

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

“Mutated” or “modified” protein or antigen as described herein is as defined herein any modification to a nucleic acid or amino acid, such as deletions, additions, insertions, and/or substitutions.

“Percent (%) sequence homology or identity” with respect to nucleic acid sequences described herein is defined as the percentage of nucleotides in a candidate sequence that are identical with the nucleotides in the reference sequence (i.e., the nucleic acid sequence from which it is derived), after aligning the sequences and introducing gaps, if necessary, to achieve the maximum percent sequence identity, and not considering any conservative substitutions as part of the sequence identity. Alignment for purposes of determining percent nucleotide sequence identity or homology can be achieved in various ways that are within the skill in the art, for example, using publicly available computer software such as BLAST, ALIGN, or Megalign (DNASTAR) software. Those skilled in the art can determine appropriate parameters for measuring alignment, including any algorithms needed to achieve maximum alignment over the full length of the sequences being compared.

For example, an appropriate alignment for nucleic acid sequences is provided by the local homology algorithm of Smith and Waterman ((1981) Advances in Applied Mathematics 2: 482-489). This algorithm can be applied to amino acid sequences by using the scoring matrix developed by Dayhoff, Atlas of Protein Sequences and Structure, M. O. Dayhoff ed., 5 suppl. 3: 353-358, National Biomedical Research Foundation, Washington, D.C., USA, and normalized by Gribskov ((1986) Nucl. Acids Res. 14(6): 6745-6763). An exemplary implementation of this algorithm to determine percent identity of a sequence is provided by the Genetics Computer Group (Madison, Wisconsin, USA) in the “BestFit” utility application. The default parameters for this method are described in the Wisconsin Sequence Analysis Package Program Manual, Version 8 (1995) (available from Genetics Computer Group, Madison, Wisconsin, USA). A preferred method of establishing percent identity in the context of the present invention is to use the MPSRCH package of programs copyrighted by the University of Edinburgh, developed by Collins and Sturrok, and distributed by IntelliGenetics, Inc. (Mountain View, California, USA). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: blast.ncbi.nlm.nih.gov/.

The term “prime-boost vaccination” or “prime-boost regimen” refers to a vaccination strategy or regimen using a first priming injection of a vaccine targeting a specific antigen followed at intervals by one or more boosting injections of the same vaccine. Prime-boost vaccination may be homologous or heterologous. A homologous prime-boost vaccination uses a vaccine comprising the same antigen and vector for both the priming injection and the one or more boosting injections. A heterologous prime-boost vaccination uses a vaccine comprising one antigen and/or vector for the priming injection and a different antigen and/or vector for the one or more boosting injections. For example, a homologous prime-boost vaccination uses a recombinant poxvirus comprising nucleic acids expressing one or more antigens for the priming injection and the same recombinant poxvirus expressing one or more antigens for the one or more boosting injections. In contrast, a heterologous prime-boost vaccination uses a recombinant poxvirus comprising nucleic acids expressing one or more antigens for the priming injection and a different recombinant poxvirus expressing one or more different antigens and/or comprising a different vector for the one or more boosting injections.

The term “recombinant” means a polynucleotide, virus, or vector of semisynthetic or synthetic origin which either does not occur in nature or is linked to another polynucleotide in an arrangement not found in nature. By “recombinant MVA” or “rMVA” as used herein is generally intended a modified vaccinia Ankara (MVA) that comprises at least one polynucleotide encoding a heterologous gene, such as, for example, a tumor associated antigen (TAA). In some embodiments, the recombinant MVA is MVA-BN or a derivative thereof.

The term “subject” means an animal having or suspected of having at least one tumor; thus, in some embodiments, a “subject” is a human cancer patient. A subject can be any animal, particularly a mammal, such as, for example, a cat, a dog, a horse, a cow, a sheep, or any other animal expected to benefit from or treated with the compositions and/or methods of the invention.

As used herein, a “transgene” or “heterologous” gene is understood to be a nucleic acid or amino acid sequence which is not present in the wild-type poxviral genome (e.g., the genome of Vaccinia, Fowlpox, or MVA). The skilled person understands that a “transgene” or “heterologous gene,” when present in a poxvirus such as Vaccinia virus, is to be incorporated into the poxviral genome in such a way that, following administration of the recombinant poxvirus to a host cell, it is expressed as the corresponding heterologous gene product, i.e., as the “heterologous antigen” and/or “heterologous protein.” Expression is normally achieved by operatively linking the heterologous gene to regulatory elements that allow expression in the poxvirus-infected cell. Preferably, the regulatory elements include a natural or synthetic poxviral promoter.

A “vector” refers to a recombinant DNA or RNA plasmid or virus that can comprise a heterologous polynucleotide. The heterologous polynucleotide may comprise a sequence of interest for purposes of prevention or therapy, and may optionally be in the form of an expression cassette. As used herein, a vector is used to transfer genetic material into a cell but is not necessarily capable of replication in the ultimate target cell or subject. The term includes cloning vectors and viral vectors.

The term “polypeptide” refers to a polymer of two or more amino acids joined to each other by peptide bonds or modified peptide bonds. The amino acids may be naturally occurring as well as non-naturally occurring, or a chemical analogue of a naturally occurring amino acid. The term also refers to proteins, i.e., functional biomolecules comprising at least one polypeptide; when comprising at least two polypeptides, these may form complexes, be covalently linked, or may be non-covalently linked. The polypeptide(s) in a protein can be glycosylated and/or lipidated and/or comprise prosthetic groups. As used herein, reducing tumor volume or size (or a reduction in tumor volume or size) can be characterized as a reduction in tumor volume and/or size but can also be characterized in terms of related clinical trial endpoints, as is understood in the art. Some exemplary clinical trial endpoints associated with a reduction in tumor volume and/or size can include, but are not limited to, Response Rate (RR), Objective response rate (ORR), and so forth.

As used herein, an increase in survival rate can be characterized as an increase in survival of a subject (e.g., a human cancer patient), but can also be characterized in terms of clinical trial endpoints understood in the art. Some exemplary clinical trial endpoints associated with an increase in survival rate include, but are not limited to, Overall Survival rate (OS), Progression Free Survival (PFS) and so forth.

### Combinations and Methods

In various embodiments, the present invention comprises a recombinant MVA comprising a first nucleic acid encoding a tumor-associated antigen (TAA) and a second nucleic acid encoding IL-12, that when administered intratumorally induces both an inflammatory response and an enhanced T cell response as compared to an inflammatory response and a T cell response induced by a non-intratumoral administration of MVA alone or non-intratumoral administration of a recombinant MVA comprising a first nucleic acid encoding a TAA and a second nucleic acid encoding IL-12.

In various additional embodiments, the present invention comprises an MVA comprising a first nucleic acid encoding a tumor-associated antigen (TAA), a second nucleic acid encoding IL-12, and a third nucleic acid encoding 4-1BBL, that when administered intratumorally induces both an enhanced intratumoral inflammatory response and an enhanced T cell response as compared to the responses expected to be induced by administration of MVA alone or a recombinant MVA encoding less than all of the TAA, IL-12, and 4-1BBL components (e.g., a recombinant MVA encoding only IL-12, or only 4-1BBL).

In other embodiments, the invention provides recombinant-MVA-encoded IL-12 and 4-1BBL that are encoded by separate recombinant MVAs, at least one of which also encodes a Tumor Associated Antigen (TAA). In these embodiments, the recombinant MVAs are administered in combination such that both recombinant MVAs are present together in the subject for a period of time, even though they may be administered to the subject by different routes of administration at different times. In some embodiments, two or more recombinant MVAs are administered to a subject intratumorally together (i.e., in the same medicament or formulation).

Increased or Enhanced Inflammation Response. In various aspects of the present disclosure it was determined that administration of a recombinant MVA of the invention induces an increased or enhanced inflammatory response, as compared to an administration of MVA alone. This increased or enhanced inflammatory response can be detected, for example, by measuring cytokine levels in the subject's blood and/or plasma, or may be detected at or near the site of administration, such as, for example, in a tumor that was injected intratumorally. Thus, in an aspect of the present invention it was determined that an intratumoral administration of a recombinant MVA of the invention induces an increased or enhanced inflammatory response in a tumor, as compared to an administration of MVA alone.

In some embodiments, a recombinant MVA encoding IL-12 is injected intraperitoneally to treat a subject and induces an increased or enhanced inflammatory response in at least one peritoneal tumor and/or in the omentum.

In some embodiments, the subject being treated with the methods of the invention has at least one tumor that is peritoneal carcinomatosis or has malignant ascites or a metastatic tumor of the omentum, preferably derived from an abdominal malignancy, more preferably derived from ovarian or colorectal cancer. In some embodiments, the subject is being treated for a tumor that is an abdominal malignancy, preferably metastasizing into the peritoneal cavity and/or the omentum. In some embodiments, the subject has a tumor that is a tumor of ovarian or colorectal cancer.

In some embodiments, treatment of a subject with a method of the invention increases the likelihood of survival of the subject. In some embodiments, treatment of a subject with a method of the invention induces an antigen-specific immune or T cell response, or IFN-γ production in the peritoneal cavity of a subject, and/or in the omentum.

In some embodiments, intraperitoneal administration is carried out in a prime-boost regimen.

In another embodiment, the invention provides a pharmaceutical preparation or composition comprising the recombinant MVA of the invention which pharmaceutical preparation or composition is adapted to intraperitoneal administration.

In yet another embodiment, the invention provides the recombinant MVA of the invention for use in increasing the overall survival of a subject, preferably a human, preferably suffering from peritoneal carcinomatosis or malignant ascites or a metastatic tumor of the omentum, preferably derived from an abdominal malignancy, more preferably derived from ovarian or colorectal cancer, wherein the recombinant MVA is administered intraperitoneally.

In yet another aspect, the invention provides the recombinant MVA of the invention for use in reducing signs and symptoms of peritoneal carcinomatosis or malignant ascites or a metastatic tumor of the omentum in a subject, preferably a human; in some embodiments, the tumor is derived from an abdominal malignancy, such as, for example, ovarian or colorectal cancer, wherein the recombinant MVA is administered intraperitoneally.

In yet another aspect, the invention provides the recombinant MVA of the invention for use in inducing an antigen-specific immune or T cell response, or IFN-γ production in the peritoneal cavity of a subject suffering from peritoneal carcinomatosis or malignant ascites or a metastatic tumor of the omentum, for example, derived from an abdominal malignancy, such as ovarian or colorectal cancer, wherein the recombinant MVA is administered intraperitoneally.

In at least one aspect, an “increased inflammatory response” or “enhanced inflammation response” according to the present disclosure is characterized by one or more of the following: increased production of IL-12 p70, M-CSF, and/or IL-33; increased antigen-specific CD8+ T cells, increased percentages of CD8+ T cells expressing IFN-gamma and TNF-alpha, decrease in tumor size and/or growth rate, improved survival of treated subjects, and the like, which can be detected by assays known in the art. As used herein, “increased inflammatory response” generally refers to an increase in production of a particular cytokine or cell type associated with inflammation, in comparison to baseline levels prior to treatment according to methods of the invention and/or treatment with compositions of the invention. For example, in an “increased inflammatory response,” the amount of a cytokine or cell type is increased by at least 10%, 20%, 30%, 50%, 70%, or 100% or more in comparison to baseline levels prior to treatment according to methods of the invention and/or treatment with compositions of the invention.

As used herein, “enhanced inflammatory response” generally refers to an inflammatory response in which a new cytokine or new cell population is produced that was not detectable or was only detectable at trace amounts prior to treatment according to methods of the invention and/or treatment with compositions of the invention.

The compositions and methods of the present invention enhance multiple aspects of a subject's immune response. In this manner, the invention provides improved treatment of a subject having at least one tumor, including for example a cancer patient. More particularly, the inventors demonstrated that various recombinant MVAs and combinations thereof of the present invention when injected intratumorally or intraperitoneally caused increased inflammatory responses in the tumor that may be detectable in the tumor and may also be detectable in the blood serum of the subject. These indicia of systemic inflammation can include increased production of IL-12 p70, M-CSF, and IL-33; increased antigen-specific CD8+ T cells, increased percentages of CD8+ T cells expressing IFN-gamma and TNF-alpha, decrease in tumor size and/or growth rate, improved survival of treated subjects, and the like, and can be detected by assays known in the art by evaluating the tumor and/or the peripheral blood serum, assessing survival at regular intervals, and the like.

Thus, whether an inflammatory response is enhanced or increased in a tumor and/or tumor cells in accordance with present disclosure can be determined by measuring to determine whether there is an increase in expression of one or more molecules which are indicative of an increased inflammatory response, including the secretion of chemokines and cytokines as is known in the art. Exemplary inflammatory response markers include one or more of IL-12 p70, M-CSF, IL-33, IFN-gamma, and TNF-alpha. These molecules and the measurement thereof are validated assays that are understood in the art and can be carried out according to known techniques. See, e.g., Borrego et al. ((1999) Immunology 7(1): 159-165).

The increased or enhanced inflammatory response provided by the compositions and methods of the invention can also produce decreases in the volume and/or mean diameter of at least one tumor in the treated subject. In this manner, the invention provides methods of decreasing the volume, size, and/or growth rate of at least one tumor in a subject. In some embodiments, treatment with the compositions and/or methods of the invention produces a decrease in the volume, size, and/or growth rate of at least one tumor of at least 10%, 20%, 30%, 50%, or more in comparison to the volume, size, and/or growth rate of said tumor prior to treatment.

Enhanced T Cell response. In accordance with the present application, an “enhanced T cell response” is characterized by one or more of the following: (1) an increase in frequency of CD8⁺ T cells; (2) an increase in CD8⁺ T cell activation; and (3) an increase in CD8⁺ T cell proliferation. Thus, whether a T cell response is enhanced in accordance with the present application can be determined by measuring the expression of one or more molecules which are indicative of: (1) an increase in CD8⁺ T cell frequency; (2) an increase in CD8⁺ T cell activation; and/or (3) an increase CD8⁺ T cell proliferation. Exemplary markers that are useful in measuring CD8⁺ T cell frequency, activation, and proliferation include IFN-γ, TNF-α, and/or CD44, as is known in the art. Measuring antigen specific T cell frequency can also be measured by MHC multimers such as pentamers or dextramers; such measurements and assays as well as others suitable for use in evaluating methods and compositions of the invention are validated and understood in the art.

In one aspect, an increase in CD8⁺ T cell frequency is characterized by an increase of at least 2-fold, 3-fold, 5-fold, or 10-fold or more in IFN-γ and/or dextramer+CD8⁺ T cells compared to the pre-treatment/baseline. An increase in CD8⁺ T cell activation is characterized, for example, as at least a 2-fold increase in the number of CD8+ T cells and/or at least a 2-fold increase in CD69 and/or CD44 expression compared to pre-treatment/baseline expression. An increase in CD8⁺ T cell proliferation is characterized, for example, as at least a 2-fold increase in Ki67 expression compared to pre-treatment/baseline expression.

In an alternative aspect, an increased or enhanced T cell response is characterized by an increase in CD8⁺ T cell expression of effector cytokines and/or an increase of cytotoxic effector functions. An increase in expression of effector cytokines can be measured, for example, by expression of one or more of IFN-γ, TNF-α, and/or IL-2 compared to pre-treatment/baseline. An increase in cytotoxic effector functions, for example, can be measured by expression of one or more of CD107a, granzyme B, and/or perforin and/or antigen-specific killing of target cells. The assays, cytokines, markers, and molecules described herein and the measurement thereof are validated and understood in the art and can be carried out according to known techniques. Additionally, assays for measuring cytokines and T cell responses can be found in the working examples.

In yet additional embodiments, the combinations and methods described herein are for use in treating a human cancer patient. In preferred embodiments, the cancer patient is suffering from and/or is diagnosed with a cancer selected from the group consisting of: breast cancer, lung cancer, head and neck cancer, thyroid cancer, melanoma, gastric cancer, bladder cancer, kidney cancer, liver cancer, pancreatic cancer, prostate cancer, ovarian cancer, urothelial cancer, cervical cancer, or colorectal cancer. In yet additional embodiments, the combinations and methods described herein are for use in treating a human cancer patient suffering from and/or diagnosed with a breast cancer, colorectal cancer, or melanoma, or peritoneal carcinomatosis.

Tumor-Associated Antigens for use in the compositions and methods of the invention. In certain embodiments, an immune response is produced in a subject against a cell-associated polypeptide antigen. In certain such embodiments, a cell-associated polypeptide antigen is a tumor-associated antigen (TAA). In various embodiments, the TAA is HER2, PSA, PAP, CEA, MUC-1, survivin, TRP1, TRP2, Brachyury, Preferentially Expressed Antigen in Melanoma (PRAME), Folate receptor 1 (FOLR1), Human endogenous retrovirus-K envelope (HERV-K-env), or Human endogenous retrovirus-K-gag (HERV-K-gag), alone or in any combination thereof.

In still further embodiments, the TAA may include, but is not limited to, 5 alpha reductase, alpha-fetoprotein, AM-1, APC, April, BAGE, beta-catenin, Bcl12, bcr-abl, CA-125, CASP-8/FLICE, Cathepsins, CD19, CD20, CD21, CD23, CD22, CD33 CD35, CD44, CD45, CD46, CD5, CD52, CD55, CD59, CDC27, CDK4, CEA, c-myc, Cox-2, DCC, DcR3, E6/E7, CGFR, EMBP, Dna78, farnesyl transferase, FGF8b, FGF8a, FLK-1/KDR, folic acid receptor, G250, GAGE-family, gastrin 17, gastrin-releasing hormone, GD2/GD3/GM2, GnRH, GnTV, GP1, gp100/Pmel17, gp-100-in4, gp15, gp75/TRP1, hCG, heparanase, Her2/neu, HMTV, Hsp70, hTERT, IGFR1, IL-13R, iNOS, Ki67, KIAA0205, K-ras, H-ras, N-ras, KSA, LKLR-FUT, MAGE-family, mammaglobin, MAP17, melan-A/MART-1, mesothelin, MIC A/B, MT-MMPs, mucin, NY-ESO-1, osteonectin, p15, P170/MDR1, p53, p97/melanotransferrin, PAI-1, PDGF, uPA, PRAME, probasin, progenipoietin, PSA, PSM, RAGE-1, Rb, RCAS1, SART-1, SSX-family, STAT3, STn, TAG-72, TGF-alpha, TGF-beta, Thymosin-beta-15, TNF-alpha, TRP1, TRP2, tyrosinase, VEGF, ZAG, p16INK4, and glutathione-S-transferase.

In some embodiments, the TAA is an Endogenous Retroviral Protein (ERV), or derivative thereof. Such an ERV can be an ERV from the Human HERV-K protein family and, for example, can be a HERV-K envelope (env) protein, a HERV-K group specific antigen (gag) protein, and a HERV-K “marker of melanoma risk” (mel) protein (see, e.g., Cegolon et al. (2013) BMC Cancer 13:4).

Any TAA may be used so long as it accomplishes at least one objective or desired end of the invention, such as, for example, stimulating an immune response following administration of the MVA containing it. In some embodiments, the TAA encoded by the one or more recombinant MVAs is known to be expressed by at least one tumor in the subject, for example, based on previous testing of a sample of the tumor. Exemplary sequences of TAAs, including TAAs mentioned herein, are known in the art and are suitable for use in the compositions and methods of the invention. Sequences of TAAs for use in the compositions and methods of the invention may be identical to sequences known in the art or disclosed herein, or they may share less than 100% identity, such as at least 90%, 91%, 92%, 95%, 97%, 98%, or 99% or more sequence identity to either a nucleotide or amino acid sequence known in the art or disclosed herein. Thus, a sequence of a TAA for use in a composition or method of the invention may differ from a reference sequence known in the art and/or disclosed herein by less than 20, or less than 19, 18, 15, 14, 13, 12, 11, 10, 9, 8, 7, 6, 5, 4, 3, 2, or 1 nucleotides or amino acids, so long as it accomplishes at least one objective or desired end of the invention (for example, to help stimulate an immune response when administered to a subject as a component of a recombinant MVA). One of skill in the art is familiar with techniques and assays for evaluating TAAs to ensure their suitability for use in a recombinant MVA or method of the invention.

In certain embodiments, modifications to one or more of the TAAs, such as, but not limited to, HERV-K env, HERV-K gag, HERV-K mel, CEA, MUC-1, PAP, PSA, PRAME, FOLR1, HER2, survivin, TRP1, TRP2, or Brachyury, are made such that, after administration to a subject, polyclonal antibodies are elicited that predominantly react with the one or more of the TAAs described herein. Such antibodies could attack and eliminate tumor cells as well as prevent metastatic cells from developing into metastases. The effector mechanism of this anti-tumor effect would be mediated via complement and Antibody-Dependent Cellular Cytotoxicity (“ADCC”). In addition, the induced antibodies could also inhibit cancer cell growth through inhibition of growth-factor-dependent oligo-dimerization and internalization of the receptors. In certain embodiments, such modified TAAs could induce CTL responses directed against known and/or predicted TAA epitopes displayed by the tumor cells.

In certain embodiments, a modified TAA polypeptide antigen comprises a CTL epitope of the cell-associated polypeptide antigen and a variation, wherein the variation comprises at least one CTL epitope or a foreign TH epitope. Certain such modified TAAs can include (in one non-limiting example) one or more HER2 polypeptide antigens comprising at least one CTL epitope and a variation comprising at least one CTL epitope of a foreign TH epitope; these HER2 antigens and methods of producing the same are described in U.S. Pat. No. 7,005,498 and U.S. Patent Pub. Nos. 2004/0141958 and 2006/0008465, herein incorporated by reference.

IL-12. Structurally, IL-12 is a type I cytokine, heterodimeric protein consisting of two p35 and p40 subunits that are covalently linked. The heterodimer form is also referred to as IL-12-p70 or IL-12-p35/p40. IL-12 has many effects that promote an immune response, but some clinical studies with IL-12 had unacceptable levels of adverse events (see Lasek et al. (2014) Cancer Immunol. Immunother. 63: 419-35). IL-12 has been demonstrated to induce production of IFN-gamma, to induce T_{H}1 cell differentiation, and also to increase activation and cytotoxic function of T and NK cells (see Nguyen et al. (2020) Front. Immunol. 11: 575597). A variety of modified forms of IL-12 are known in the art and are useful in embodiments of the invention so long as they retain IL-12 function, such as, for example, increasing secretion of IFN-gamma (“IFN-γ”), etc. For example, a modified form of IL-12 known in the art is “single chain Interleukin-12,” also referred to as “IL-12 sc” or “sc IL-12.” This IL-12 sc provides the advantage of automatically having the correct stoichiometry of the p35 and p40 subunits, so that there is not excess p40 subunit produced that might exert an inhibitory effect on the full length IL-12 (see, e.g., Anderson et al. (1997) Hum. Gene Ther. 8: 1125-35). A homodimer of the p40 subunits has been shown to suppress the activity of the heterodimer form and thus would not be useful in embodiments of the invention.

In some embodiments of the invention, IL-12 is encoded by a recombinant MVA along with a tumor-associated antigen (“TAA”). In some embodiments of the invention, IL-12 is encoded by a recombinant MVA along with 4-1BBL and a TAA; alternatively, in some embodiments of the invention, 4-1BBL is encoded by a recombinant MVA separately from IL-12 and used in combination with a recombinant MVA encoding IL-12, wherein at least one of such MVAs also encodes a TAA. In some embodiments, a recombinant MVA encodes IL-12 and, optionally, also encodes a TAA. In some embodiments, the IL-12 sequence is a human IL-12 sequence. In some embodiments, the IL-12 has an amino acid sequence with at least 90%, 95%, 97% 98%, or 99% identity to SEQ ID NO: 10 or 12, or has an amino acid sequence that differs from the amino acid sequence set forth in SEQ ID NO: 10 or 12 by less than 10, 9, 8, 7, 6, 5, 4, 3, 2, or 1 amino acids, or is identical to the sequence set forth in SEQ ID NO: 10 or 12. In additional embodiments, a nucleic acid encoding IL-12 comprises a nucleic acid sequence having at least 90%, 95%, 97% 98%, or 99% identity to SEQ ID NO: 9 or 11, i.e., differing from the nucleic acid sequence set forth in SEQ ID NO: 9 or 11 by less than 20, 10, 5, 4, 3, 2, or 1 nucleic acid in the sequence, or is identical to the sequence set forth in SEQ ID NO: 9 or 11. IL-12 is well studied, so it is expected that one of skill in the art would be able to introduce sequence modifications in more variable or less conserved regions to avoid affecting gene function. It is contemplated that any IL-12 sequence is suitable for use in embodiments of the invention so long as it provides at least one function of IL-12 in an assay, such as any of the assays for IL-12 used in the working examples or otherwise known in the art.

4-1BBL (also referred to herein as “41BBL” or “4-1BB ligand”). As illustrated by the present disclosure, the inclusion of 4-1BBL along with IL-12 in one or more recombinant MVAs and related methods induces increased and enhanced anti-tumor effects following intratumoral administration to a subject. Thus, in various embodiments, in addition to encoding a TAA and IL-12, a recombinant MVA encodes a 4-1BBL antigen, either in the same or in more than one recombinant MVAs. That is, in some embodiments, a separate recombinant MVA encoding a 4-1BBL antigen is administered in combination with a recombinant MVA encoding IL-12, wherein at least one of said recombinant MVAs also encodes a TAA. In such embodiments, the inclusion of 4-1BBL as part of the combination and related methods further enhances the immune response and decrease in tumor volume as well as prolonging progression-free survival and increasing survival rate.

4-1BB/4-1BBL is a member of the TNFR/TNF superfamily. 4-1BBL is a costimulatory ligand expressed in activated B cells, monocytes and DCs, and 4-1BB is constitutively expressed by natural killer (NK) and natural killer T (NKT) cells, Tregs and several innate immune cell populations, including DCs, monocytes and neutrophils. Interestingly, 4-1BB is expressed on activated, but not resting, T cells (Wang et al. (2009) Immunol. Rev. 229: 192-215). 4-1BB ligation induces proliferation and production of interferon gamma (IFN-γ) and interleukin 2 (IL-2), as well as enhances T cell survival through the upregulation of antiapoptotic molecules such as Bcl-xL (Snell et al. (2011) Immunol. Rev. 244: 197-217). 4-1BB stimulation has been shown to enhance NK cell proliferation, IFN-γ production and cytolytic activity through enhancement of Antibody-Dependent Cell Cytotoxicity (“ADCC”) (Kohrt et al. (2011) Blood 117: 2423-32).

In some embodiments of the invention, 4-1BBL is encoded by a recombinant MVA along with IL-12 and a TAA; alternatively, in some embodiments of the invention, 4-1BBL is encoded by a recombinant MVA separately from IL-12 and used in combination with a recombinant MVA encoding IL-12, wherein at least one of such MVAs also encodes a TAA. In some embodiments, the 4-1BBL sequence is a human 4-1BBL sequence. In some embodiments, the 4-1BBL has an amino acid sequence with at least 90%, 95%, 97% 98%, or 99% identity to SEQ ID NO: 14; or has an amino acid sequence that differs from the amino acid sequence set forth in SEQ ID NO: 14 by less than 10, 9, 8, 7, 6, 5, 4, 3, 2, or 1 amino acids, or is identical to the sequence set forth in SEQ ID NO: 14. In additional embodiments, a nucleic acid encoding 4-1BBL comprises a nucleic acid sequence having at least 90%, 95%, 97% 98%, or 99% identity to SEQ ID NO: 13, i.e., differing from the nucleic acid sequence set forth in SEQ ID NO: 13 by less than 20, 10, 5, 4, 3, 2, or 1 nucleic acid in the sequence, or is identical to the sequence set forth in SEQ ID NO: 13. 4-1BBL is well studied, so it is expected that one of skill in the art would be able to introduce sequence modifications in more variable or less conserved regions to avoid affecting gene function. It is contemplated that any 4-1BBL sequence is suitable for use in embodiments of the invention so long as it provides at least one function of 4-1BBL in an assay, such as any of the assays for 4-1BBL used in the working examples or otherwise known in the art.

Recombinant MVAs. In some embodiments of the present invention, IL-12, 4-1BBL, and a TAA are encoded by the same recombinant MVA, and in some embodiments, the IL-12 and 4-1BBL are encoded by different recombinant MVAs, at least one of which also encodes a TAA, and are administered to a subject in combination. As described and illustrated by the present disclosure, the intratumoral administration of the recombinant MVAs of the present disclosure induces in various aspects an enhanced immune response in cancer patients. In other embodiments, a recombinant MVA encoding a TAA and IL-12 is administered intraperitoneally to a subject having at least one intraperitoneal tumor, and in some embodiments a recombinant MVA encoding IL-12 (and not a heterologous TAA) is administered intraperitoneally to a subject having at least one intraperitoneal tumor.

Examples of MVA strains that are useful in the practice of the present invention and that have been deposited in compliance with the requirements of the Budapest Treaty are strains MVA 572, deposited at the European Collection of Animal Cell Cultures (ECACC), Vaccine Research and Production Laboratory, Public Health Laboratory Service, Centre for Applied Microbiology and Research, Porton Down, Salisbury, Wiltshire SP4 0JG, United Kingdom, with the deposition number ECACC 94012707 on Jan. 27, 1994; and MVA 575, deposited under ECACC 00120707 on Dec. 7, 2000; and MVA-BN, deposited on Aug. 30, 2000 at the European Collection of Cell Cultures (ECACC) under number V00083008, and their derivatives.

“Derivatives” of MVA-BN refer to viruses exhibiting essentially the same replication characteristics as MVA-BN, as described herein, but exhibiting differences in one or more parts of their genomes. MVA-BN, as well as derivatives thereof, are replication incompetent, meaning a failure to reproductively replicate in vivo and in vitro. More specifically, in vitro, MVA-BN or derivatives thereof have been described as being capable of reproductive replication in chicken embryo fibroblasts (CEF), but not capable of reproductive replication in the human keratinocyte cell line HaCat (Boukamp et al. (1988) J. Cell Biol. 106: 761-771), the human bone osteosarcoma cell line 143B (ECACC Deposit No. 91112502), the human embryo kidney cell line 293 (ECACC Deposit No. 85120602), and/or the human cervix adenocarcinoma cell line HeLa (ATCC Deposit No. CCL-2). Additionally, MVA-BN or derivatives thereof have a virus amplification ratio at least two-fold less, more preferably three-fold less, than MVA-575 in HeLa cells and HaCaT cell lines. Tests and assay for these properties of MVA-BN and derivatives thereof are described in WO 02/42480 (U.S. Pub. No. 2003/0206926) and WO 03/048184 (U.S. Pub. No. 2006/0159699).

The term “not capable of reproductive replication” or “no capability of reproductive replication” in human cell lines in vitro as described in the previous paragraphs is, for example, described in WO 02/42480, which also teaches how to obtain MVA having the desired properties as mentioned above. In some instances, the term applies to a virus that has a virus amplification ratio in vitro at 4 days after infection of less than 1 using the assays described in WO 02/42480 or in U.S. Pat. No. 6,761,893, herein incorporated by reference. The term “failure to reproductively replicate” refers to a virus that has a virus amplification ratio in human cell lines in vitro as described in the previous paragraphs at 4 days after infection of less than 1. Assays described in WO 02/42480 or in U.S. Pat. No. 6,761,893 are applicable for the determination of the virus amplification ratio.

The amplification or replication of a virus in human cell lines in vitro as described in the previous paragraphs is normally expressed as the ratio of virus produced from an infected cell (output) to the amount originally used to infect the cell in the first place (input) referred to as the “amplification ratio.” An amplification ratio of “1” defines an amplification status where the amount of virus produced from the infected cells is the same as the amount initially used to infect the cells, meaning that the infected cells are permissive for virus infection and reproduction. In contrast, an amplification ratio of less than 1, i.e., a decrease in output compared to the input level, indicates a lack of reproductive replication and therefore attenuation of the virus.

By “adjuvantation” herein is intended that a particular encoded protein or component of a recombinant MVA increases the immune response produced by the other encoded protein(s) or component(s) of the recombinant MVA.

In some embodiments, the compositions, methods, and combinations of the invention increase overall survival of a treated subject. By “increase overall survival” as used herein is intended that there is a statistically significant improvement in the survival rate of treated subjects as compared to untreated subjects.

Expression Cassettes/Control Sequences. In various aspects, the one or more nucleic acids described herein are embodied in in one or more expression cassettes in which the one or more nucleic acids are operably linked to expression control sequences. “Operatively linked” or “operably linked” means that the components described are in relationship permitting them to function in their intended manner e.g., a promoter to transcribe the nucleic acid to be expressed. An expression control sequence operatively linked to a coding sequence is joined such that expression of the coding sequence is achieved under conditions compatible with the expression control sequences. The expression control sequences include, but are not limited to, appropriate promoters, enhancers, transcription terminators, a start codon at the beginning of a protein-encoding open reading frame, splicing signals for introns, and in-frame stop codons. Suitable promoters include, but are not limited to, the SV40 early promoter, an RSV promoter, the retrovirus LTR, the adenovirus major late promoter, the human CMV immediate early I promoter, and various poxvirus promoters, including but not limited to the following vaccinia virus or MVA-derived and FPV-derived promoters: the 30K promoter, the 13 promoter, the PrS promoter, the PrS5E promoter, the Pr7.5K, the PrHyb promoter, the Pr13.5 long promoter, the 40K promoter, the MVA-40K promoter, the FPV 40K promoter, 30 k promoter, the PrSynIIm promoter, the PrLE1 promoter, and the PR1238 promoter. Additional promoters are further described in WO 2010/060632, WO 2010/102822, WO 2013/189611,WO 2014/063832, and WO 2017/021776, which are incorporated fully by reference herein.

Additional expression control sequences include, but are not limited to, leader sequences, termination codons, polyadenylation signals, and any other sequences necessary for the appropriate transcription and subsequent translation of the nucleic acid sequence encoding the desired heterologous protein (e.g., a TAA, IL-12, and/or 4-1BBL) in the desired host system. The poxvirus vector may also contain additional elements necessary for the transfer and subsequent replication of the expression vector containing the nucleic acid sequence in the desired host system. It will further be understood by one skilled in the art that such vectors are easily constructed using conventional methods (Ausubel et al., (1987) in “Current Protocols in Molecular Biology,” John Wiley and Sons, New York, N.Y.) and are commercially available.

Methods and dosing regimens for administering the combination. In one or more aspects, the combinations of the present invention can be administered as part of a homologous and/or heterologous prime-boost regimen. As shown by the working examples, a homologous prime boost regimen increases a subject's specific T cell responses. Thus, in one or more embodiments there is a combination and/or method for stimulating the immune response, reducing tumor size and/or increasing survival in a subject comprising administering to the subject a combination of the instant invention, wherein the combination is administered as part of a homologous or heterologous prime-boost regimen.

### Generation of Recombinant MVA Viruses Comprising Transgenes

The recombinant MVA viruses provided herein can be generated by routine methods known in the art. Methods to obtain recombinant poxviruses or to insert exogenous coding sequences into a poxviral genome are well known to the person skilled in the art. For example, methods for standard molecular biology techniques such as cloning of DNA, DNA and RNA isolation, Western blot analysis, RT-PCR and PCR amplification techniques are described in Molecular Cloning, A Laboratory Manual (2nd ed., Sambrook et al., Cold Spring Harbor Laboratory Press (1989)), and techniques for the handling and manipulation of viruses are described in Virology Methods Manual (Mahy et al. (eds.), Academic Press (1996)). Similarly, techniques and know-how for the handling, manipulation and genetic engineering of MVA are described in Molecular Virology: A Practical Approach (Davison & Elliott (eds.), The Practical Approach Series, IRL Press at Oxford University Press, Oxford, UK (1993)(see, e.g., “Chapter 9: Expression of genes by Vaccinia virus vectors”)) and Current Protocols in Molecular Biology (John Wiley & Son, Inc. (1998) (see, e.g., Chapter 16, Section IV: “Expression of proteins in mammalian cells using vaccinia viral vector”)).

For the generation of the various recombinant MVA viruses disclosed herein, different methods may be applicable. The DNA sequence to be inserted into the virus can be placed into an E. coli plasmid construct into which DNA homologous to a section of DNA of the poxvirus has been inserted. Separately, the DNA sequence to be inserted can be ligated to a promoter. The promoter-gene linkage can be positioned in the plasmid construct so that the promoter-gene linkage is flanked on both ends by DNA homologous to a DNA sequence flanking a region of poxviral DNA containing a non-essential locus. The resulting plasmid construct can be amplified by propagation within E. coli bacteria and isolated. The isolated plasmid containing the DNA gene sequence to be inserted can be transfected into a cell culture, e.g., of chicken embryo fibroblasts (CEFs), at the same time the culture is infected with MVA virus. Recombination between homologous MVA viral DNA in the plasmid and the viral genome, respectively, can generate a poxvirus modified by the presence of foreign DNA sequences.

According to a preferred embodiment, a cell of a suitable cell culture such as, e.g., CEF cells, can be infected with an MVA virus. The infected cell can be subsequently transfected with a first plasmid vector comprising a foreign or heterologous gene or genes, such as one or more of the nucleic acids provided in the present disclosure, preferably under the transcriptional control of a poxvirus expression control element. As explained above, the plasmid vector also comprises sequences capable of directing the insertion of the exogenous sequence into a selected part of the MVA viral genome. Optionally, the plasmid vector also contains a cassette comprising a marker and/or selection gene operably linked to a poxviral promoter. Suitable marker or selection genes are, e.g., the genes encoding the green fluorescent protein, β-galactosidase, neomycin-phosphoribosyltransferase, or other markers. The use of selection or marker cassettes simplifies the identification and isolation of the generated recombinant poxvirus. However, a recombinant poxvirus can also be identified by PCR technology. Subsequently, a further cell can be infected with the recombinant poxvirus obtained as described above and transfected with a second vector comprising a second foreign or heterologous gene or genes. If this gene should be introduced into a different insertion site of the poxviral genome, the second vector will also differ in the poxvirus-homologous sequences directing the integration of the second foreign gene or genes into the genome of the poxvirus. After homologous recombination has occurred, the recombinant virus comprising two or more foreign or heterologous genes can be isolated. For introducing additional foreign genes into the recombinant virus, the steps of infection and transfection can be repeated by using the recombinant virus isolated in previous steps for infection and by using a further vector comprising a further foreign gene or genes for transfection.

Alternatively, the steps of infection and transfection as described above are interchangeable, i.e., a suitable cell can at first be transfected by the plasmid vector comprising the foreign gene and then infected with the poxvirus. As a further alternative, it is also possible to introduce each foreign gene into different viruses, co-infect a cell with all the obtained recombinant viruses, and screen for a recombinant including all the desired foreign genes. A third alternative is ligation of the DNA genome and foreign sequences in vitro followed by reconstitution of the recombined vaccinia virus DNA genome using a helper virus. A fourth alternative is homologous recombination in E. coli or other host cell between a MVA virus genome cloned as a bacterial artificial chromosome (BAC) and a linear foreign sequence flanked with DNA sequences homologous to sequences flanking the desired site of integration in the MVA virus genome.

The one or more nucleic acids of the present disclosure may be inserted into any suitable part of the MVA virus or MVA viral vector to produce a recombinant MVA of the invention. Suitable parts of the MVA virus are non-essential parts of the MVA genome. Non-essential parts of the MVA genome may be intergenic regions or known deletion sites in the MVA genome. Alternatively, or additionally, non-essential parts of the recombinant MVA can be a coding region of the MVA genome which is non-essential for viral growth. Insertion sites are not restricted to these preferred insertion sites in the MVA genome, since it is within the scope of the present invention that the nucleic acids of the present invention (e.g., encoding a TAA, IL-12, and/or 4-1BBL) and any accompanying promoters as described herein may be inserted anywhere in the viral genome as long as it is possible to obtain recombinants that can be amplified and propagated in at least one cell culture system, such as Chicken Embryo Fibroblasts (CEF cells).

Preferably, the nucleic acids of the present invention may be inserted into one or more intergenic regions (IGR) of the MVA virus. The term “intergenic region” refers to those parts of the viral genome located between two adjacent open reading frames (ORF) of the MVA virus genome, preferably between two essential ORFs of the MVA virus genome. For recombinant MVAs of the invention, in certain embodiments, the IGR is selected from IGR 07/08, IGR 44/45, IGR 64/65, IGR 88/89, IGR 136/137, and IGR 148/149. For recombinant MVAs, the nucleotide sequences may, additionally or alternatively, be inserted into one or more of the known deletion sites, i.e., deletion sites I, II, III, IV, V, or VI of the MVA genome. The term “known deletion site” refers to those parts of the MVA genome that were deleted through continuous passaging on CEF cells characterized at passage 516 with respect to the genome of the parental virus from which the MVA is derived from, in particular the parental chorioallantois vaccinia virus Ankara (CVA), e.g., as described in Meisinger-Henschel et al. ((2007) J. Gen. Virol. 88: 3249-3259).

### Vaccines

In certain embodiments, the recombinant MVA of the present disclosure can be formulated as part of a vaccine, or used to prepare a medicament that is a vaccine. For the preparation of vaccines, the MVA virus can be converted into a physiologically acceptable form.

An exemplary preparation follows. Purified virus is stored at −80° C. with a titer of 5×10⁸ TCID₅₀/ml formulated in 10 mM Tris, 140 mM NaCl, pH 7.4. For the preparation of vaccine doses, e.g., 1×10⁸-1×10⁹ particles of the virus can be lyophilized in phosphate-buffered saline (PBS) in the presence of 2% peptone and 1% human albumin in an ampoule, preferably a glass ampoule. Alternatively, the vaccine doses or shots can be prepared by stepwise freeze-drying of the virus in a formulation. In certain embodiments, the formulation contains additional additives such as, for example, mannitol, dextran, sugar, glycine, lactose, polyvinylpyrrolidone, and optionally other additives, such as antioxidants or inert gas, stabilizers, or recombinant proteins (e.g. human serum albumin) suitable for in vivo administration. The ampoule is then sealed and can be stored at a suitable temperature, for example, between 4° C. and room temperature for several months. However, for long-term storage, the ampoule is stored preferably at temperatures below −20° C., most preferably at about −80° C.

In various embodiments involving vaccination or therapy, the lyophilisate is dissolved in 0.1 to 0.5 ml of an aqueous solution, preferably physiological saline or Tris buffer such as 10 mM Tris, 140 mM NaCl pH 7.7. It is contemplated that the recombinant MVA vaccine or pharmaceutical composition of the present disclosure can be formulated in solution in a concentration range of 10⁴ to 10¹⁰ TCID₅₀/ml, 10⁵ to 5×10⁹ TCID₅₀/ml, 10⁶ to 5×10⁹ TCID₅₀/ml, or 10⁷ to 5×10⁹ TCID₅₀/ml. A preferred dose for humans comprises between 10⁶ to 10¹⁰ TCID₅₀, including a dose of 10⁶ TCID₅₀, 10⁷ TCID₅₀, 10⁸ TCID₅₀, 5×10⁸ TCID₅₀, 10⁹ TCID₅₀, 5×10⁹ TCID₅₀, or 10¹⁰ TCID₅₀. Optimization of dose and number of administrations is within the ability and knowledge of one skilled in the art.

In one or more preferred embodiments, as set forth herein, the recombinant MVA or MVAs are administered to a cancer patient intratumorally. In other embodiments, the recombinant MVA or MVAs are administered to a cancer patient intraperitoneally. In other embodiments, the recombinant MVA or MVAs are administered to a cancer patient either intratumorally, intravenously, subcutaneously, and/or intraperitoneally at the same time or at different times.

Kits, Compositions, and Methods of Use. In various embodiments, the invention encompasses kits, pharmaceutical combinations, pharmaceutical compositions, and/or immunogenic combinations comprising one or more recombinant MVAs that include the nucleic acids described herein.

It is contemplated that a kit and/or composition of the invention can comprise one or multiple containers or vials of one or more recombinant poxviruses of the present disclosure together with instructions for the administration of the recombinant MVA or MVAs. It is contemplated that in a more particular embodiment, the kit can include instructions for administering the recombinant MVA(s) in a first priming administration and then administering one or more subsequent boosting administrations of the recombinant MVA(s) in a homologous or heterologous prime-boost regimen, as appropriate.

The kits and/or compositions provided herein may generally include one or more pharmaceutically acceptable and/or approved carriers, additives, antibiotics, preservatives, diluents and/or stabilizers. Such auxiliary substances can include water, saline solution, glycerol, ethanol, wetting or emulsifying agents, pH buffering substances, and the like. Suitable carriers are typically large, slowly-metabolized molecules such as proteins, polysaccharides, polylactic acids, polyglycolic acids, polymeric amino acids, amino acid copolymers, lipid aggregates, and the like.

Certain Exemplary Embodiments. Embodiment 1 is a recombinant modified Vaccinia Ankara (MVA) for use in stimulating an immune response to a Tumor Associated Antigen (TAA) in a subject, comprising: (a) a first nucleic acid encoding a tumor-associated antigen (TAA); and (b) a second nucleic acid encoding IL-12; wherein the intratumoral administration of the recombinant MVA increases and/or enhances an inflammatory response in a tumor, reduces the growth rate and/or size of the tumor, and/or increases overall survival of the subject as compared to a non-intratumoral injection of said recombinant MVA or an injection of a recombinant MVA that does not comprise a nucleic acid encoding IL-12, wherein the MVA is administered intratumorally.

Embodiment 2 is a recombinant MVA for use according to embodiment 1, further comprising (c) a third nucleic acid encoding 4-1BBL.

Embodiment 3 is a recombinant MVA for use according to embodiment 1, wherein said TAA is an endogenous retroviral (ERV) protein.

Embodiment 4 is a recombinant MVA for use according to embodiment 1, wherein said TAA is selected from the group consisting of carcinoembryonic antigen (CEA), mucin 1 cell surface associated (MUC-1), prostatic acid phosphatase (PAP), prostate specific antigen (PSA), human epidermal growth factor receptor 2 (HER-2), survivin, tyrosine related protein 1 (TRP1), tyrosine related protein 1 (TRP2), Brachyury, FOLR1, PRAME, HERV-K-env, HERV-K-gag, and combinations thereof.

Embodiment 5 is a recombinant MVA for use in the treatment of tumors, comprising: (a) a first nucleic acid encoding a tumor-associated antigen (TAA); and (b) a second nucleic acid encoding IL-12; wherein the intratumoral administration of the recombinant MVA enhances an inflammatory response in a tumor, reduces the growth rate and/or size of the tumor, and/or increases overall survival of the subject as compared to a non-intratumoral injection of said recombinant MVA or an injection of a recombinant MVA that does not comprise a nucleic acid encoding IL-12; wherein the TAA is an endogenous retroviral (ERV) protein; and wherein the MVA is administered intratumorally.

Embodiment 6 is a recombinant MVA for use according to embodiment 5, wherein the TAA is selected from the group consisting of carcinoembryonic antigen (CEA), mucin 1 cell surface associated (MUC-1), prostatic acid phosphatase (PAP), prostate specific antigen (PSA), human epidermal growth factor receptor 2 (HER-2), survivin, tyrosine related protein 1 (TRP1), tyrosine related protein 2 (TRP2), Brachyury, FOLR1, PRAME, HERV-K-env, HERV-K-gag, and combinations thereof.

Embodiment 7 is a pharmaceutical combination comprising: (i) a recombinant modified Vaccinia Ankara (MVA), comprising: (a) a first nucleic acid encoding a tumor-associated antigen (TAA); and (b) a second nucleic acid encoding IL-12; wherein the intratumoral administration of the recombinant MVA enhances an inflammatory response in a tumor, reduces the growth rate and/or size of the tumor, and/or increases overall survival of the subject as compared to a non-intratumoral injection of said recombinant MVA or an injection of a recombinant MVA that does not comprise a nucleic acid encoding IL-12; and (ii) a pharmaceutically acceptable carrier.

Embodiment 8 is a pharmaceutical combination comprising embodiment 7 and a recombinant MVA comprising: (a) a first nucleic acid encoding a tumor-associated antigen (TAA); and (b) a second nucleic acid encoding 4-1BBL, wherein said TAA can be the same TAA as recited in embodiment 7 or can be a different TAA.

Embodiment 9 is a pharmaceutical combination according to embodiment 8, wherein said second recombinant MVA comprises a first nucleic acid encoding a TAA that is a different TAA than the one encoded by the recombinant MVA of claim 1.

Embodiment 10 is a method for reducing tumor growth and/or increasing survival in a subject having a cancerous tumor, the method comprising intratumorally administering to the subject a recombinant modified Vaccinia virus Ankara (MVA) comprising a first nucleic acid encoding a tumor-associated antigen (TAA) and a second nucleic acid encoding IL-12 and optionally a third nucleic acid encoding 4-1BBL, wherein the intratumoral administration of the recombinant MVA enhances an inflammatory response in the tumor, decreases tumor growth and/or size, and/or increases overall survival of the subject as compared to injection of a recombinant MVA that does not comprise a nucleic acid encoding IL-12 or MVA alone.

Embodiment 11 is a method according to embodiment 10, wherein the TAA is selected from the group consisting of carcinoembryonic antigen (CEA), mucin 1 cell surface associated (MUC-1), prostatic acid phosphatase (PAP), prostate specific antigen (PSA), human epidermal growth factor receptor 2 (HER-2), survivin, tyrosine related protein 1 (TRP1), tyrosine related protein 1 (TRP2), Brachyury, PRAME, FOLR1, HERV-K-env, HERV-K-gag, and combinations thereof.

Embodiment 12 is a method according to embodiment 10, wherein said MVA comprises a first nucleic acid encoding a tumor-associated antigen (TAA) and a second nucleic acid encoding IL-12, and wherein the method further comprises intratumorally administering to said subject a recombinant MVA comprising a first nucleic acid encoding a tumor-associated antigen (TAA) and a second nucleic acid encoding 4-1BBL, wherein said TAA may be the same TAA recited in claim 10 or may be a different TAA.

Embodiment 13 is a method according to embodiment 10, wherein the subject is a human cancer patient.

Embodiment 14 is a method for reducing tumor size or growth and/or increasing survival in a subject having an tumor, the method comprising intraperitoneally administering to the subject a recombinant modified Vaccinia Ankara (MVA) comprising a first nucleic acid encoding IL-12 and optionally a second nucleic acid encoding a tumor-associated antigen (TAA), wherein the administration of the recombinant MVA enhances Natural Killer (NK) cell response and enhances CD8 T cell responses specific to the TAA as compared to baseline levels prior to treatment or as compared to the expected result of injection with MVA alone.

Embodiment 15 is the method is according to embodiment 14, wherein the TAA is selected from the group consisting of carcinoembryonic antigen (CEA), mucin 1 cell surface associated (MUC-1), prostatic acid phosphatase (PAP), prostate specific antigen (PSA), human epidermal growth factor receptor 2 (HER-2), survivin, tyrosine related protein 1 (TRP1), tyrosine related protein 1 (TRP2), Brachyury, PRAME, FOLR1, HERV-K-env, HERV-K-gag, and combinations thereof.

Embodiment 16 is a method according to embodiment 14, wherein the subject is human and the tumor is intraperitoneal.

Embodiment 17 is a method according to embodiment 14, further comprising intratumorally administering to said subject a recombinant MVA comprising a first nucleic acid encoding a tumor-associated antigen (TAA) and a second nucleic acid encoding 4-1BBL, wherein said TAA may be the same TAA as embodiment 14 or may be a different TAA.

Embodiment 18 is a method of inducing an enhanced inflammatory response in a peritoneal tumor of a subject, the method comprising intraperitoneally administering to the subject a recombinant modified Vaccinia Ankara (MVA) comprising a first nucleic acid encoding IL-12 or IL-12sc and optionally a second nucleic acid encoding a heterologous tumor-associated antigen (TAA), wherein the intraperitoneal administration of the recombinant MVA generates an enhanced inflammatory response in the tumor as compared to an inflammatory response that would be generated by a non-intraperitoneal injection of a recombinant MVA virus alone.

Embodiment 19 is a method according to embodiment 18, further comprising administering to the subject a recombinant MVA comprising a first nucleic acid encoding a first heterologous tumor-associated antigen (TAA) and a second nucleic acid encoding 4-1BBL.

Embodiment 20 is a method according to embodiment 18, further comprising intraperitoneally administering to the subject a boosting dose of the same recombinant modified Vaccinia Ankara (MVA).

Embodiment 21 is a vaccine comprising any of embodiments 1-6 and a pharmaceutically acceptable carrier.

Embodiment 22 is a recombinant MVA according to any one of embodiments 1-6, a vaccine according to embodiment 21, or a pharmaceutical combination according to any one of embodiments 7-9, for use in reducing tumor size and/or increasing survival in a subject having a cancerous tumor.

Embodiment 23 is a recombinant MVA according to any one of embodiments 1-6, a vaccine according to embodiment 21, or a pharmaceutical combination according to any one of embodiments 7-9, for use in a method for reducing tumor size and/or increasing survival in a subject having a cancerous tumor, the method comprising intratumorally or intraperitoneally administering to the subject said recombinant MVA, vaccine, or pharmaceutical combination, wherein the intratumoral or intraperitoneal administration enhances an inflammatory response in the cancerous tumor, decreases tumor growth rate, increases tumor reduction, and/or increases overall survival of the subject as compared to injection of MVA alone.

Embodiment 24 is a recombinant MVA according to any one of embodiments 1-6, a vaccine according to embodiment 21, or a pharmaceutical combination according to any one of embodiments 7-9, for use in a method for stimulating an immune response in a subject, the method comprising intratumorally or intraperitoneally administering to the subject said recombinant MVA, vaccine, or pharmaceutical combination, wherein the intratumoral or intraperitoneal administration enhances an inflammatory response in the cancerous tumor that is detectable by analysis of the tumor or by analysis of blood or sera of the subject as compared to administration of MVA alone or as compared to a non-intratumoral or non-intraperitoneal administration of said recombinant MVA, or as compared to an intratumoral or intraperitoneal administration of a recombinant MVA lacking one or more of the components encoded by said recombinant MVA.

Embodiment 25 is a recombinant MVA according to any one embodiments 1-6, a vaccine according to embodiment 21, or a pharmaceutical combination according to any one of embodiments 7-9 for use in a method for treating cancer in subject.

Embodiment 26 is a recombinant MVA according to any one of embodiments 1-6, a vaccine according to embodiment 21, or a pharmaceutical combination according to any one of embodiments 7-9 for use in a method for treating cancer, wherein the cancer is selected from the group consisting of: breast cancer, lung cancer, head and neck cancer, thyroid, melanoma, gastric cancer, bladder cancer, kidney cancer, liver cancer, melanoma, pancreatic cancer, prostate cancer, ovarian cancer, urothelial, cervical, or colorectal cancer.

Embodiment 27 is a recombinant MVA according to any one of embodiments 1-6, wherein the enhanced inflammatory response is localized to the tumor.

Embodiment 28 is a method of inducing an enhanced inflammatory response in a peritoneal tumor of a subject, the method comprising intratumorally administering to the subject a recombinant modified Vaccinia Ankara (MVA) comprising a first nucleic acid encoding a first heterologous tumor-associated antigen (TAA) and a second nucleic acid encoding IL-12 or IL-12sc, wherein the intratumoral administration of the recombinant MVA generates an enhanced inflammatory response in the tumor as compared to an inflammatory response that would be generated by or would be expected to result from intratumoral injection of MVA virus alone.

Embodiment 29 is a method according to embodiment 28, wherein the MVA further comprises a nucleic acid encoding 4-1BBL.

Embodiment 30 is a recombinant modified Vaccinia Ankara (MVA), comprising: (a) a first nucleic acid encoding a tumor-associated antigen (TAA); and (b) a second nucleic acid encoding IL-12; wherein the intratumoral administration of the recombinant MVA enhances an inflammatory response in a tumor, reduces the growth rate and/or size of the tumor, and/or increases overall survival of the subject as compared to a non-intratumoral injection of said recombinant MVA or an injection of a recombinant MVA that does not comprise a nucleic acid encoding IL-12.

Embodiment 31 is the recombinant MVA of embodiment 30, further comprising (c) a third nucleic acid encoding 4-1BBL.

Embodiment 32 is the recombinant MVA of embodiment 30, wherein said TAA is an endogenous retroviral (ERV) protein.

Embodiment 33 is the recombinant MVA of embodiment 30, wherein said TAA is selected from the group consisting of carcinoembryonic antigen (CEA), mucin 1 cell surface associated (MUC-1), prostatic acid phosphatase (PAP), prostate specific antigen (PSA), human epidermal growth factor receptor 2 (HER-2), survivin, tyrosine related protein 1 (TRP1), tyrosine related protein 1 (TRP2), Brachyury, FOLR1, PRAME, HERV-K-env, HERV-K-gag, p15, and combinations thereof.

Embodiment 34 is a pharmaceutical combination comprising the recombinant MVA according to embodiment 30 and a pharmaceutically acceptable carrier.

Embodiment 35 is a pharmaceutical combination comprising the recombinant MVA according to embodiment 30 and a recombinant MVA comprising: (a) a first nucleic acid encoding a tumor-associated antigen (TAA); and (b) a second nucleic acid encoding 4-1BBL, wherein said TAA can be the same TAA as recited in claim 1 or can be a different TAA.

Embodiment 36 is a pharmaceutical combination according to embodiment 30, wherein said second recombinant MVA comprises a first nucleic acid encoding a TAA that is a different TAA than the one encoded by the recombinant MVA of embodiment 30.

Embodiment 37 is a method of stimulating an immune response in a subject having a plurality of tumors, comprising a step of locally (intratumorally) administering to fewer than all of the tumors in said subject a recombinant MVA comprising at least one first nucleic acid encoding a TAA and a second nucleic acid encoding IL-12, wherein an immune response to the TAA is stimulated in the subject.

Embodiment 38 is a method of treating a subject having at least one inaccessible tumor and at least one accessible tumor, comprising locally (intratumorally) administering to at least one accessible tumor in the subject a recombinant MVA comprising at least one first nucleic acid encoding a TAA and a second nucleic acid encoding 4-1-BBL, whereby the growth of the inaccessible tumor is decreased or stopped.

Embodiment 39 is a method of preventing or decreasing the extent of tumor recurrence or metastasis in a subject having at least one tumor, comprising intratumorally or intraperitoneally administering to at least one tumor in the subject a recombinant MVA comprising at least one first nucleic acid encoding IL-12 and optionally a second nucleic acid encoding a TAA, whereby the growth of the inaccessible tumor is decreased or stopped.

Embodiment 40 is the method of embodiment 37, 38, or 39, wherein said recombinant MVA further comprises a nucleic acid encoding 4-1BBL.

Embodiment 41 is a recombinant modified Vaccinia Ankara (MVA) for use in stimulating an immune response to a Tumor Associated Antigen (TAA) in a subject, comprising: (a) a first nucleic acid encoding IL-12, for example, scIL-12; and (b) a second nucleic acid encoding a TAA; wherein the intraperitoneal administration of the recombinant MVA enhances or increases an inflammatory response in a tumor, reduces the growth rate and/or size of the tumor, and/or increases overall survival of the subject as compared to a non-intraperitoneal injection of said recombinant MVA or an injection of a recombinant MVA that does not comprise a nucleic acid encoding IL-12, wherein the MVA is administered intraperitoneally.

Embodiment 42 is a recombinant MVA for use according to embodiment 41, further comprising (c) a third nucleic acid encoding 4-1BBL.

Embodiment 43 is a recombinant MVA for use according to embodiment 41, wherein said TAA is an endogenous retroviral (ERV) protein.

Embodiment 44 is a recombinant MVA for use according to embodiment 41, wherein said TAA is selected from the group consisting of carcinoembryonic antigen (CEA), mucin 1 cell surface associated (MUC-1), prostatic acid phosphatase (PAP), prostate specific antigen (PSA), human epidermal growth factor receptor 2 (HER-2), survivin, tyrosine related protein 1 (TRP1), tyrosine related protein 1 (TRP2), Brachyury, FOLR1, PRAME, HERV-K-env, HERV-K-gag, and combinations thereof.

Embodiment 45 is a recombinant MVA for use in the treatment of tumors, comprising: (a) a first nucleic acid encoding a tumor-associated antigen (TAA); and (b) a second nucleic acid encoding IL-12; wherein the intratumoral administration of the recombinant MVA enhances and/or increases an inflammatory response in a tumor, reduces the growth rate and/or size of the tumor, and/or increases overall survival of the subject as compared to a non-intratumoral injection of said recombinant MVA or an injection of a recombinant MVA that does not comprise a nucleic acid encoding IL-12; wherein the TAA is an endogenous retroviral (ERV) protein; and wherein the MVA is administered intratumorally.

Embodiment 46 is a recombinant MVA for use according to embodiment 45, wherein the TAA is selected from the group consisting of carcinoembryonic antigen (CEA), mucin 1 cell surface associated (MUC-1), prostatic acid phosphatase (PAP), prostate specific antigen (PSA), human epidermal growth factor receptor 2 (HER-2), survivin, tyrosine related protein 1 (TRP1), tyrosine related protein 2 (TRP2), Brachyury, FOLR1, PRAME, HERV-K-env, HERV-K-gag, and combinations thereof.

Embodiment 47 is a pharmaceutical combination comprising: (i) a recombinant modified Vaccinia Ankara (MVA), comprising: (a) a first nucleic acid encoding a tumor-associated antigen (TAA); and (b) a second nucleic acid encoding IL-12; wherein the intratumoral administration of the recombinant MVA enhances and/or increases an inflammatory response in a tumor, reduces the growth rate and/or size of the tumor, and/or increases overall survival of the subject as compared to a non-intratumoral injection of said recombinant MVA or an injection of a recombinant MVA that does not comprise a nucleic acid encoding IL-12; and (ii) a pharmaceutically acceptable carrier.

Embodiment 48 is a pharmaceutical combination comprising embodiment 7 and a recombinant MVA comprising: (a) a first nucleic acid encoding a tumor-associated antigen (TAA); and (b) a second nucleic acid encoding 4-1BBL, wherein said TAA can be the same TAA as recited in embodiment 47 or can be a different TAA.

Embodiment 49 is a pharmaceutical combination according to embodiment 48, wherein said second recombinant MVA comprises a first nucleic acid encoding a TAA that is a different TAA than the one encoded by the recombinant MVA of claim 41.

Embodiment 50 is a recombinant modified Vaccinia Ankara (MVA) for use in stimulating an immune response to a Tumor Associated Antigen (TAA) in a subject, comprising a nucleic acid encoding IL-12; wherein the intraperitoneal administration of the recombinant MVA increases an inflammatory response in a tumor, optionally a peritoneal tumor, and/or in the omentum, reduces the growth rate and/or size of the tumor, and/or increases overall survival of the subject as compared to a non-intraperitoneal injection of said recombinant MVA or an injection of a recombinant MVA that does not comprise a nucleic acid encoding IL-12, wherein the MVA is administered intraperitoneally.

### EXAMPLES

The following examples illustrate the invention but should not be construed as in any way limiting the scope of the claims.

### Example 1: Construction of Recombinant MVAs

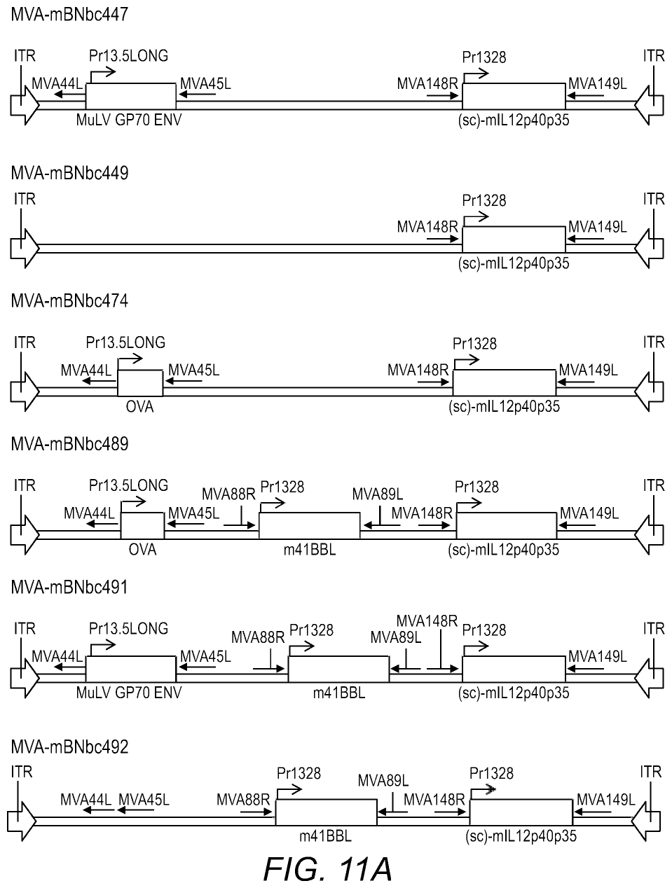

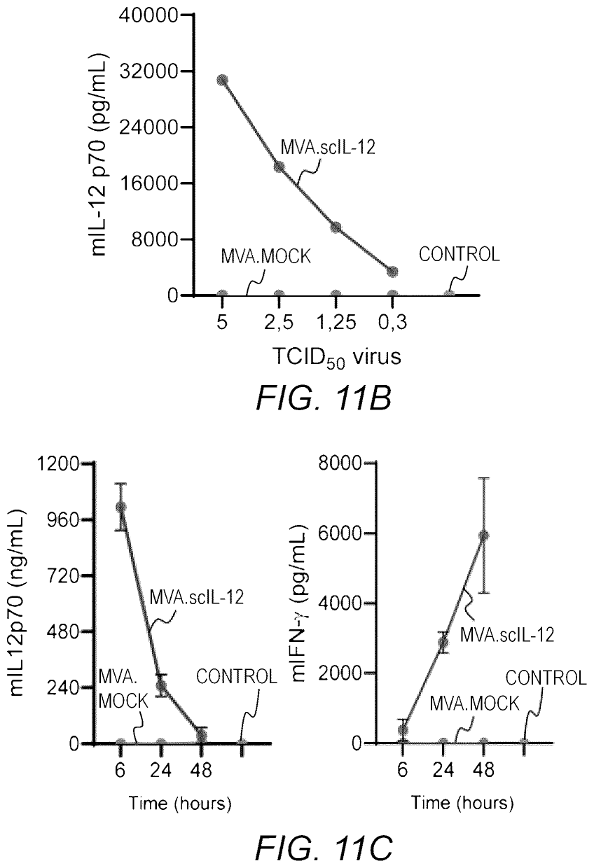

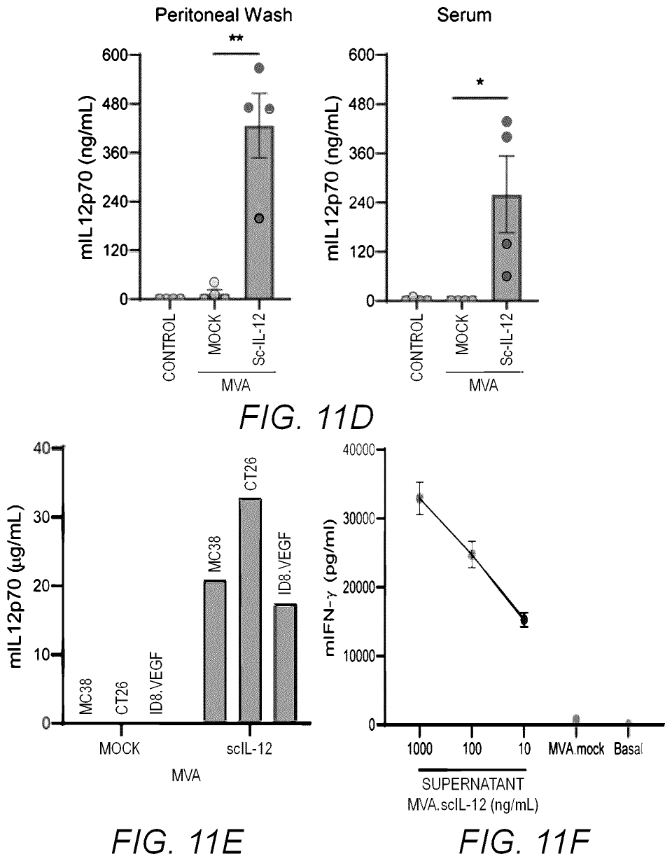

Schematic diagrams of the recombinant MVAs used in the experiments described herein are shown in FIG. 11A.

Recombinant MVAs encoding IL-12 (e.g., MVA-mIL12sc, encoding murine single chain IL-12), the model antigen OVA and IL-12 (MVA-OVA-mIL12sc) or the endogenous retroviral antigen Gp70 and IL12 (MVA-Gp70-mIL12sc) were produced (schematic diagrams of exemplary recombinant MVAs shown in FIG. 11A). Optionally, recombinant MVAs also encode 4-1BBL. Generally, recombinant MVAs encoding IL-12 or scIL-12 are referred to herein as “MVA-IL-12.”

Generation of recombinant MVA viruses that embody elements of the present disclosure was done by insertion of the indicated transgenes with their promoters into the vector MVA-BN, essentially as described previously.

MVA-mBNbc449 is a non-replicative, recombinant MVA encoding IL-12 without a tumor-associated antigen (diagrammed in FIG. 11A). Incubation of MVA.scIL-12 with mouse splenocytes induced the release of detectable amounts of scIL-12 into the supernatants in a dose-dependent manner. On the contrary, MVA.mock, an empty MVA vector (referred to herein as “MVA”), was unable to induce scIL-12 production in these immune cells. (Note subsequent experiments were performed on live model mice.) (FIG. 11B). Intraperitoneal administration of MVA-IL-12 but not MVA leads to IL-12 expression. Maximum levels were detected 6 h after vector administration, and at 48 h, IL-12 was undetectable in serum. IFN-γ induced by IL-12 was delayed, and maximum levels were detected 48 h after vector administration (FIG. 11C). At 6 h after administration, IL-12 was detected in both peritoneal wash and in serum when MVA-IL-12 was used, while IL-12 was not detected after MVA administration (FIG. 11D). On the other hand, MVA-IL-12 was able to infect tumor lines and release IL-12 in the supernatants (MC38, CT26, and ID8.Vegf) (FIG. 11E). Supernatants from MC38 in FIG. 11E were incubated with splenocytes demonstrating immunostimulatory activity by IFN-γ-inducing (FIG. 11F).

### Example 2: Systemic Inflammation Induced by Intratumoral Injection of Recombinant MVA Encoding IL-12

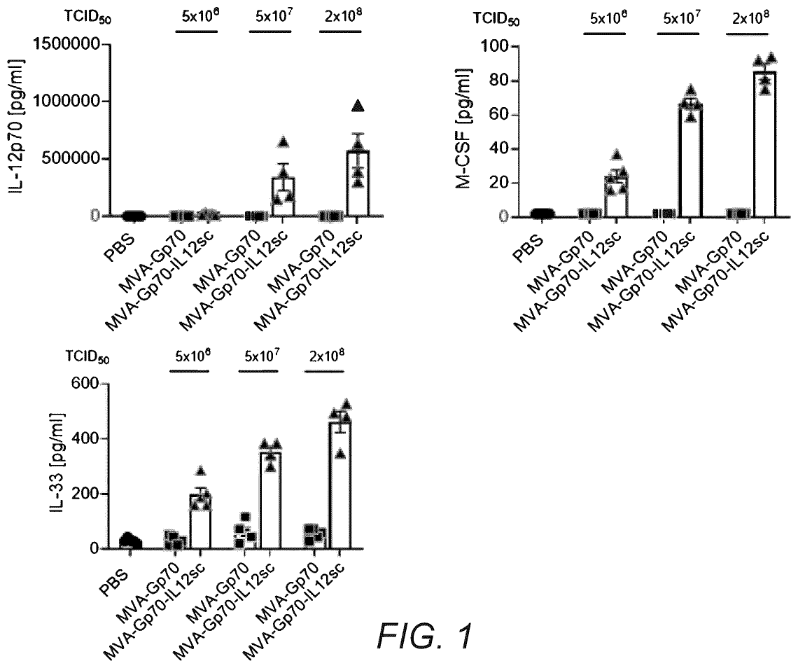

Local MVA-Gp70-IL12 injection results in systemic inflammation (see FIG. 1). C57BL/6 mice were inoculated subcutaneously with 5×10⁵ B16.F10 cells. When tumors were above 60 mm³ in volume, mice were grouped and injected intratumorally (IT) with either saline or increasing TCID₅₀ virus concentrations of either MVA-Gp70 or MVA-Gp70-IL12 (5×10⁶, 5×10⁷, or 2×10⁸ TCID₅₀ of each recombinant MVA shown in FIG. 1).

MVA-Gp70-IL12 response was titered in vivo by assessing the results of these intratumoral injections into the established B16.F10 melanomas. In contrast to membrane bound 4-1BBL, IL12sc is a soluble cytokine that is secreted upon expression. Mice were bled 6 hours after the first IT immunization and sera was analyzed for cytokine and chemokine expression (data shown in FIG. 1 as Mean±SEM). IL12p70 was detected when 5×10⁷ or 2×10⁸ TCID₅₀ MVA-Gp70-IL12 were injected intratumorally. Production of M-CSF and IL-33 was increased in a dose-dependent manner (FIG. 1). IL-33 is known to play many roles in inflammation (see, e.g., Le et al. (2013) Front. Immunol. 4:Art. 104 (1-9)).

### Example 3: Recombinant MVAs Expressing IL-12 Control Tumor Growth Following Intratumoral Injection

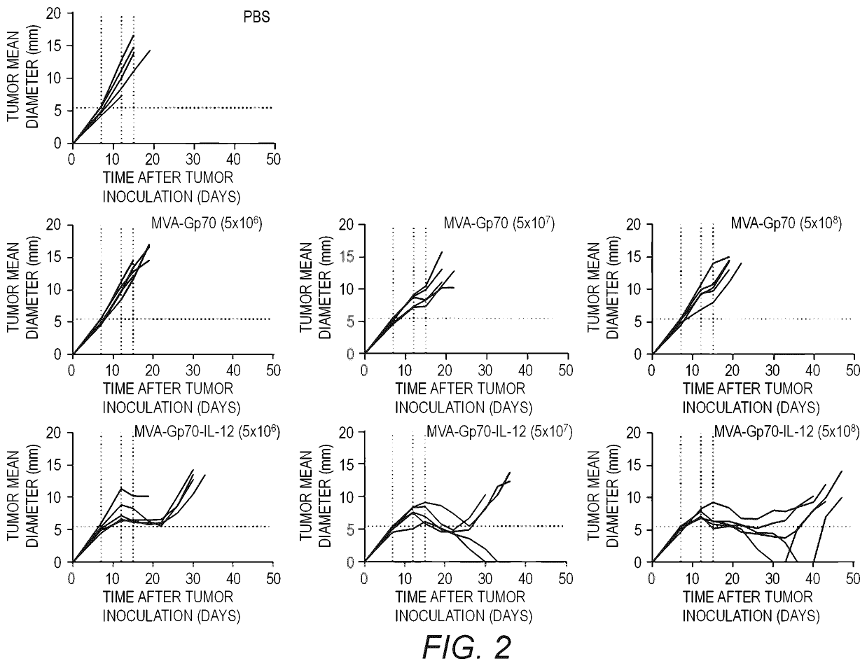

Mice were injected with recombinant MVAs encoding the model antigen gp70 or recombinant MVAs encoding both the model antigen gp70 and IL-12 (FIG. 2). Treatment with MVA-gp70 did not enhance tumor growth control, but injection of tumors with MVA-gp70-IL12 resulted in tumor growth control in all three doses tested (i.e., 5×10⁶, 5×10⁷, and 2×10⁸; FIG. 2).

Notably, two of the five mice that received 5×10⁷ TCID₅₀ MVA-Gp70-IL12 by IT injection cleared the B16.F10 tumors (FIG. 2). Tumor-bearing mice treated with injections of 2×10⁸ TCID₅₀ MVA-Gp70-IL12 showed signs of distress such as ruffled fur, weakness, and/or lack of appetite that could indicate drug-induced adverse immune effects, although further investigation would be needed to confirm this possibility. Because the 5×10⁷ TCID₅₀ dose of MVA-Gp70-IL12 resulted in mice that looked healthy and experienced the strongest therapeutic benefit after IT immunization, this dose was used in subsequent studies.

### Example 4: Combined Treatment with Recombinant MVAs Expressing IL-12 and 4-1BBL Provide Improved Control of Tumor Growth Following Intratumoral Injection

C57BL/6 mice were inoculated subcutaneously with 5×10⁵ B16.F10 cells. When tumors were above 60 mm³ in volume, mice were grouped and injected intratumorally (IT) with either saline, MVA-Gp70, MVA-Gp70-IL12, or both MVA-Gp70-IL12 and MVA-Gp70-4-1BBL at a dose of 5×10⁷ TCID₅O (i.e., a combination treatment; day of injection=Day 0). For the combination treatment, the injections comprised a 1:1 mix of MVA-Gp70-IL12sc with MVA-Gp70-4-1BBL. Mice received subsequent (“boost”) IT immunizations at days 5 and 8 (FIG. 3, vertical dotted lines). Tumor sizes were measured at regular intervals.

As observed in earlier experiments, repeated injection IT with MVA-Gp70-IL12 induced tumor growth control and rejection of 2 out of 5 B16.F10 melanomas (FIG. 3). Strikingly, the combination of recombinant MVAs expressing both 4-1BBL and IL12 cured 100% of the tested tumors. In this manner, local immunization with MVA-Gp70-IL12 combined with MVA-Gp70-4-1BBL induced complete rejection of these poorly immunogenic B16.F10 melanomas. Four out of five of the cured mice treated with this combination of MVAs developed drastic vitiligo (data not shown).

### Example 5: Treatment with Recombinant MVAs Expressing IL-12 or Combined with Recombinant MVAs Expressing 4-1BBL Induced Rejection of Colon Carcinomas

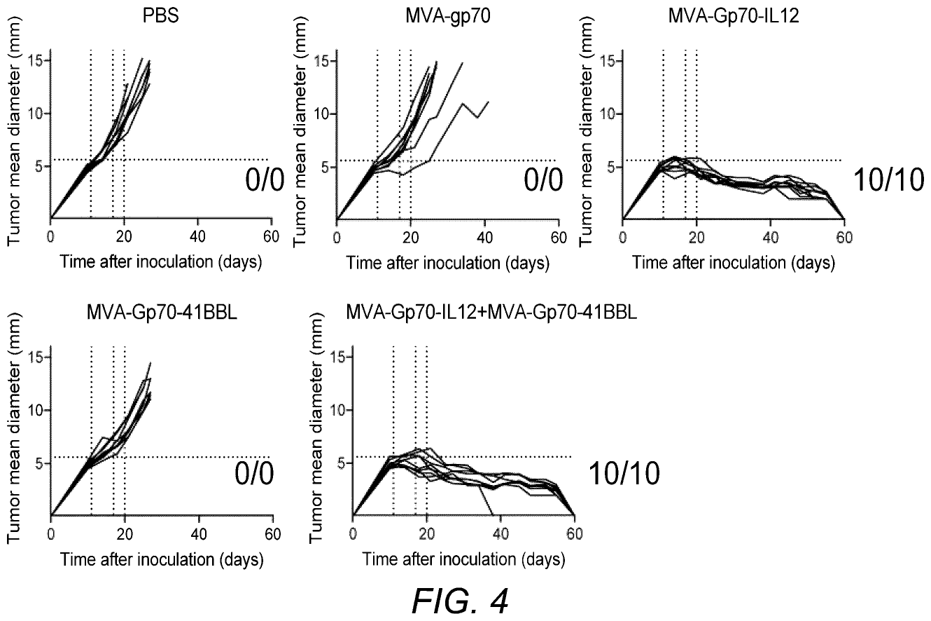

Results presented in FIG. 4 demonstrate that IT injection with MVA-Gp70-IL12 alone or combined with MVA-Gp70-4-1BBL induced rejection of MC38 colon carcinomas. C57BL/6 mice were inoculated subcutaneously with 5×10⁵ MC38 cells. When tumors were above 60 mm³ in volume, mice were grouped and injected intratumorally (IT) with either saline, MVA-Gp70, MVA-Gp70-4-1BBL, MVA-Gp70-IL12, or a combination of both 4-1BBL and 1112-expressing MVAs. All recombinant MVAs and combinations were administered at a dosage of 5×10⁷ TCID₅₀. Mice received additional (“boost”) IT immunizations at days 5 and 8 (vertical dotted lines). Tumors were measured at regular intervals. Number of cured mice is indicated in the lower right corner.

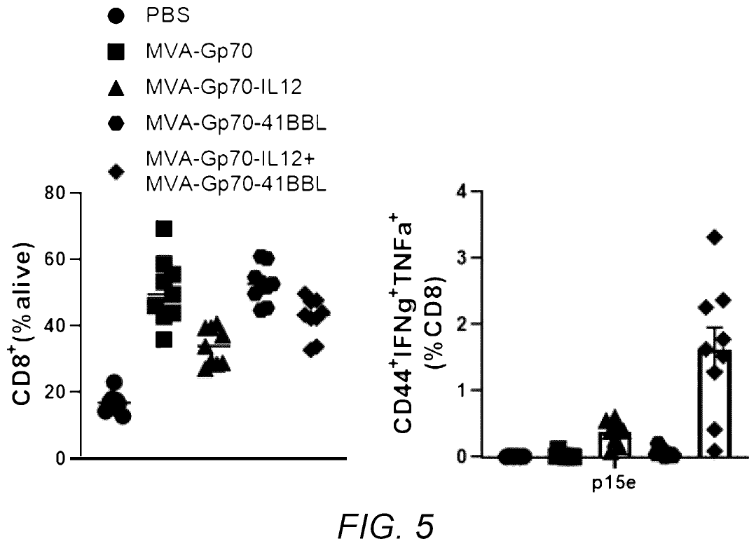

As shown in FIG. 4, MC38 tumors treated with either MVA-Gp70-IL12 or the combination of both MVA-Gp70-4-1BBL and MVA-Gp70-IL12 were completely eradicated. In addition, increased CD8⁺ T cell frequencies in the blood were observed for all treatment groups (FIG. 5). However, only the combination of recombinant MVAs encoding IL12 and 4-1BBL induced multi-cytokine-expressing CD8⁺ T cells upon peptide restimulation (FIG. 5).

### Example 6: Recombinant MVAs Encoding IL-12 Alone or in Combination with Recombinant MVAs Encoding 4-1BBL can Control Growth of Injected Tumors and Uninjected Tumors in the Same Subject

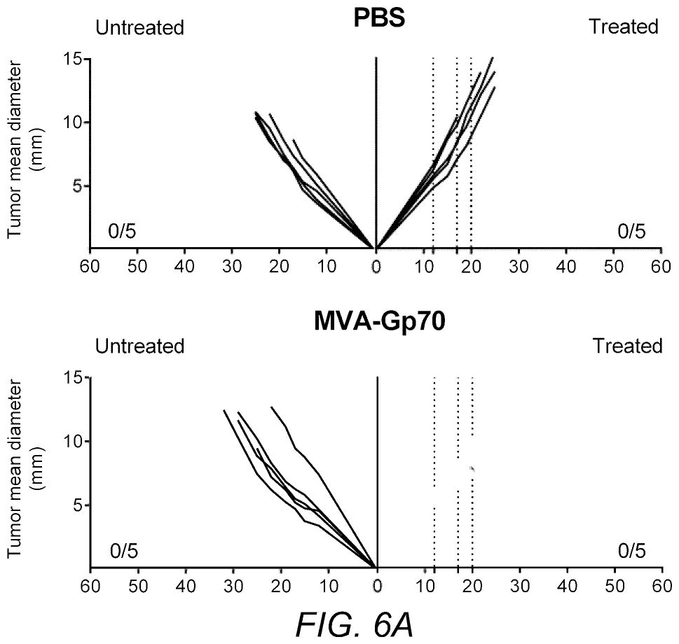

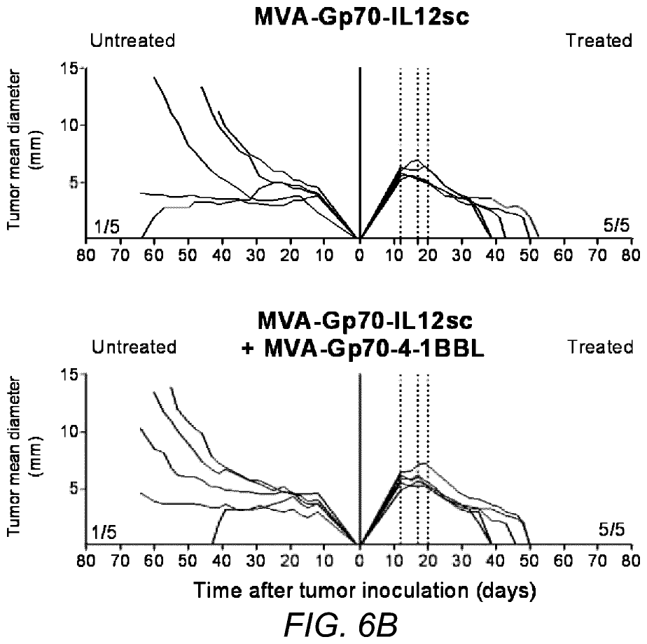

C57BL/6 mice were inoculated subcutaneously with 5×10⁵ MC38 cells on the right flank and 2×10⁵ on the left flank to produce mice with bilateral tumors. When right flank tumors were above 60 mm³ in volume, mice were grouped and injected intratumorally (IT) with either saline, MVA-Gp70, MVA-Gp70-4-1BBL, MVA-Gp70-IL12 or a combination of recombinant MVAs encoding 4-1BBL and recombinant MVAs encoding IL-12. All viruses and combinations were used at a TCID₅₀ of 5×10⁷. Mice received boost IT immunizations at days 5 and 8 (FIGS. 6A and 6B, vertical dotted lines). Tumor sizes were assessed at regular intervals (data presented in FIGS. 6A and 6B). Number of cured mice is indicated in the lower right corner in each figure panel.

Results (presented in FIGS. 6A and 6B) showed that treatment with PBS did not produce tumor growth control either in the treated or in the untreated tumor. As also seen in previous experiments (above), intratumoral injection of MVA-Gp70 induced a slight reduction in tumor growth in the treated tumor. Treatment with either MVA-Gp70-IL12 or the combination of MVA-Gp70-IL12 plus MVA-Gp70-4-1BBL resulted in tumor eradication of 5/5 treated tumor lesions and a drastic induction of tumor growth control of untreated tumors (see FIGS. 6A and 6B, showing that one of 5 untreated tumors was cured by both treatments including MVA-gp70-IL-12). However, only the combination treatment with MVAs encoding IL-12 and 4-1BBL induced multi-cytokine expressing CD8⁺ T cells upon peptide restimulation (data not shown).

### Example 7: Recombinant MVAs Encoding IL-12 Alone or in Combination with Recombinant MVAs Produced Rejection of Tumors on Rechallenge of Cured Mice

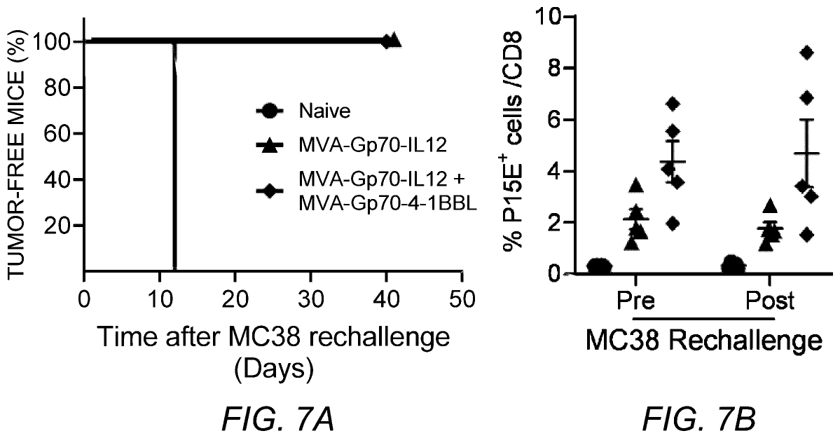

Naïve C57BL/6 mice and C57BL/6 mice that were cured of MC38 tumors following treatment with MVA-gp70-IL-12 alone or in combination with MVA-gp70-4-1BBL were rechallenged with MC38 tumor cells in the flank opposite to the one in which the primary tumor was placed. Tumor growth was measured at regular intervals. Results presented in FIGS. 7A and 7B show tumor free survival of mice over time (FIG. 7A) and percentage of tumor antigen-specific CD8⁺ T cells in blood pre and post rechallenge with the MC38 tumor cells (FIG. 7B). Data in FIG. 7B are expressed as Mean±SEM.

Interestingly, no differences in efficacy with regard to tumor occurrence were found between recombinant MVA encoding IL-12 and the combination of recombinant MVAs expressing IL-12 and 4-1BBL (FIG. 7A). However, blood peptide restimulation assays showed that multicytokine-producing, tumor-specific CD8⁺ T cells were expanded in the combination group (FIG. 7B).

### Example 8: Treatment with MVA-IL12 Induces Adaptive-Specific Immune Responses Against MC38 Colorectal Peritoneal Carcinomatosis

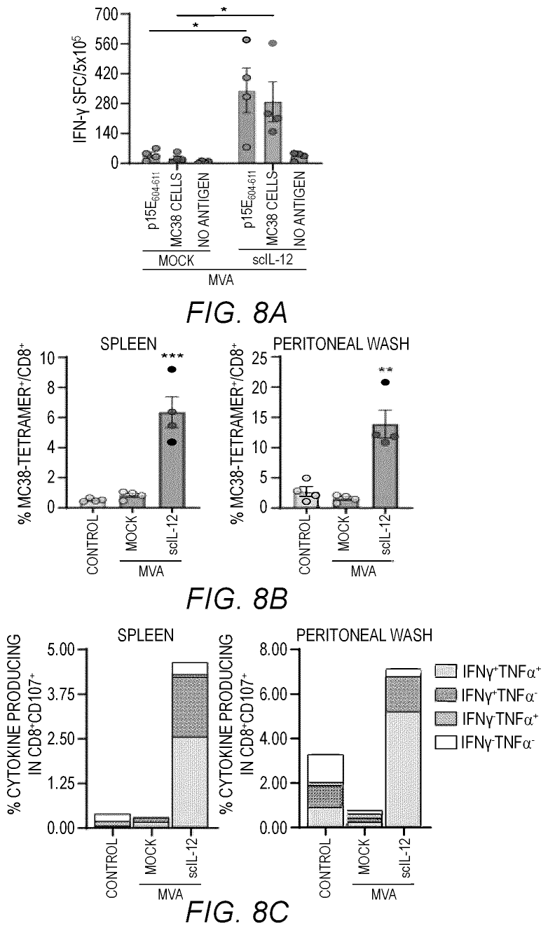

C57BL/6 mice (n=4) were challenged intraperitoneally (i.p.) with 5×10⁵ MC38 tumor cells. After seven days, subjects were treated i.p. with a single dose of 5×10⁷ TCID₅₀ (200 μl volume) of MVA alone (“MVA-BN”) as a control or recombinant MVA encoding IL-12 (“MVA-IL-12”). Seven days after injection, specific immune responses against MC38 tumor were analyzed. Mice were sacrificed humanely and the spleens were processed to isolate cells. An ELISpot assay was used to determine the number of T lymphocytes that produced IFN-γ in response to the endogenous retroviral p15E antigen expressed by the tumor cells and also against the MC38 tumor cells themselves. 5×10⁵ cells were incubated for 24 hours with either tumor-associated peptide KSPWFTTL (for stimulation of mouse MC38-specific CD8⁺ T cells), irradiated MC38 tumor cells (5×10⁴ cells, treated with 20,000 rads), or without antigen as a non-specific response. The frequency of number of IFN-γ specific spot forming cells (SFC) per 5×10⁵ cells was determined (FIG. 8A). Values are represented as mean±SEM. *p<0.05 (unpaired t test).

Although treatment with MVA alone did not increase the number of tumor-specific lymphocytes, administration of the MVA-IL-12 induced a high number of T lymphocytes specific to the tumor-associated antigen or against MC38 cells (FIG. 8A). CD8 lymphocytes were analyzed by flow cytometry in both the spleen and the peritoneal wash. MVA-IL-12 increased antigen-specific CD8⁺ cells (FIG. 8B) and the percentage of lytic CD107⁺CD8⁺ cells capable of producing both IFN-γ and TNF-α both locally and systemically (FIG. 8C).

### Example 9: Intraperitoneal Treatment with Recombinant MVA Encoding IL-12 Cured all Tested Mice Bearing MC38 Peritoneal Carcinomatosis and Provided Complete Protection after Tumor Rechallenge

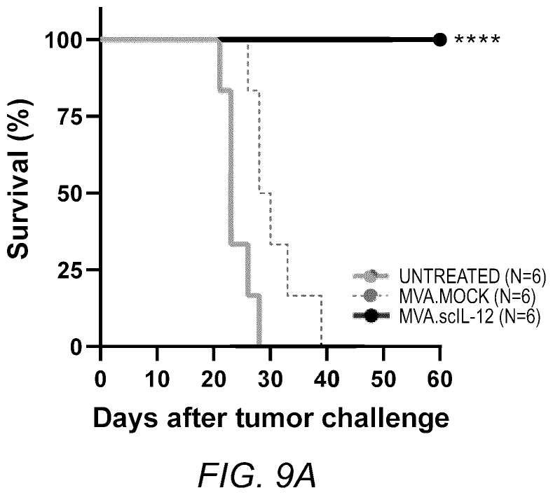

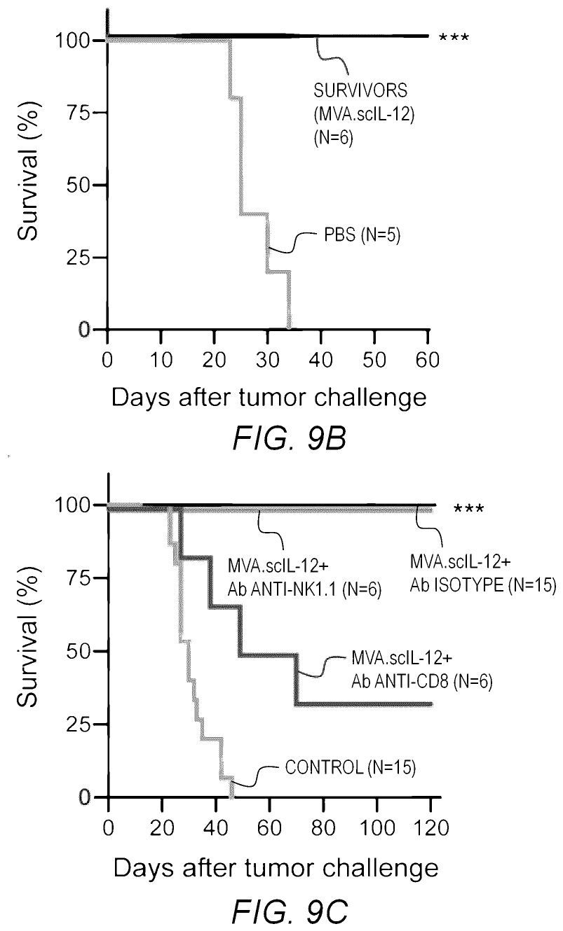

C57BL/6 mice (6 per group) were challenged i.p. with 5×10⁵ MC38 tumor cells as described above in Example 8. After seven days, mice were treated by intraperitoneal injection with a single dose of MVA alone (5×10⁷ TCID₅₀ in 200 μl volume) or recombinant MVA encoding IL-12; a third group was an untreated control. Survival was monitored daily The potent immune responses demonstrated in Example 8 were associated with strong antitumor responses that could be observed on Day 14 by the absence of hemorrhagic ascites and the lack of macroscopical tumor nodules in the peritoneum of mice treated with MVA-IL-12. Long-term follow-up of mice revealed large differences in survival as a function of treatment. PBS-treated mice (control group) died on day 30, control MVA-treated mice treated with MVA had a slight delay that was not significant, while all mice treated with the therapeutic vector eliminated tumor cells and survived 100% (FIG. 9A). Values are represented as Kaplan-Meier method (****p<0.0001, log-rank test).

To evaluate whether locoregional treatment elicited a systemic memory response, surviving mice that rejected MC38 peritoneal carcinomatosis after treatment with MVA.scIL-12 (i.e., “MVA-IL-12”) treatment (survivors, n=6) were rechallenged intraperitoneally (i.p.) with 5×10⁵ MC38 tumor cells. A naïve group was included as control group (n=5), and survival was monitored daily. All mice that had previously rejected the MC38 cell line rejected the rechallenge and survived until the end of the experiment. In contrast, the untreated control animals succumbed due to intraperitoneal tumor progression on day 40 (FIG. 9B).

To interrogate immune cells involved in the antitumor effect, depleting monoclonal antibodies against CD8 T lymphocytes or NK cells were used concomitantly with MVA-IL-12 (FIG. 9C). The antitumor effect was greatly diminished by CD8 depletion, although not completely abolished. The depletion of NK cells alone did not have any impact on overall survival. Therefore, CD8 T cells are required for the antitumor effect but act together with other immune cells to achieve maximum efficacy.

### Example 10: Injection of MVA-scIL12 Intraperitoneally is More Effective and Less Toxic than Intravenous Injection for Treating Peritoneal Carcinomatosis

Locoregional (i.p.) administration has different drawbacks, and its use must be clearly beneficial to the patient. Therefore, we compared this route of administration with the most common route of virus administration: intravenous (i.v.) injection.

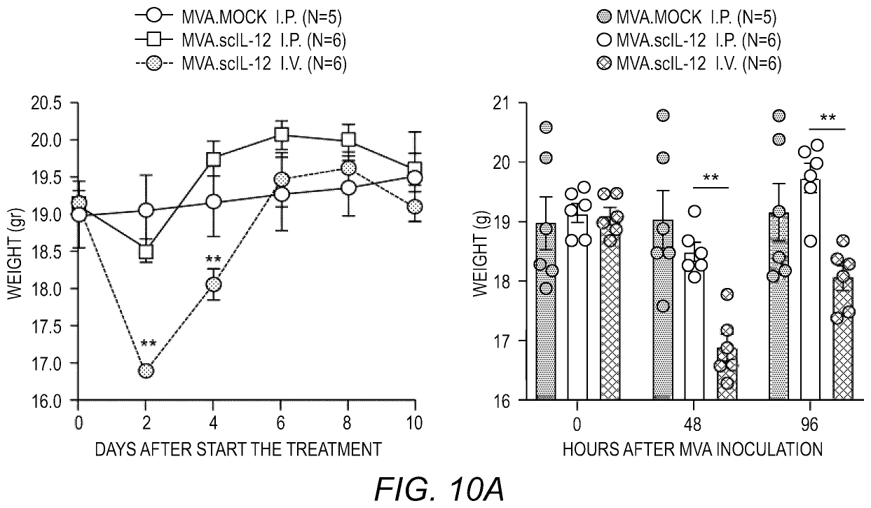

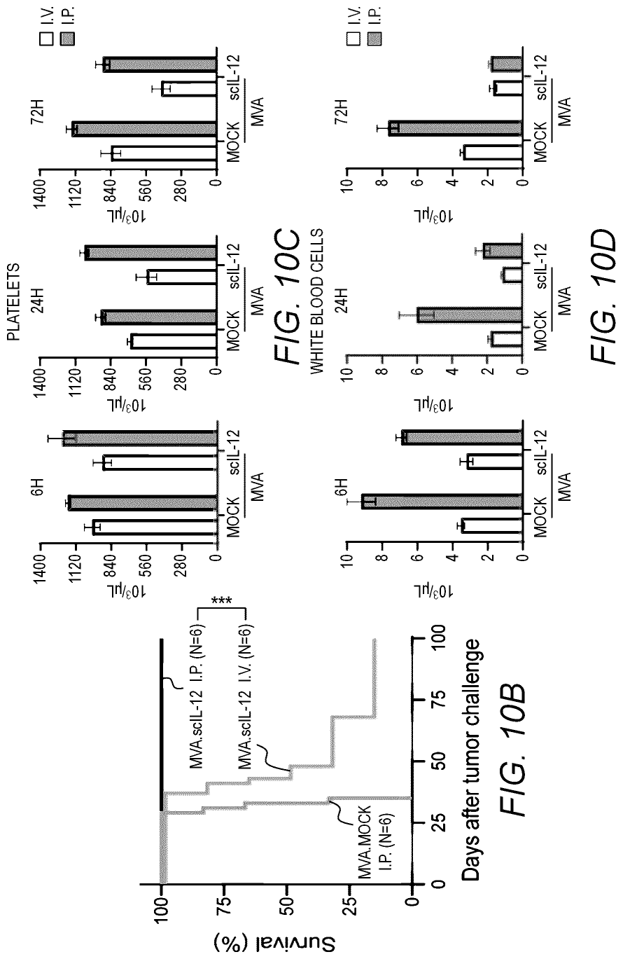

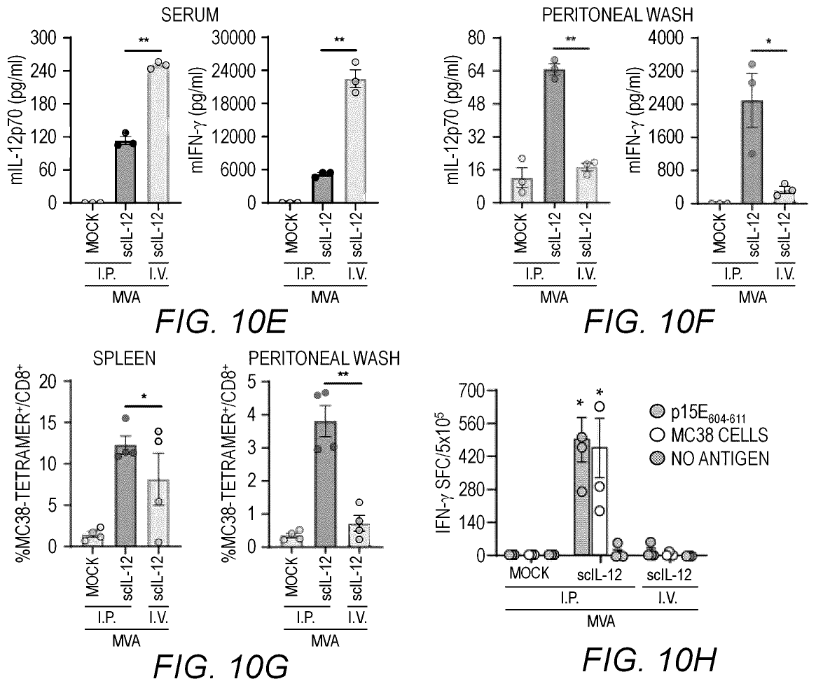

C57BL/6 mice (6 per group) were challenged intraperitoneally (i.p.) with 5×10⁵ MC38 tumor cells. After seven days, subjects were treated with a single dose of MVA alone by i.p. injection (5×10⁷ TC_{ID50} in 200 d volume), MVA-scIL-12 by i.p. injection, or MVA-scIL-12 by i.v. injection. Weight (in grams) was measured daily for up to 10 days following MVA administration (FIG. 10A, top panel), and showed an initial decrease for mice treated intravenously (FIG. 10A, bottom panel). Weight measurements in FIG. 10A are represented as mean±SEM (**p<0.01 (unpaired t test)). Survival status was confirmed daily, and survival is represented in FIG. 10B using the Kaplan-Meier method (****=p<0.0001, log-rank test). These results demonstrated that i.v. administration dramatically reduced the anti-tumor activity of MVA-IL-12.

Other effects of i.v. administration of MVA included hematologic toxicity, as reflected by reduced numbers 24 h and 72 h after administration in comparison to i.p. administration (FIG. 10C). The i.p. and i.v. route decreased the number of circulating white blood cells (FIG. 10D), possibly due to the up-regulation of the activation marker CD69 and subsequent inhibition of lymphocyte egress from lymphoid organs (Shiow et al. (2006) Nature 440: 540-4, “CD69 acts downstream of interferon-alpha/beta to inhibit S1P1 and lymphocyte egress from lymphoid organs”). While the invention is not bound by any particular mechanism of operation, to investigate the possible mechanism(s) for superiority of the i.p. route, we determined concentrations of scIL-12 and IFN-γ resulting from locoregional (i.p.) and systemic (i.v.) administration, IFN-γ being the main immune mediator induced by IL-12. MVA-IL-12 administered by the i.v. route induced very high levels of both cytokines systemically (as measured in serum), which may explain the previous toxic effects described (FIG. 10E). However, i.p. administration induced a dramatic increase in the concentrations of IL-12 and IFN-γ in peritoneal wash (FIG. 10F). I.p. administration also increased the percentage of tumor-specific CD8+ T cells systemically as well as in the peritoneum, while i.v. administration did not increase specific cell levels in the peritoneum and very variably increased levels in the spleen (FIG. 10G). Finally, using the ELISpot technique, we showed that i.p. administration was able to generate lymphocytes specific for both the particular antigen and entire tumor cells, while the i.v. route was not able to increase these levels in spleen (FIG. 10H).

### Example 11: MVA.scIL-12 Dose Escalation Improves Antitumor Effectiveness in Aggressive Models of Peritoneal Carcinomatosis

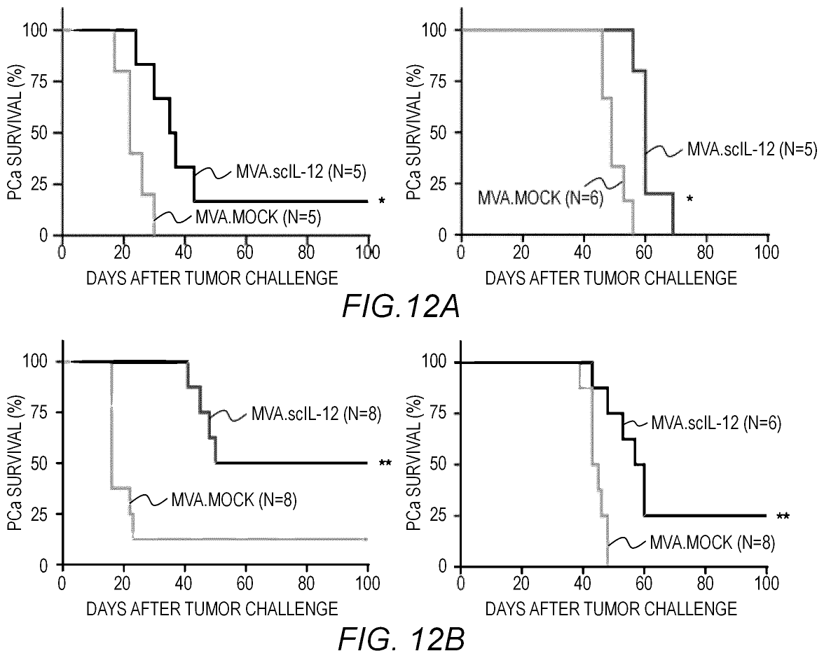

To confirm our results in other models of peritoneal carcinomatosis, mice were implanted intraperitoneally with CT26 colon cancer cells (2×10⁵ cells) or ID8.Vegf/GFP ovarian cancer cells (1×10⁶ cells) (=Day 0). In the group implanted with CT26 cells, mice were then intraperitoneally inoculated on Day 7 with 5×10⁷ TCID₅₀ of MVA or MVA-IL-12. In the group implanted with ID8.Vegf/GFP cells, mice were then intraperitoneally inoculated on Day 5 with 5×10⁷ TCID₅₀ of MVA or MVA-IL-12. Survival was then monitored and is shown in FIG. 12A for the CT26 group (left graph) and the ID8.Vegf/GFP group (right graph). In both the CT26 and ID8.Vegf/GFP groups, the model cancer cells were more resistant to treatment than the MC38 cell line used in some earlier experiments discussed herein.

Additional experiments were then conducted in which groups of mice were inoculated with CT26 and ID8.Vegf/GFP cells as above, but each group was then inoculated 3 times with doses of MVA or MVA-IL-12 (on days 7, 10, and 13 for the CT26 group and on days 5, 8, and 11 for the ID8.Vegf/GFP group; survival was monitored and is shown in FIG. 12B for the CT26 group (left graph) and the ID8.Vegf/GFP group (right graph). Results shown in FIGS. 12A and 12B demonstrate that although a single administration of MVA-IL-12 exerted a significant antitumor effect in both models of peritoneal carcinomatosis, the antitumor effect was improved by repeated administration of the MVA-IL-12 (3 doses). In the CT26 model (FIG. 12B, left graph), three doses of MVA-IL-12 delayed tumor death in all subjects, and 50% of subjects completely eradicated the tumors. In the ID8.Vegf/GFP model ovarian cancer, administration of 3 doses of MVA-IL-12 produced 25% tumor-free subjects at the end of the experiment. CT26-bearing mice treated with MVA-IL-12 were also confirmed to exhibit an increase in tumor-specific T lymphocytes, consistent with the increased survival shown in FIGS. 12A and 12B. These results highlight the antitumor effect of MVA-IL-12 in clinically relevant models of peritoneal carcinomatosis.

### Example 12: Locoregional (i.p.) Administration of MVA.scIL-12 Provides a Superior Antitumor Effect Over Intratumoral Administration

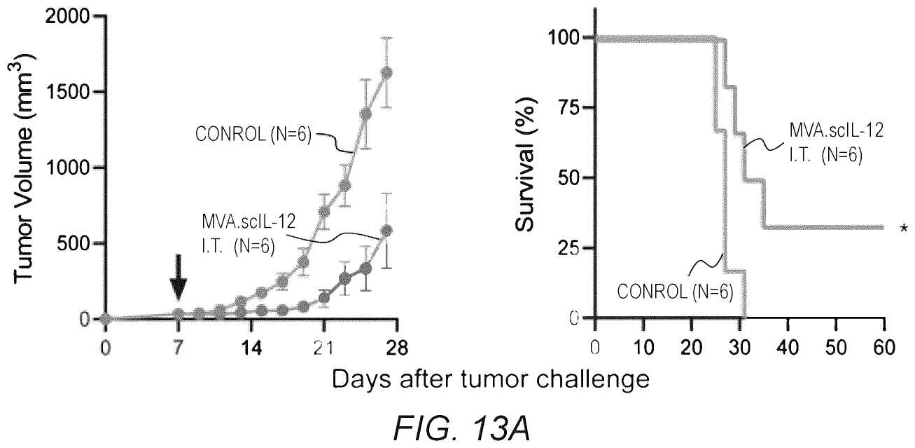

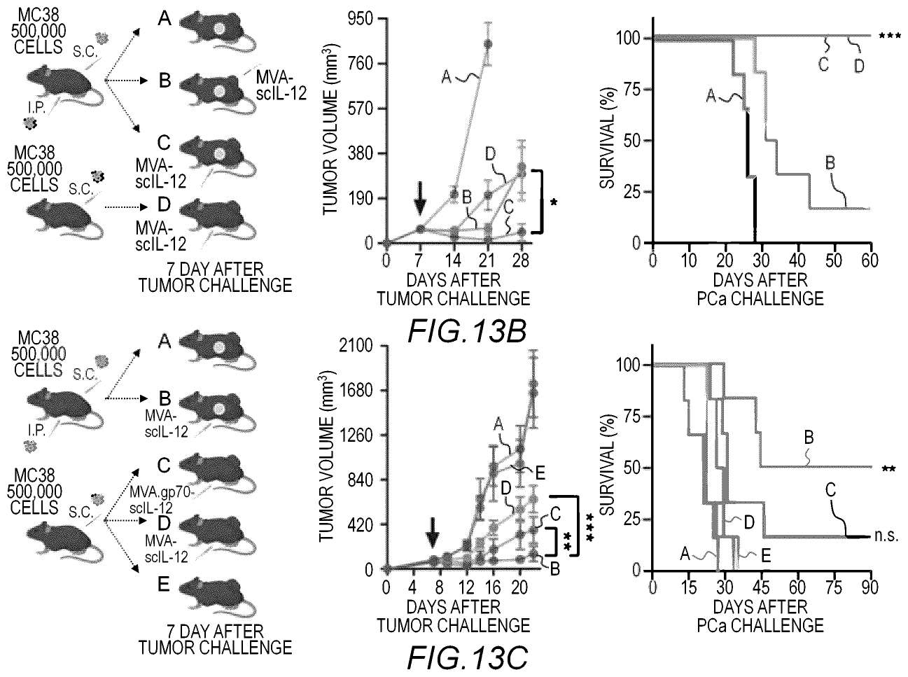

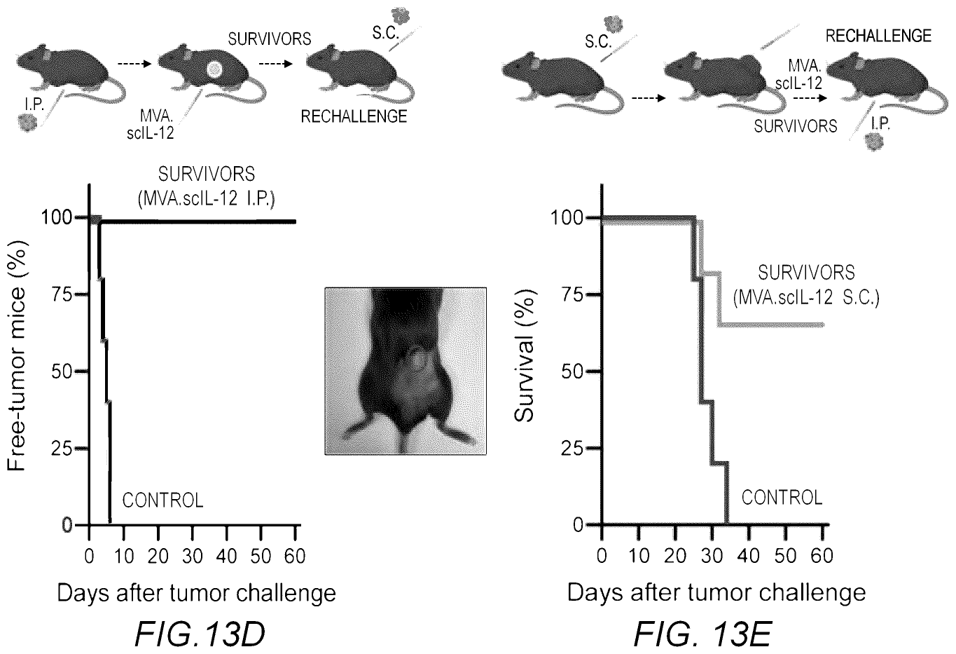

Experiments were conducted to compare the intraperitoneal (i.p.) and intravenous (i.v.) routes to the intratumoral (i.t.) route. Mice were injected subcutaneously (s.c.) with MC38 cells (5×10⁵ cells); 7 days later, MVA-IL-12 was administered to subjects intratumorally. This treatment had a significant effect on tumor growth, delaying the death of all mice and achieving a cure rate of approximately 30% (FIG. 13A). However, these results were inferior to those obtained by the i.p. route when MC38 lesions grow in the peritoneum, as demonstrated by previous experiments.

To further compare these routes of administration, experiments were designed to compare their local and systemic effects (FIG. 13B). Mice were injected with 5×10⁵ MC38 cells either subcutaneously (s.c.) or both s.c. and intraperitoneally (i.p.). Seven days after this tumor challenge, MVA-IL-12 was administered i.p. or i.t. (see FIG. 13B, left panel). Tumor volume and survival of subjects was monitored. Results showed that locoregional (i.p.) treatment of mice with i.p. and s.c. tumors exerted maximum efficacy, with a high percentage of mice cured of both tumors. Intracavitary administration also exerted control when the MC38 tumor was only growing subcutaneously. Finally, i.t. treatment in the s.c. and i.p. tumor model was only able to control the subcutaneous tumor, but had minimal effect on the peritoneal tumor.

As previously described, mice that eradicated the tumor in the peritoneum after i.p. treatment with MVA-IL-12 were able to eliminate MC38 cells injected subcutaneously in a rechallenge (FIG. 13D). However, of mice that eradicated the subcutaneous tumor after i.t. administration of MVA-IL-12, only 65% were able to reject a rechallenge with MC38 cells administered i.p. (FIG. 13E). Therefore, we can conclude that the i.p. route for administering MVA-IL-12 induces a greater local and distal immune response and is superior to other routes of administration.

To investigate the ability of a tumor in the peritoneum to initiate a systemic immune response, we generated an MVA vector that encoded both scIL-12 and the tumor-associated antigen gp70 (“MVA-gp70-IL-12,” e.g., the recombinant MVA-mBNbc447 diagrammed in FIG. 11). The antitumor effect of MVA-IL-12 on a subcutaneous tumor was evaluated (FIG. 13C). Using i.p. administration, the MVA-gp70-IL-12 exhibited an improved antitumor effect on this subcutaneous tumor compared to MVA-IL-12, while MVA-IL-12 treated tumors both in the peritoneum and in the flank of the animal (FIG. 13C).

### Example 13: Intraperitoneally Administered MVA Localizes in the Omentum

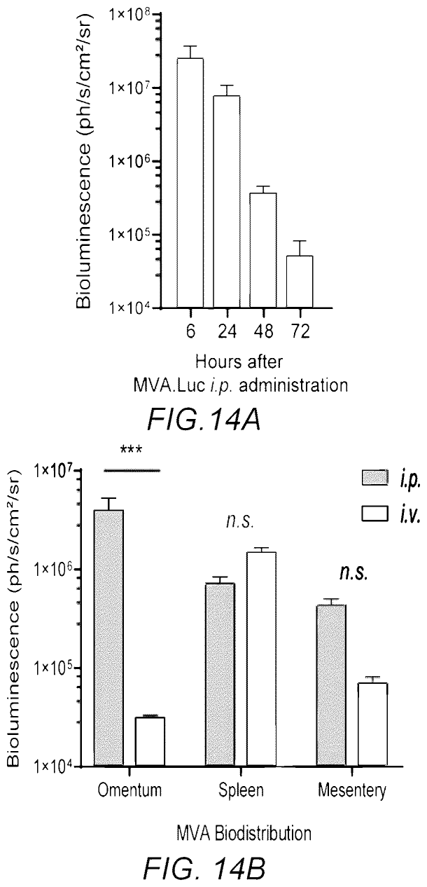

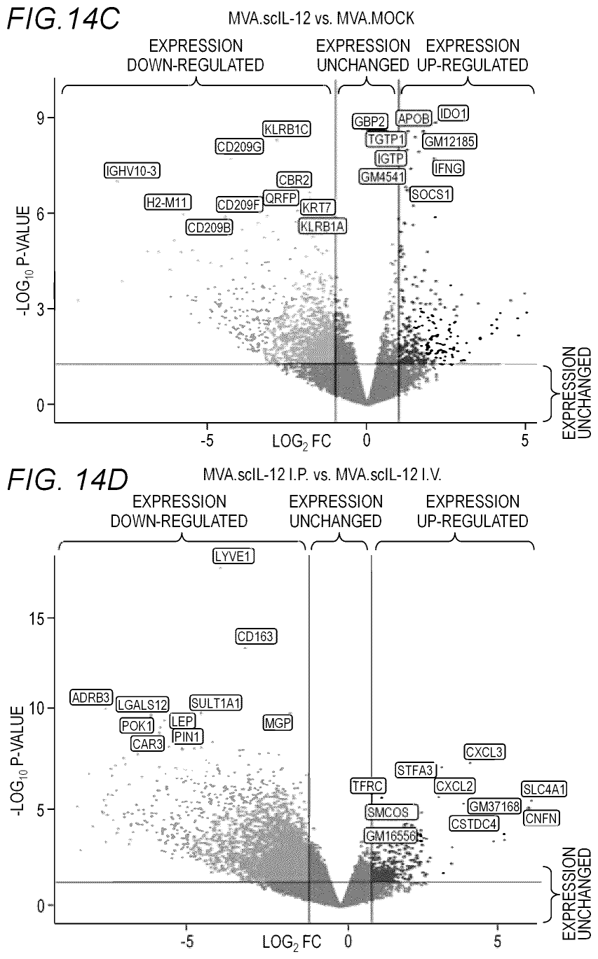

To further understand the reason for the superiority of the i.p. route of administration, we characterized the main transduced organs using an MVA encoding luciferase (Luc) after i.v. or i.p. administration in mice. The expression kinetics of MVA expressing luciferase (MVA-Luc) were similar to those of MVA-IL-12. Maximum luciferase expression was detected 6 h after i.p. administration and returned to baseline levels 72 h later (FIG. 14A). After i.v. injection, the organ with the most intense bioluminescence was the spleen. The signal in the omentum and mesentery was ten times less intense. In contrast, i.p. administration produced an intense signal in the omentum. The luminescence in the mesentery and spleen were ten times lower, but higher than 105 ph/s/cm2/sr (FIG. 14B). These results are highly relevant in peritoneal carcinomatosis since the omentum is the first organ to which tumor cells locate. In fact, macroscopic tumor nodules were observed in mice sacrificed on day 15 after MC38 inoculation. MVA-IL-12 administered i.v. reduced the tumor nodule size, but the most effective treatment was i.p. administered MVA-IL-12; in this case, no tumor nodules were visible, and enlarged milky spots indicated a powerful immune response elicited in this tissue.

These luminescence results were confirmed and extended using intravital microscopy to assess the effects of i.p. injection of MVA-IL-12 on the omenta of mice bearing ID8.Vegf/GFP tumors. hCD2RFP transgenic mice injected with ID8.Vegf/GFP tumor cells were examined by intravitral microscopy, which showed that the tumor cells localized in the omentum, mostly in the vicinity of fat-associated lymphoid clusters (FALCs), and increased in number and size over time.

The capacity of MVA to infect the omentum was also assessed using transcriptomic analysis of omenta from mice treated with MVA or MVA-IL-12. No significant differences were observed between mice infected with MVA and MVA-IL-12, suggesting that IL-12 expression does not affect virus infection. However, the expression of this proinflammatory cytokine impacted multiple cellular processes and modulated the expression of genes involved in immune responses. Administration of MVA-IL-12 induced a different transcriptomic profile than MVA (FIG. 14C), and transcriptomic profiles also differed between subjects treated with MVA-IL-12 intraperitoneally (i.p.) and intravenously (i.v.) (FIG. 14D). Transcriptomic analysis identified the up-regulation of several pathways involved in cellular metabolism and down-regulation of transcripts associated with macrophages and B cells.

### Example 14: Dose-Dependent Effects of Intratumoral Administration of MVA-Gp70-4-1BBL-IL12 in B16.F10 Melanoma-Bearing Mice

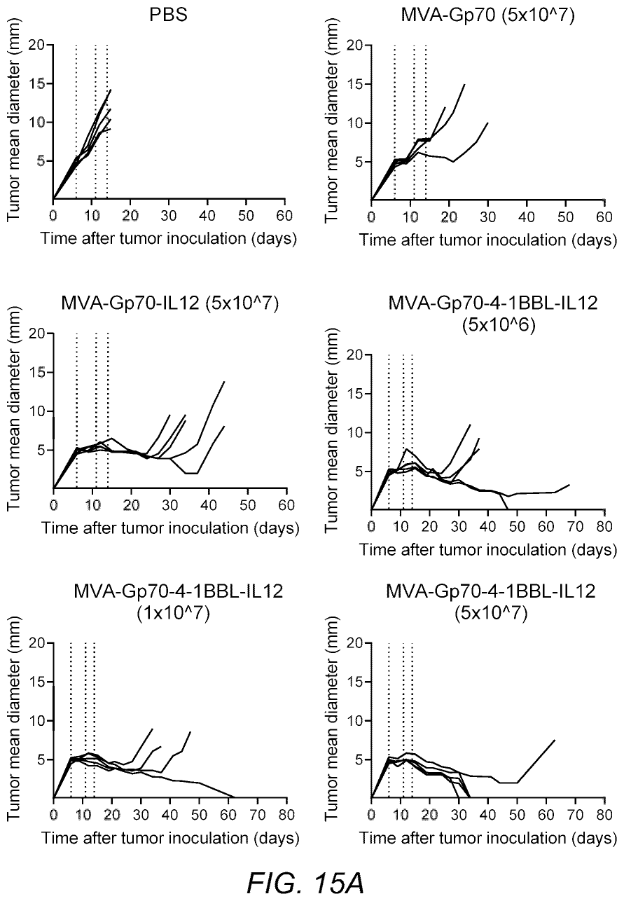

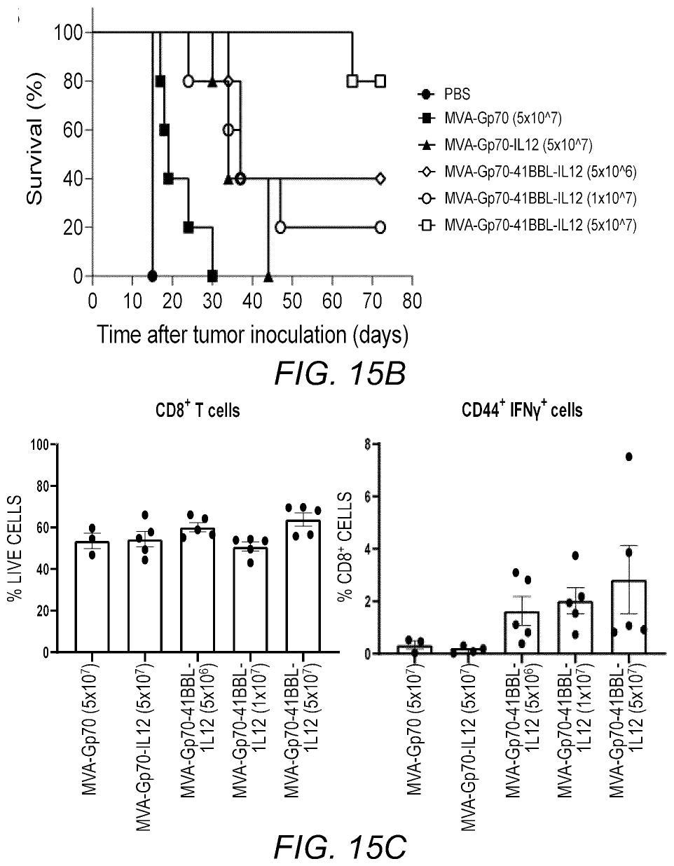

Treatment with recombinant MVA expressing a tumor-associated antigen (TAA) together with IL-12 and 4-1BBL (MVA-mBNbc491 (see FIG. 11)) provided tumor growth control and/or complete elimination of tumors following intratumoral injection, even when lower virus doses were used. C57BL/6 mice were inoculated subcutaneously with 5×10⁵ B16.F10 melanoma cells. When tumors were around 60 mm³ in volume, mice were grouped and injected intratumorally (i.t.) with either saline or 5×10⁷ TCID₅₀ MVA-Gp70 and MVA-Gp70-IL12, or with increasing doses of MVA-Gp70-4-1BBL-IL12 (at doses of 5×10⁶, 1×10⁷ and 5×10⁷ TCID₅₀). This first injection day was designated “Day 0.” Mice received subsequent (“boost”) i.t. immunizations at days 5 and 8 (FIG. 15, vertical dotted lines). Tumor sizes were measured at regular intervals.

Data showed that multiple i.t. administrations of MVA-Gp70-4-1BBL-IL12 were effective in inducing an anti-tumorigenic response, even at decreased doses. Low and medium doses of this recombinant MVA produced temporary tumor growth control and/or elimination of tumors. The strongest anti-tumorigenic effect was observed with the highest dose level, which resulted in complete elimination of tumors in four out of five mice and enhanced survival rate (FIG. 15A, 15B). Importantly, none of the mice treated with the highest dose displayed signs of distress, whereas all of them developed vitiligo.

Peripheral blood CD8+ T cell responses were examined four days after the last i.t. injection. Since all PBS-treated control mice were sacrificed at this time point due to aggressive tumor growth, no PBS control group was available for this analysis. No significant difference was observed between groups for total CD8+ T cell percentage. Importantly, MVA-Gp70-4-1BBL-IL12 induced CD44+ IFNγ expressing CD8+ T cells upon p15E peptide restimulation in a dose dependent manner (FIG. 15C).

### Example 15: Intratumoral (i.t.) Administration of MVA-Gp70-4-1BBL-1L12 Induces a Systemic Tumor-Specific Immune Response in B16.F10 Bilateral-Tumor-Bearing Mice

Experiments demonstrated that intratumoral injection of MVA-Gp70-4-1BBL-IL-12 induced a systemic anti-tumorigenic immune response in B16.F10 tumor bearing mice, which resulted in the control of both injected and uninjected distant tumors in the same animal.

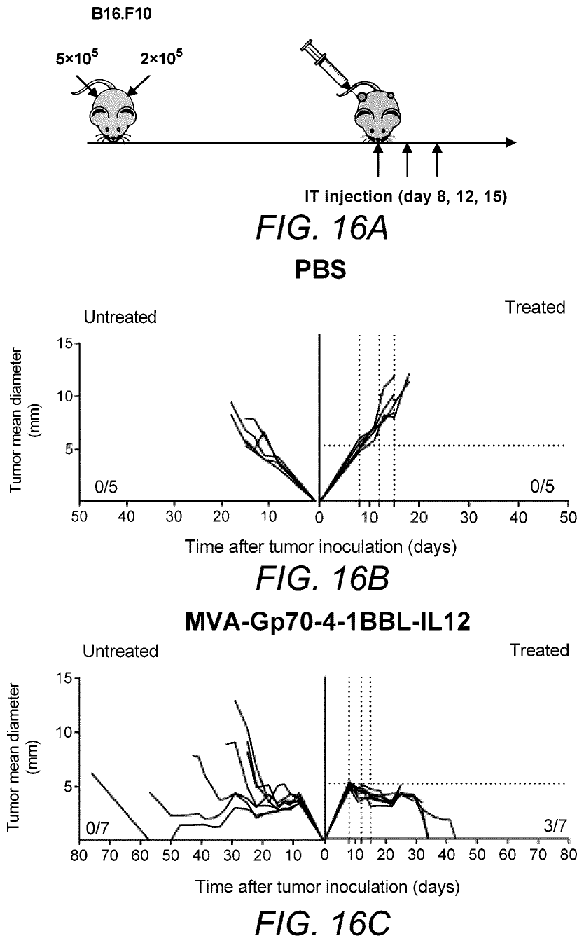

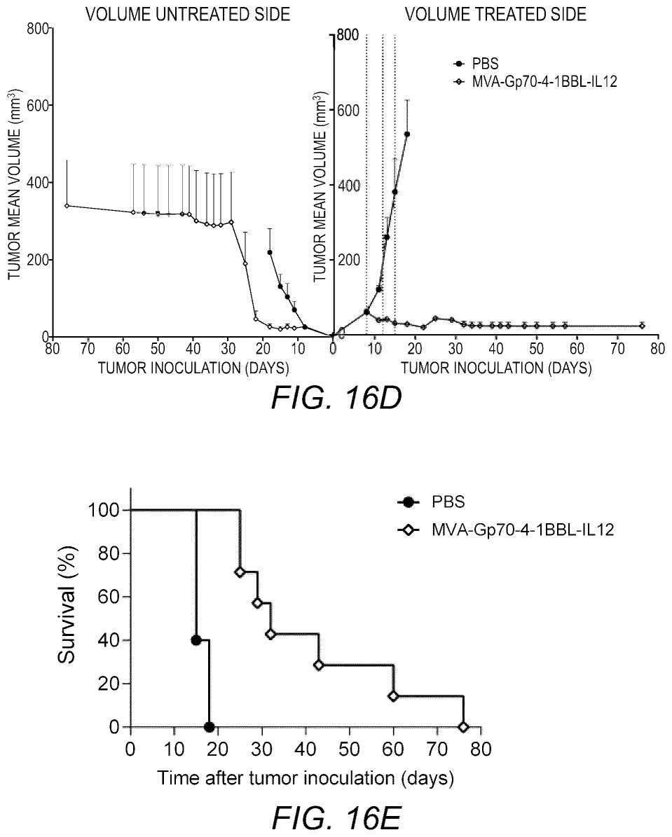

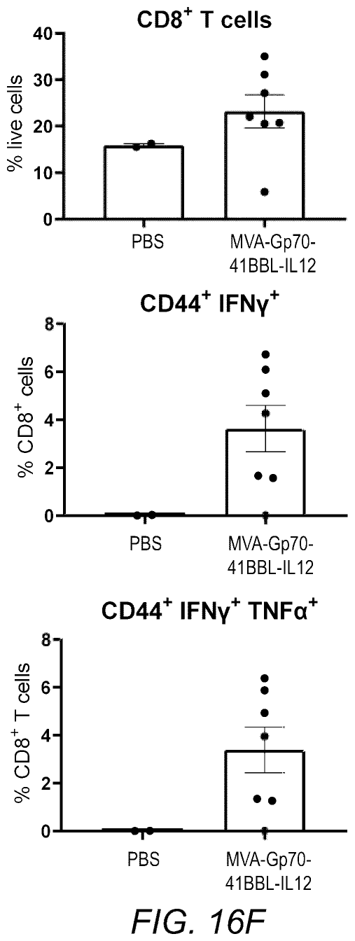

C57BL/6 mice were inoculated subcutaneously with 5×10⁵ and 2×10⁵ B16.F10 cells into the right and left flank, respectively (FIG. 16A) to produce mice with bilateral tumors. When right flank tumors were around 60 mm³ in volume, mice were grouped and injected intratumorally (i.t.) with either saline (PBS) or 5×10⁷ TCID₅₀ of MVA-Gp70-4-1BBL-IL12. Mice received additional (“boost”) i.t. immunizations on Day 4 and 7 (FIG. 16B, vertical dotted lines). Tumor sizes were assessed at regular intervals and the number of cured mice is indicated in the lower right corner in each figure panel.

Injection of PBS intratumorally did not result in tumor growth control on either of the treated or the untreated tumor, as expected, (FIG. 16B), whereas i.t. administration of MVA-Gp70-4-1BBL-IL12 resulted in growth control or shrinkage of both treated and untreated tumors as well as increased survival of the mice (FIGS. 16C, 16D, and 16E). Furthermore, the group treated with MVA-Gp70-4-1BBL-IL12 showed an increase in multi-cytokine-expressing CD8+T cells upon peptide restimulation (FIG. 16F). Overall, i.t. injection of MVA-Gp70-4-1BBL-IL12 induced a systemic anti-tumor response and increased the survival of B16.F10-tumor-bearing mice.

### Example 16: Systemic Anti-Tumor Effect is Induced by Intratumoral Administration of MVA-Gp70-4-1BBL-1L12 and does not Depend on NK Cells

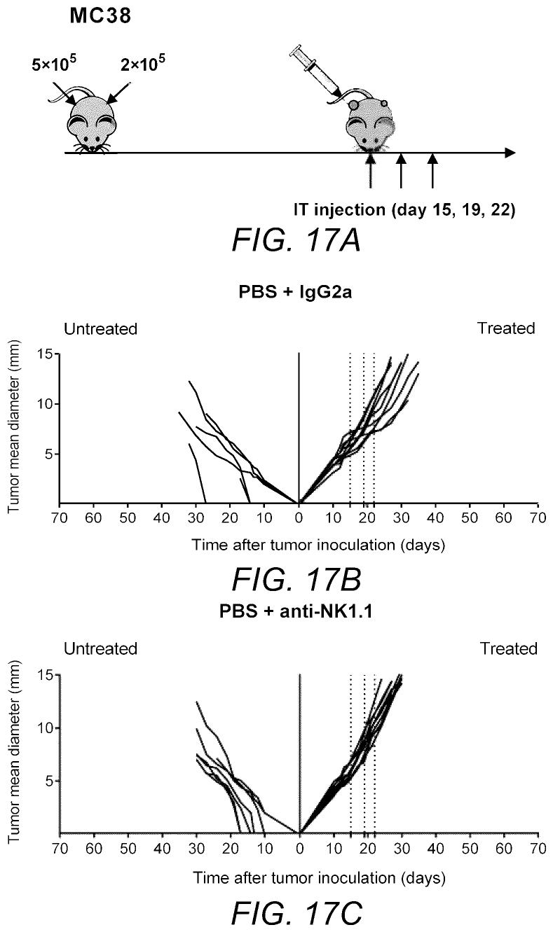

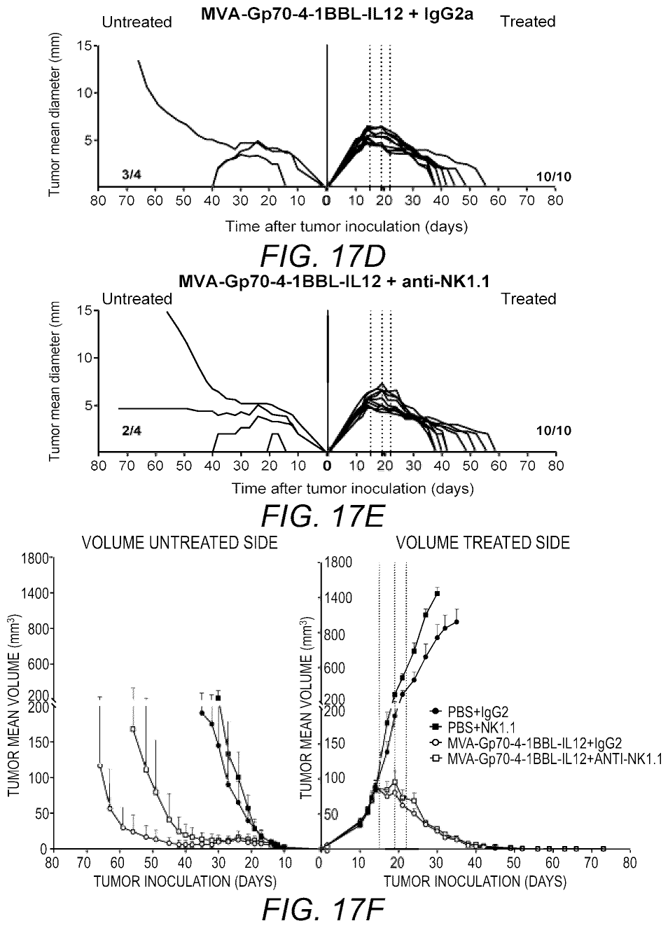

C57BL/6 mice were inoculated subcutaneously (s.c.) with 5×10⁵ and 2×10⁵ MC38 tumor cells into the right and left flank, respectively (FIG. 17A). Mice were grouped when tumors on the right flank were around 85-90 mm³ in size and received either anti-NK1.1 antibody or its isotype control IgG2a via intraperitoneal (i.p.) injections. One day after the i.p. injection, mice received either PBS or 5×10⁷ TCID₅₀ of MVA-Gp70-4-1BBL-IL12 via i.t. injection of the right tumor. Mice received additional (“boost”) i.t. immunizations at Days 4 and 7 (FIGS. 17B, 17C, 17D, and 17E, vertical dotted lines). Antibody/isotype injections were performed 2-3 times a week for 2½ weeks. Tumor sizes were assessed at regular intervals and the number of cured mice is indicated in the lower right corner in each figure panel.

Injection of PBS i.t. did not result in tumor growth control of either treated nor untreated tumors, whereas NK cell depletion accelerated tumor growth (FIGS. 17B and 17C). Importantly, i.t. administrations of MVA-Gp70-4-1BBL-IL12 induced a strong anti-tumor immune response against both treated and untreated tumors, which could be observed already after the 1^{st} i.t. immunization (FIG. 17D, indicated by dotted lines in the figures). Interestingly, the absence of NK cells did not affect the MVA-induced anti-tumorigenic response; irrespective of NK1.1 antibody depletion, tumors were eradicated after repetitive i.t. injection of MVA (FIG. 17E). Among these animals, 3 mice from the isotype-treated group and 2 mice from the NK1.1-antibody treated group eliminated both tumors and were entirely cured, showing that local administration of MVA-Gp70-4-1BBL-IL12 promoted a systemic tumor-specific immune response (FIGS. 17B, 17C, 17D, 17E, and 17F).

### Example 17: Repetitive Local Administration of MVA-Gp70-4-1BBL-1L12 Induced a Strong Tumor-Specific Immune Response, which is Partly Dependent on CD8⁺ Cells

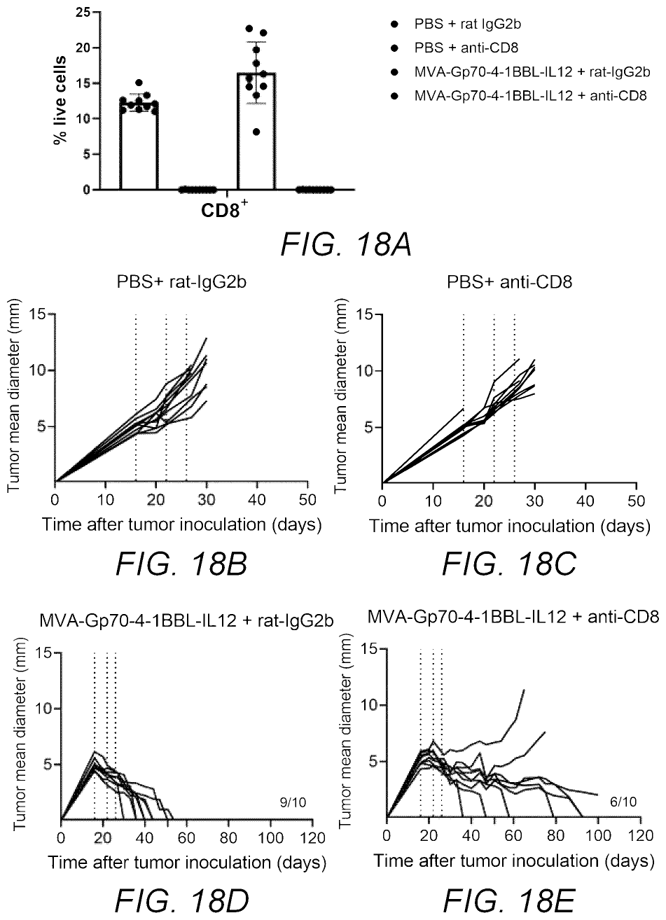

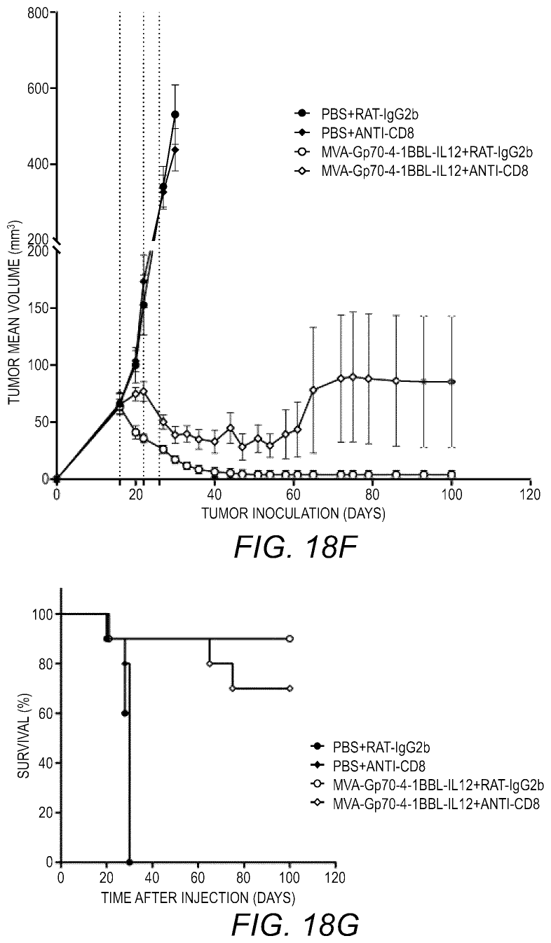

Anti-tumor effect induced by intratumoral MVA-Gp70-4-1BBL-IL12 administration is partly dependent on CD8+ T cells in the MC38 colon cancer tumor model. 5×10⁵ MC38 cells were subcutaneously (s.c.) injected into the right flank of mice. Mice were grouped when tumors were around 50 mm³ in volume, and they received i.p. injections of either anti-CD8 antibody or its isotype control IgG2b. After one day, mice received either PBS or 5×10⁷ TCID₅₀ of MVA-Gp70-4-1BBL-IL12 via i.t. injection of the tumor; this day was designated “Day 0.” Mice received additional (“boost”) i.t. immunizations on Days 6 and 10 (vertical dotted lines on the graphs). Antibody or isotype injections were performed in every 3-4 days for in total 35 days. Tumors were measured at regular intervals and the number of cured mice is shown (lower right corner of panels). The efficacy of CD8⁺ T cell depletion was shown five days after the first 1⁴ i.p. injection (FIG. 18A).

Results showed that injection of PBS did not induce tumor growth control either in CD8⁺ T cell sufficient or in depleted mice (FIG. 18B, 18C). Also as observed in previous experiments (above), repetitive i.t. injections of MVA-Gp70-4-1BBL-IL12 induced a strong anti-tumor effect right after the 1^{st} i.t. immunization and eventually 9 out of 10 mice eradicated the tumor completely (FIG. 18D). CD8⁺ T cell depletion had an impact on the efficacy of MVA-Gp70-4-1BBL-IL12 treatment compared to IgG2b group (FIG. 18E). Surprisingly, in the absence of CD8⁺ T cells, most of the mice showed a long lag phase of tumor growth, and 6 out of 10 mice were cured in the absence of CD8 T cells. Overall, i.t. injection of MVA-Gp70-4-1BBL-IL12 resulted in the shrinkage or complete elimination of MC38 tumors as well as increased survival of mice, and this effect was partly dependent on CD8⁺ T cells (FIG. 18F, 18G).

### Example 18: MVA-TAA-4-1BBL-IL12 Cured Mice are Resistant to Systemic Tumor Rechallenge

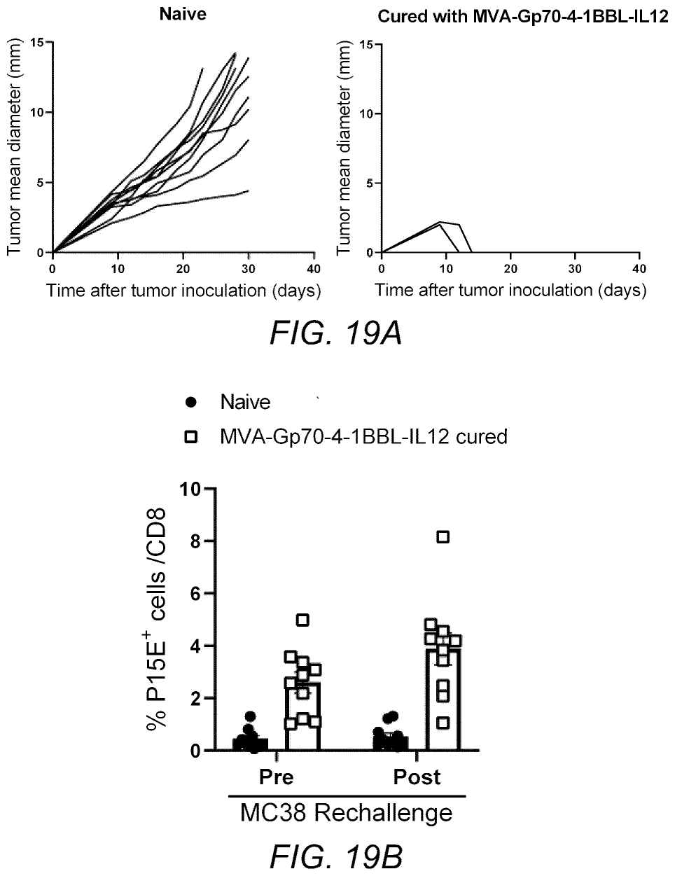

C57BL/6 mice that were cured of MC38 tumors following repetitive i.t. injection of MVA-Gp70-4-1BBL-IL12 were rechallenged with the same tumor cell line. Mice were subcutaneously (s.c.) injected with 5×10⁵ MC38 cells, and tumor growth was measured at regular intervals. Naïve C57BL/6 mice were used as control for tumor growth. While all naïve mice grew tumors, previously cured mice showed sporadic tumor growth but eventually eliminated all of these tumors, showing that previous i.t. injection of MVA-TAA-4-1BBL-IL12 was effective to induce tumor-specific memory response in treated mice (FIG. 19A). The percentage of antigen specific CD8⁺ T cells in the blood was observed in all cured mice before MC38 rechallenge after challenge cell percentages slightly increased in all cured mice (FIG. 19B).

It will be apparent that the precise details of the methods or compositions described herein may be varied or modified without departing from the spirit of the described invention. We claim all such modifications and variations that fall within the scope and spirit of the claims below.

### SEQUENCE LISTING

The nucleic and amino acid sequences listed below are shown using standard letter abbreviations for nucleotide bases, and either one letter code or three letter code for amino acids, as defined in 37 C.F.R. 1.822. Only one strand of each nucleic acid sequence is shown, but the complementary strand is understood as included by any reference to the displayed strand.

Relevant sequences:
SEQ ID NO: 1, mouse single chain IL-12 nucleotide sequence
((sc)-mIL-12p40p35) = recombinant murine single-chain
IL-12 with p40 and p35 subunits encoded)
ATGTGCCCTCAGAAGCTGACCATCAGTTGGTTCGCCATCGTGCTGCTGGTGTCCCCA
CTGATGGCTATGTGGGAACTCGAGAAGGACGTGTACGTGGTGGAAGTGGACTGGAC
CCCTGATGCTCCTGGCGAGACAGTGAACCTGACCTGCGACACACCTGAAGAGGACG
ACATCACCTGGACCAGCGATCAGAGACACGGCGTGATCGGCTCTGGCAAGACCCTG
ACAATTACCGTGAAAGAGTTCCTGGACGCCGGCCAGTACACCTGTCACAAAGGCGG
AGAGACACTGAGCCACTCTCATCTGCTGCTGCACAAGAAAGAGAACGGCATCTGGT
CCACCGAGATCCTGAAGAACTTCAAGAACAAGACCTTCCTGAAGTGCGAGGCCCCT
AACTACAGCGGCAGATTCACCTGTAGCTGGCTGGTGCAGAGAAACATGGACCTGAA
GTTCAACATCAAGTCCTCCAGCAGCAGCCCCGACAGCAGAGCTGTGACATGTGGCA
TGGCTAGCCTGAGCGCCGAGAAAGTGACACTGGACCAGAGAGACTACGAGAAGTAC
AGCGTGTCCTGCCAAGAGGACGTGACCTGTCCTACCGCCGAGGAAACACTGCCTAT
CGAGCTGGCCCTGGAAGCCAGACAGCAGAACAAATACGAGAACTACTCTACCAGCT
TCTTCATCCGGGACATCATCAAGCCCGATCCTCCAAAGAACCTGCAGATGAAGCCTC
TGAAGAACAGCCAGGTCGAGGTGTCCTGGGAGTACCCTGACAGCTGGTCTACCCCT
CACAGCTACTTCAGCCTGAAATTCTTCGTGCGGATCCAGCGCAAGAAAGAAAAGAT
GAAGGAAACCGAGGAAGGCTGCAACCAGAAAGGCGCTTTCCTGGTGGAAAAGACC
AGCACCGAGGTGCAGTGCAAAGGCGGCAATGTCTGTGTGCAGGCCCAGGACCGGTA
CTACAACAGCAGCTGTAGCAAGTGGGCCTGCGTGCCATGCAGAGTCAGATCTGGTG
GCGGAGGATCTGGCGGAGGTGGAAGCGGCGGAGGCGGATCTAGAGTGATTCCTGTG
TCTGGCCCTGCCAGATGCCTGAGCCAGTCTAGAAACCTGCTGAAAACCACCGACGA
CATGGTCAAGACCGCCAGAGAGAAGCTGAAGCACTACTCCTGCACAGCCGAGGACA
TCGATCACGAGGATATCACCAGGGACCAGACAAGCACCCTGAAAACCTGCCTGCCT
CTGGAACTGCATAAGAACGAGAGCTGCCTGGCCACCAGAGAAACCAGCTCTACCAC
AAGAGGCAGCTGTCTGCCTCCTCAGAAAACCAGCCTGATGATGACCCTGTGCCTGG
GCAGCATCTACGAGGATCTGAAGATGTACCAGACCGAGTTCCAGGCCATCAACGCC
GCTCTGCAGAACCACAACCACCAGCAGATCATCCTGGACAAGGGCATGCTGGTGGC
TATCGACGAGCTGATGCAGAGCCTGAACCATAACGGCGAGACACTGCGGCAGAAGC
CTCCAGTTGGAGAGGCCGATCCTTACAGAGTGAAGATGAAGCTGTGCATCCTGCTGC
ACGCCTTCAGCACCAGAGTGGTCACCATCAACAGAGTGATGGGCTACCTGAGCAGC
GCCTGA
SEQ ID NO: 2, mouse single chain IL-12 amino acid sequence
((sc)-mIL-12p40p35) = recombinant murine single-chain
IL-12 with p40 and p35 subunits encoded
MCPQKLTISWFAIVLLVSPLMAMWELEKDVYVVEVDWTPDAPGETVNLTCDTPEEDDI
TWTSDQRHGVIGSGKTLTITVKEFLDAGQYTCHKGGETLSHSHLLLHKKENGIWSTEILK
NFKNKTFLKCEAPNYSGRFTCSWLVQRNMDLKFNIKSSSSSPDSRAVTCGMASLSAEKV
TLDQRDYEKYSVSCQEDVTCPTAEETLPIELALEARQQNKYENYSTSFFIRDIIKPDPPKN
LQMKPLKNSQVEVSWEYPDSWSTPHSYFSLKFFVRIQRKKEKMKETEEGCNQKGAFLV
EKTSTEVQCKGGNVCVQAQDRYYNSSCSKWACVPCRVRSGGGGSGGGGSGGGGSRVI
PVSGPARCLSQSRNLLKTTDDMVKTAREKLKHYSCTAEDIDHEDITRDQTSTLKTCLPLE
LHKNESCLATRETSSTTRGSCLPPQKTSLMMTLCLGSIYEDLKMYQTEFQAINAALQNH
NHQQIILDKGMLVAIDELMQSLNHNGETLRQKPPVGEADPYRVKMKLCILLHAFSTRVV
TINRVMGYLSSA
SEQ ID NO: 3: mouse 4-1BBL nucleotide sequence
ATGGACCAGCACACACTGGACGTGGAAGATACCGCCGACGCCAGACACCCTGCCGG
CACAAGCTGTCCATCTGATGCCGCCCTGCTGAGAGACACAGGCCTGCTGGCTGATGC
TGCTCTGCTGTCTGACACCGTGCGGCCTACAAACGCCGCTCTGCCTACAGATGCCGC
CTACCCTGCTGTGAACGTGCGGGATAGAGAGGCCGCTTGGCCTCCCGCCCTGAACTT
CTGCAGCAGACACCCCAAGCTGTACGGCCTGGTGGCTCTGGTGCTCCTGCTGCTGAT
TGCCGCCTGCGTGCCCATCTTCACCAGAACCGAGCCTAGACCTGCCCTGACCATCAC
CACCAGCCCTAACCTGGGCACCAGAGAGAACAACGCCGACCAAGTGACCCCCGTGT
CCCACATCGGCTGCCCTAACACAACCCAGCAGGGCAGCCCTGTGTTCGCCAAGCTGC
TGGCCAAGAACCAGGCCAGCCTGTGCAACACCACCCTGAACTGGCACAGCCAGGAC
GGCGCTGGCAGCAGCTATCTGAGCCAGGGCCTGAGATACGAAGAGGACAAGAAAG
AACTGGTGGTGGACAGCCCTGGCCTGTACTACGTGTTCCTGGAACTGAAGCTGAGCC
CCACCTTCACCAACACCGGCCACAAGGTGCAGGGCTGGGTGTCACTGGTGCTGCAG
GCTAAGCCTCAGGTGGACGACTTCGACAACCTGGCCCTGACAGTGGAACTGTTCCCC
TGCAGCATGGAAAACAAGCTGGTGGATAGAAGCTGGTCCCAGCTGCTGCTGCTGAA
GGCCGGCCATAGACTGAGCGTGGGCCTGAGGGCTTATCTGCACGGCGCCCAGGACG
CCTACAGAGACTGGGAGCTGAGCTACCCCAACACAACCAGCTTCGGCCTGTTCCTCG
TGAAGCCCGACAACCCCTGGGAGTGA
SEQ ID NO: 4, mouse 4-1BBL amino acid sequence
MDQHTLDVEDTADARHPAGTSCPSDAALLRDTGLLADAALLSDTVRPTNAALPTDAAY
PAVNVRDREAAWPPALNFCSRHPKLYGLVALVLLLLIAACVPIFTRTEPRPALTITTSPNL
GTRENNADQVTPVSHIGCPNTTQQGSPVFAKLLAKNQASLCNTTLNWHSQDGAGSSYL
SQGLRYEEDKKELVVDSPGLYYVFLELKLSPTFTNTGHKVQGWVSLVLQAKPQVDDFD
NLALTVELFPCSMENKLVDRSWSQLLLLKAGHRLSVGLRAYLHGAQDAYRDWELSYP
NTTSFGLFLVKPDNPWE-
SEQ ID NO: 5, mouse gp70 nucleotide sequence
ATGGAAACCGACACACTGCTGCTGTGGGTGCTGCTTCTTTGGGTGCCCGGATCTACA
GGCGACGTGGCACTTGGAAACAGCCCTCACCAGGTGTTCAACCTGAGCTGGGAAGT
GACAAACGGCGACCGCGAAACAGTGTGGGCCATCACAGGCAATCACCCTCTGTGGA
CCTGGTGGCCTGACCTGACACCTGACCTGTGTATGCTGGCTCTGCACGGCCCATCTT
ACTGGGGCCTCGAGTACAGAGCCCCTTTCTCTCCTCCACCTGGACCTCCATGTTGTA
GCGGCAGCAGCGACAGCACAAGCGGCTGTTCTAGAGACTGCGAGGAACCCCTGACC
AGCTACACCCCTAGATGTAACACCGCCTGGAACAGACTGAAGCTGAGCAAAGTGAC
ACACGCCCACAACGAGGGCTTCTACGTGTGTCCTGGACCACACAGACCCAGATGGG
CCAGATCTTGTGGCGGCCCTGAGAGCTTCTACTGTGCTAGCTGGGGCTGCGAGACAA
CCGGCAGAGCTTCTTGGAAGCCTAGCAGCAGCTGGGACTACATCACCGTGTCCAAC
AACCTGACCTCCGACCAGGCTACCCCTGTGTGCAAGGGCAACAAGTGGTGCAACAG
CCTGACCATCAGATTCACCAGCTTCGGCAAGCAGGCCACCTCTTGGGTCACAGGACA
TTGGTGGGGCCTGAGACTGTATGTGTCCGGCCATGATCCTGGCCTGATCTTCGGCAT
CAGGCTGAAGATCACAGACAGCGGCCCCAGAGTGCCTATCGGCCCTAATCCTGTGC
TGAGCGACAGAAGGCCTCCTAGCAGACCCAGGCCTACAAGATCTCCACCTCCAAGC
AACAGCACCCCTACCGAGACACCTCTGACACTGCCTGAACCTCCACCAGCCGGCGT
GGAAAACAGACTGCTGAATCTGGTCAAGGGCGCCTACCAGGCTCTGAACCTGACCA
GCCCTGATAAGACACAAGAGTGCTGGCTGTGCCTGGTGTCTGGCCCTCCTTACTATG
AAGGCGTGGCCGTGCTGGGCACCTACAGCAATCATACAAGCGCCCCTGCCAACTGC
AGCGTGGCCTCTCAGCATAAGCTGACCCTGTCTGAAGTGACCGGCCAGGGCCTGTGT
ATTGGCGCTGTGCCTAAGACACACCAGGTGCTGTGCAACACAACCCAGAAAACCAG
CGACGGCAGCTACTACCTGGCTGCTCCTACAGGCACAACCTGGGCCTGTAGCACAG
GACTGACCCCTTGTATCAGCACCACCATCCTGAATCTGACCACCGACTACTGCGTGC
TGGTGGAACTGTGGCCTAGAGTGACCTACCACTCTCCTAGCTACGTGTACCACCAGT
TCGAGAGAAGGGCCAAGTACAAGCGCGAGCCCGTGTCTCTTACACTGGCCTTGCTTC
TCGGCGGCCTGACAATGGGAGGAATCGCTGCTGGTGTCGGCACCGGAACAACAGCT
CTGGTTGCCACACAGCAGTTCCAGCAGCTGCAGGCCGCTATGCACGACGACCTGAA
AGAGGTGGAAAAGAGCATCACCAACCTGGAAAAGTCTCTGACCAGCCTGAGCGAAG
TGGTGCTGCAGAACAGAAGAGGCCTGGACCTGCTGTTCCTGAAGCGCGGAGGACTG
TGCGCCTTCCTGAAAGAAGAGTGTTGCCTGTACGCCGACCACACCGGCCTCGTCAGA
GATTCTATGGCCAAGCTGAGAGAGAGACTGAGCCAGAGACAGAAGCTGTTCGAGTC
CCAGCAAGGATGGTTCGAGGGCCTGTTCAACAAGAGCCCCTGGTTCACCACACTGA
TCAGCACAATCATGGGCCCTCTGATCATTCTGCTGCTGATCCTCCTGTTTGGCCCCTG
CATCCTGAACAGGCTGGTGCAGTTCATCAAGGACAGAATCAGCGTGGTGCAGGCTC
TGGTGCTGACCCAGCAGTATCACCAGCTGAAAACCATCGGCGACTGCAAGAGCAGA
GAGTGA
SEQ ID NO: 6, mouse gp70 amino acid sequence
METDTLLLWVLLLWVPGSTGDVALGNSPHQVFNLSWEVTNGDRETVWAITGNHPLWT
WWPDLTPDLCMLALHGPSYWGLEYRAPFSPPPGPPCCSGSSDSTSGCSRDCEEPLTSYTP
RCNTAWNRLKLSKVTHAHNEGFYVCPGPHRPRWARSCGGPESFYCASWGCETTGRAS
WKPSSSWDYITVSNNLTSDQATPVCKGNKWCNSLTIRFTSFGKQATSWVTGHWWGLRL
YVSGHDPGLIFGIRLKITDSGPRVPIGPNPVLSDRRPPSRPRPTRSPPPSNSTPTETPLTLPEP
PPAGVENRLLNLVKGAYQALNLTSPDKTQECWLCLVSGPPYYEGVAVLGTYSNHTSAP
ANCSVASQHKLTLSEVTGQGLCIGAVPKTHQVLCNTTQKTSDGSYYLAAPTGTTWACS
TGLTPCISTTILNLTTDYCVLVELWPRVTYHSPSYVYHQFERRAKYKREPVSLTLALLLG
GLTMGGIAAGVGTGTTALVATQQFQQLQAAMHDDLKEVEKSITNLEKSLTSLSEVVLQ
NRRGLDLLFLKRGGLCAFLKEECCLYADHTGLVRDSMAKLRERLSQRQKLFESQQGWF
EGLFNKSPWFTTLISTIMGPLIILLLILLFGPCILNRLVQFIKDRISVVQALVLTQQYHQLKT
IGDCKSRE-
SEQ ID NO: 7, ovalbumin nucleotide sequence
ATGGGCTCCATCGGTGCAGCAAGCATGGAATTTTGTTTTGATGTATTCAAGGAGCTC
AAAGTCCACCATGCCAATGAGAACATCTTCTACTGCCCCATTGCCATCATGTCAGCT
CTAGCCATGGTATACCTGGGTGCAAAAGACAGCACCAGGACACAAATAAATAAGGT
TGTTCGCTTTGATAAACTTCCAGGATTCGGAGACAGTATTGAAGCTCAGTGTGGCAC
ATCTGTAAACGTTCACTCTTCACTTAGAGACATCCTCAACCAAATCACCAAACCAAA
TGATGTTTATTCGTTCAGCCTTGCCAGTAGACTTTATGCTGAAGAGAGATACCCAAT
CCTGCCAGAATACTTGCAGTGTGTGAAGGAACTGTATAGAGGAGGCTTGGAACCTA
TCAACTTTCAAACAGCTGCAGATCAAGCCAGAGAGCTCATCAATTCCTGGGTAGAA
AGTCAGACAAATGGGATTATCAGAAATGTCCTTCAGCCAAGCTCCGTGGATTCTCAA
ACTGCAATGGTTCTGGTTAATGCCATTGTCTTCAAAGGACTGTGGGAGAAAGCATTT
AAGGATGAAGACACACAAGCAATGCCTTTCAGAGTGACTGAGCAAGAAAGCAAACC
TGTGCAGATGATGTACCAGATTGGTTTATTTAGAGTGGCATCAATGGCTTCTGAGAA
AATGAAGATCCTGGAGCTTCCATTTGCCAGTGGGACAATGAGCATGTTGGTGCTGTT
GCCTGATGAAGTCTCAGGCCTTGAGCAGCTTGAGAGTATAATCAACTTTGAAAAACT
GACTGAATGGACCAGTTCTAATGTTATGGAAGAGAGGAAGATCAAAGTGTACTTAC
CTCGCATGAAGATGGAGGAAAAATACAACCTCACATCTGTCTTAATGGCTATGGGC
ATTACTGACGTGTTTAGCTCTTCAGCCAATCTGTCTGGCATCTCCTCAGCAGAGAGC
CTGAAGATATCTCAAGCTGTCCATGCAGCACATGCAGAAATCAATGAAGCAGGCAG
AGAGGTGGTAGGGTCAGCAGAGGCTGGAGTGGATGCTGCAAGCGTCTCTGAAGAAT
TTAGGGCTGACCATCCATTCCTCTTCTGTATCAAGCACATCGCAACCAACGCCGTTC
TCTTCTTTGGCAGATGTGTTTCCCCTTAA
SEQ ID NO: 8, ovalbumin amino acid sequence
MGSIGAASMEFCFDVFKELKVHHANENIFYCPIAIMSALAMVYLGAKDSTRTQINKVVR
FDKLPGFGDSIEAQCGTSVNVHSSLRDILNQITKPNDVYSFSLASRLYAEERYPILPEYLQ
CVKELYRGGLEPINFQTAADQARELINSWVESQTNGIIRNVLQPSSVDSQTAMVLVNAIV
FKGLWEKAFKDEDTQAMPFRVTEQESKPVQMMYQIGLFRVASMASEKMKILELPFASG
TMSMLVLLPDEVSGLEQLESIINFEKLTEWTSSNVMEERKIKVYLPRMKMEEKYNLTSV
LMAMGITDVFSSSANLSGISSAESLKISQAVHAAHAEINEAGREVVGSAEAGVDAASVSE
EFRADHPFLFCIKHIATNAVLFFGRCVSP-
SEQ ID NO: 9, human single-chain IL-12 nucleotide sequence
ATGTGTCACCAACAGCTGGTCATCAGCTGGTTCTCCCTGGTGTTCCTGGCCTCTCCTC
TGGTGGCCATCTGGGAGCTGAAGAAAGACGTGTACGTGGTGGAACTGGACTGGTAT
CCCGATGCTCCTGGCGAGATGGTGGTGCTAACCTGCGATACACCTGAAGAGGACGG
CATCACCTGGACACTGGATCAGTCTAGCGAGGTGCTCGGCTCTGGCAAGACCCTGAC
CATCCAAGTGAAAGAGTTTGGCGACGCAGGTCAGTACACCTGTCACAAAGGTGGAG
AAGTGCTGAGCCACAGCCTGCTGCTGCTCCACAAGAAAGAGGATGGCATTTGGAGT
ACCGACATCCTGAAGGATCAGAAGGAGCCTAAGAACAAGACCTTCCTGAGATGCGA
GGCCAAGAACTATAGTGGACGGTTCACATGTTGGTGGCTGACCACCATCAGCACCG
ACCTTACCTTCAGCGTGAAGAGCAGCAGAGGCAGCAGTGATCCTCAGGGAGTTACA
TGTGGTGCTGCTACACTGTCTGCCGAAAGAGTGAGAGGTGACAACAAGGAATACGA
GTACAGCGTGGAATGCCAAGAGGACAGCGCTTGTCCAGCTGCAGAAGAGTCTCTGC
CTATCGAAGTGATGGTGGACGCAGTGCACAAGCTGAAGTACGAGAACTACACCTCC
AGCTTCTTCATCAGAGACATCATCAAGCCTGATCCACCCAAGAACCTGCAGCTGAAG
CCTCTGAAGAACAGCAGACAGGTTGAAGTGTCCTGGGAGTACCCTGACACCTGGTCT
ACACCACACAGCTACTTCAGCCTGACCTTTTGCGTGCAAGTGCAGGGCAAGTCCAAG
CGAGAGAAGAAGGACCGTGTGTTCACCGACAAGACAAGCGCAACCGTGATCTGCAG
AAAGAACGCCAGCATCAGCGTCAGAGCCCAGGACCGGTACTACAGCAGCTCTTGGA
GCGAATGGGCAAGCGTGCCATGTTCTGGTGGTGGAGGATCTGGTGGAGGTGGAAGC
GGAGGAGGTGGATCTAGAAATCTGCCTGTGGCCACTCCTGATCCTGGCATGTTCCCT
TGTCTGCACCACAGCCAGAACCTTCTGAGAGCAGTGTCCAACATGCTCCAGAAGGC
CAGACAGACCCTGGAATTCTACCCATGCACCAGCGAGGAAATCGACCACGAGGACA
TCACTAAGGATAAGACCAGCACCGTGGAAGCCTGCCTGCCTCTGGAACTGACCAAG
AACGAGAGCTGCCTGAACAGCCGTGAAACCAGCTTCATCACCAACGGCTCTTGCCT
GGCAAGCAGGAAGACCTCCTTCATGATGGCTCTGTGCCTGAGCAGCATCTACGAGG
ACCTCAAGATGTACCAGGTGGAGTTCAAGACCATGAACGCCAAGCTGCTGATGGAC
CCTAAGCGGCAGATCTTCCTGGACCAGAATATGCTGGCAGTCATCGACGAGCTGATG
CAGGCACTGAACTTCAACAGCGAGACAGTGCCTCAGAAGTCTAGCCTGGAGGAACC
CGACTTCTACAAGACCAAGATCAAGCTGTGCATCCTGCTGCACGCCTTCCGTATCAG
AGCCGTGACCATCGACAGAGTGATGAGCTACCTGAACGCCTCCTGA
SEQ ID NO: 10, human single-chain IL-12 amino acid sequence
MCHQQLVISWFSLVFLASPLVAIWELKKDVYVVELDWYPDAPGEMVVLTCDTPEEDGI
TWTLDQSSEVLGSGKTLTIQVKEFGDAGQYTCHKGGEVLSHSLLLLHKKEDGIWSTDIL
KDQKEPKNKTFLRCEAKNYSGRFTCWWLTTISTDLTFSVKSSRGSSDPQGVTCGAATLS
AERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKT
SATVICRKNASISVRAQDRYYSSSWSEWASVPCSGGGGSGGGGSGGGGSRNLPVATPDP
GMFPCLHHSQNLLRAVSNMLQKARQTLEFYPCTSEEIDHEDITKDKTSTVEACLPLELTK
NESCLNSRETSFITNGSCLASRKTSFMMALCLSSIYEDLKMYQVEFKTMNAKLLMDPKR
QIFLDQNMLAVIDELMQALNFNSETVPQKSSLEEPDFYKTKIKLCILLHAFRIRAVTIDRV
MSYLNAS*
SEQ ID NO: 11, human single-chain IL-12 nucleotide sequence
(with linkage to the pre-mature p35, with short signal
peptide removed from the fusion protein)
ATGTGTCACCAGCAGCTGGTCATCAGCTGGTTCAGCCTGGTGTTCCTGGCCTCTCCTC
TGGTGGCCATCTGGGAGCTGAAGAAAGACGTGTACGTGGTGGAACTGGACTGGTAT
CCCGATGCACCTGGCGAGATGGTGGTGCTGACCTGCGATACACCTGAAGAGGACGG
CATCACCTGGACACTGGACCAGTCTAGCGAGGTGCTCGGCTCTGGCAAGACCCTGA
CCATCCAAGTGAAAGAGTTTGGCGACGCTGGACAGTACACCTGTCACAAAGGTGGA
GAAGTGCTGAGCCACAGCCTGCTGCTGCTCCACAAGAAAGAGGATGGCATTTGGTC
CACCGACATCCTGAAGGACCAGAAAGAGCCCAAGAACAAGACCTTCCTGAGATGCG
AGGCCAAGAACTACTCCGGACGGTTCACATGTTGGTGGCTGACCACCATCAGCACC
GACCTGACATTCAGCGTGAAGAGTAGCAGAGGCAGCAGTGATCCTCAGGGAGTTAC
ATGTGGAGCAGCTACACTGTCTGCCGAAAGAGTGAGAGGTGACAACAAAGAATACG
AGTACAGCGTGGAATGCCAAGAGGATAGTGCCTGTCCAGCAGCAGAAGAGTCTCTG
CCTATCGAAGTGATGGTGGACGCTGTGCACAAGCTGAAGTACGAGAACTACACATC
CAGCTTCTTCATCCGAGACATCATCAAACCAGATCCTCCCAAGAATCTGCAGCTGAA
GCCTCTGAAGAACAGCAGACAAGTGGAAGTGTCCTGGGAGTACCCAGACACCTGGT
CTACACCTCACAGCTACTTCTCCCTGACCTTTTGCGTGCAAGTGCAGGGCAAGTCCA
AGAGAGAGAAGAAGGACAGAGTCTTCACCGACAAGACATCTGCCACCGTGATCTGC
AGAAAGAACGCCAGCATCAGCGTCAGAGCCCAGGACCGGTACTACAGCAGCTCTTG
GAGCGAATGGGCAAGCGTGCCATGTTCTGGTGGTGGAGGATCTGGAGGAGGTGGAA
GCGGTGGAGGAGGATCTAGACCTGTTAGCCTGCAGTGCAGACTGAGCATGTGCCCA
GCTAGATCTCTCCTGCTGGTTGCCACACTGGTGCTCCTGGATCATCTGAGCCTGGCC
AGAAACCTGCCAGTGGCCACGCCTGATCCTGGCATGTTTCCTTGTCTGCACCACAGC
CAGAACCTGCTGAGAGCCGTTTCCAACATGCTGCAGAAGGCCAGACAGACCCTGGA
ATTCTACCCATGCACCAGCGAGGAAATCGACCACGAGGACATTACCAAGGATAAGA
CCAGCACCGTGGAAGCCTGCCTGCCTCTGGAACTGACCAAGAACGAGAGCTGCCTG
AACAGCCGTGAAACCAGCTTCATCACCAACGGCTCTTGCCTTGCCTCCAGGAAGACC
TCCTTCATGATGGCACTGTGCCTGAGCAGCATCTACGAGGACCTCAAGATGTACCAA
GTGGAGTTCAAGACCATGAACGCCAAGCTGCTGATGGATCCCAAGAGACAGATCTT
CCTTGATCAGAACATGCTGGCTGTGATCGACGAGCTGATGCAGGCACTGAACTTCAA
CAGCGAGACAGTGCCTCAGAAGTCTAGCCTGGAAGAACCCGACTTCTACAAGACCA
AGATCAAGCTGTGCATCCTGCTGCACGCCTTCCGTATCAGAGCCGTGACCATCGACA
GAGTGATGAGCTACCTGAACGCCTCCTGA
SEQ ID NO: 12, human single-chain IL-12 amino acid sequence
(with linkage to the pre-mature p35, with short signal
peptide removed from the fusion protein)
MCHQQLVISWFSLVFLASPLVAIWELKKDVYVVELDWYPDAPGEMVVLTCDTPEEDGI
TWTLDQSSEVLGSGKTLTIQVKEFGDAGQYTCHKGGEVLSHSLLLLHKKEDGIWSTDIL
KDQKEPKNKTFLRCEAKNYSGRFTCWWLTTISTDLTFSVKSSRGSSDPQGVTCGAATLS
AERVRGDNKEYEYSVECQEDSACPAAEESLPIEVMVDAVHKLKYENYTSSFFIRDIIKPD
PPKNLQLKPLKNSRQVEVSWEYPDTWSTPHSYFSLTFCVQVQGKSKREKKDRVFTDKT
SATVICRKNASISVRAQDRYYSSSWSEWASVPCSGGGGSGGGGSGGGGSRPVSLQCRLS
MCPARSLLLVATLVLLDHLSLARNLPVATPDPGMFPCLHHSQNLLRAVSNMLQKARQT
LEFYPCTSEEIDHEDITKDKTSTVEACLPLELTKNESCLNSRETSFITNGSCLASRKTSFM
MALCLSSIYEDLKMYQVEFKTMNAKLLMDPKRQIFLDQNMLAVIDELMQALNFNSETV
PQKSSLEEPDFYKTKIKLCILLHAFRIRAVTIDRVMSYLNAS
SEQ ID NO: 13, human 4-1BBL nucleotide sequence of NCBI
RefSeq NP_003802.1
ATGGAATACGCCAGCGACGCCTCTCTGGACCCTGAAGCTCCTTGGCCTCCAGCTCCT
AGAGCCAGGGCTTGTAGAGTGCTGCCTTGGGCTCTTGTGGCTGGACTTCTGCTTCTG
TTGCTCCTGGCTGCTGCCTGCGCAGTGTTTCTTGCTTGTCCATGGGCTGTGTCAGGAG
CCAGAGCATCTCCTGGATCTGCCGCTTCTCCCAGACTGAGAGAGGGACCTGAACTGA
GCCCTGATGATCCTGCTGGACTGCTCGACCTGAGACAGGGCATGTTTGCCCAGCTGG
TGGCCCAGAATGTGCTGCTGATTGATGGCCCTCTGAGCTGGTACAGCGATCCTGGAC
TTGCTGGCGTTAGCCTGACTGGAGGCCTGAGCTACAAGGAGGACACCAAAGAACTG
GTGGTGGCCAAGGCTGGCGTGTACTACGTGTTCTTTCAGCTGGAACTGCGGAGAGTG
GTGGCAGGCGAAGGATCTGGATCCGTGTCTCTGGCACTGCATCTGCAGCCTCTGAGA
TCTGCTGCTGGTGCAGCTGCCCTGGCTCTGACAGTTGATCTGCCTCCTGCCTCCAGCG
AAGCCAGAAACAGCGCCTTTGGCTTCCAAGGCAGACTGCTGCACCTGTCTGCTGGCC
AGAGACTGGGAGTGCACCTCCACACAGAAGCAAGAGCAAGACACGCCTGGCAGCTT
ACACAAGGCGCTACAGTGCTGGGCCTGTTCAGAGTGACACCTGAGATTCCAGCTGG
CTTGCCATCTCCTCGCAGCGAGTAATGA
SEQ ID NO: 14, human 4-1BBL amino acid sequence of NCBI
RefSeq NP_003802.1
MEYASDASLDPEAPWPPAPRARACRVLPWALVAGLLLLLLLAAACAVFLACPWAVSG
ARASPGSAASPRLREGPELSPDDPAGLLDLRQGMFAQLVAQNVLLIDGPLSWYSDPGLA
GVSLTGGLSYKEDTKELVVAKAGVYYVFFQLELRRVVAGEGSGSVSLALHLQPLRSAA
GAAALALTVDLPPASSEARNSAFGFQGRLLHLSAGQRLGVHLHTEARARHAWQLTQGA
TVLGLFRVTPEIPAGLPSPRSE

We claim:

1-6. (canceled)

7. A method for increasing an inflammatory response in a subject having a tumor, the method comprising intratumorally administering to the subject a recombinant modified Vaccinia virus Ankara (MVA) comprising a first nucleic acid encoding a tumor-associated antigen (TAA) and a second nucleic acid encoding IL-12, wherein the intratumoral administration of the recombinant MVA increases an inflammatory response in the tumor as compared to injection of MVA alone.

8. The method of claim 7, wherein the TAA is selected from the group consisting of carcinoembryonic antigen (CEA), mucin 1 cell surface associated (MUC-1), prostatic acid phosphatase (PAP), prostate specific antigen (PSA), human epidermal growth factor receptor 2 (HER-2), survivin, tyrosine related protein 1 (TRP1), tyrosine related protein 1 (TRP2), Brachyury, PRAME, FOLR1, HERV-K-env, HERV-K-gag, and combinations thereof.

9. The method of claim 7, wherein said recombinant MVA further comprises a third nucleic acid encoding 4-1BBL.

10. The method of claim 7, wherein the subject is human.

11-14. (canceled)

15. A method of inducing an increased inflammatory response in a peritoneal tumor of a subject, the method comprising intraperitoneally administering to the subject a recombinant modified Vaccinia Ankara (MVA) comprising a first nucleic acid encoding IL-12, wherein the intraperitoneal administration of the recombinant MVA generates an increased inflammatory response in the tumor as compared to an inflammatory response that would be generated by injection of MVA alone.

16. The method of claim 15, wherein said recombinant MVA further comprises a second nucleic acid encoding a tumor-associated antigen (TAA).

17. The method of claim 15, wherein said recombinant MVA further comprises a third nucleic acid encoding 4-1BBL.

18. A recombinant modified Vaccinia Ankara (MVA), comprising: (a) a first nucleic acid encoding IL-12; and (b) a second nucleic acid encoding a tumor-associated antigen (TAA); wherein the intratumoral administration of the recombinant MVA increases an inflammatory response in a tumor, reduces the growth rate and/or size of the tumor, and/or increases overall survival of the subject as compared to a non-intratumoral injection of said recombinant MVA or an injection of a recombinant MVA that does not comprise a nucleic acid encoding IL-12.

19. The recombinant MVA of claim 18, further comprising (c) a third nucleic acid encoding 4-1BBL.

20. The recombinant MVA of claim 18, wherein said TAA is an endogenous retroviral (ERV) protein.

21. The recombinant MVA of claim 18, wherein said TAA is selected from the group consisting of carcinoembryonic antigen (CEA), mucin 1 cell surface associated (MUC-1), prostatic acid phosphatase (PAP), prostate specific antigen (PSA), human epidermal growth factor receptor 2 (HER-2), survivin, tyrosine related protein 1 (TRP1), tyrosine related protein 1 (TRP2), Brachyury, FOLR1, PRAME, HERV-K-env, HERV-K-gag, p15, and combinations thereof.

22. A pharmaceutical combination comprising the recombinant MVA of claim 18 and a pharmaceutically acceptable carrier.

23. A pharmaceutical combination comprising the recombinant MVA of claim 18 and a second recombinant MVA comprising: (a) a first nucleic acid encoding a tumor-associated antigen (TAA); and (b) a second nucleic acid encoding 4-1BBL, wherein said TAA can be the same TAA as recited in claim 18 or can be a different TAA.

24. The pharmaceutical combination of claim 23 wherein said second recombinant MVA comprises a first nucleic acid encoding a TAA that is a different TAA than the one encoded by the recombinant MVA of claim 18.

25. The method of claim 7, wherein the subject has a plurality of tumors and said recombinant MVA is intratumorally administered to fewer than all of the tumors in said subject.

26. The method of claim 7, wherein the subject has at least one inaccessible tumor and at least one accessible tumor, the method further comprising intratumorally administering to at least one accessible tumor in the subject a recombinant MVA comprising at least one first nucleic acid encoding a TAA and a second nucleic acid encoding 4-1-BBL, whereby the growth of the inaccessible tumor is decreased or stopped.

27. The method of claim 15, whereby the growth of at least one tumor in the subject is decreased or stopped.

28. The method of claim 15, further comprising intraperitoneally administering to the subject a boosting dose of the same recombinant MVA.
